(19) United States
(12) Patent Application Publication
(10) Pub. No.: US 2008/0261258 A1
(43) Pub. Date: Oct. 23, 2008

(54) Immune Cell Biosensors and Methods of Using Same

(75) Inventors: Milton G. Smith, Rockledge, FL (US); Keith D. Crawford, Westwood, MA (US)

Correspondence Address: FOLEY & LARDNER LLP, 111 HUNTINGTON AVENUE, 26TH FLOOR, BOSTON, MA, 02199-7610, US

(73) Assignee: Amaox, Inc.

(21) Appl. No.: 11/631,574

(22) PCT Filed: Jul. 11, 2005
(86) PCT No.: PCT/US05/24491

§ 371 Date: Jan. 28, 2008

Related U.S. Application Data
(60) Provisional application No. 60/586,546, filed on Jul. 9, 2004.
(60) Provisional application No. 60/612,454, filed on Sep. 23, 2004.
(60) Provisional application No. 60/613,733, filed on Sep. 28, 2004.
(60) Provisional application No. 60/614,924, filed on Sep. 29, 2004.

(51) Int. Cl. C12Q 1/02; C12M 1/00

(52) U.S. Cl. 435/29; 435/287.1

(57) ABSTRACT

The present invention relates to immunological cells that are useful in detecting changes in physiological states, which provide for methods of diagnosing diseases or monitoring the course of patient therapy. Also provided are arrays of antigen presenting cell-specific markers for detecting changes in physiological states, and methods of detecting such changes.

### FIELD OF THE INVENTION

The present invention relates to immunological cells that are useful in detecting changes in physiological states, which provide for methods of diagnosing diseases or monitoring the course of patient therapy.

### BACKGROUND

In many diseases such as cancer, autoimmune diseases or cardiovascular disorders peptides of normal or abnormal cellular proteins are presented on the cell surface which can not be found on the cell surface of healthy individuals. Inadequate antigen presentation in humans results in the failure of human immune system to control and clear many pathogenic infections and malignant cell growth. Successful therapeutic vaccines and immunotherapies for chronic infection and cancer rely on the development of new approaches for efficient antigen presentation to induce a vigorous immune response which is capable of controlling and clearing the offensive antigens.

The ability of T cells to recognize an antigen is dependent on association of the antigen with either MHC Class I (MHC-I) or Class II (MHC-II) proteins. For example, cytotoxic T cells respond to an antigen in association with MHC-I proteins. Thus, a cytotoxic T cell that kills a virus-infected cell will not kill a cell infected with the same virus if the cell does not also express the appropriate MHC-I protein. Helper T cells recognize MHC-II proteins. Helper T cell activity depends in general on both the recognition of the antigen on antigen presenting cells and the presence on these cells of “self” MHC-II proteins. This requirement to recognize an antigen in association with a self-MHC protein is called MHC restriction. MHC-I proteins are found on the surface of virtually all nucleated cells. MHC-II proteins are found on the surface of certain cells including macrophages, B cells, and dendritic cells (DCs) of the spleen and Langerhans cells of the skin.

A crucial step in mounting an immune response in mammals, is the activation of CD4+ helper T-cells that recognize major histocompatibility complexes (MHC)-II restricted exogenous antigens. These antigens are captured and processed in the cellular endosomal pathway in antigen presenting cells, such as dendritic cells. In the endosome and lysosome, the antigen is processed into small antigenic peptides that are presented onto the MHC-II in the Golgi compartment to form an antigen-MHC-II complex. This complex is expressed on the cell surface, which expression induces the activation of CD4+ T cells.

Other crucial events in the induction of an effective immune response in an animal involve the activation of CD8+ T-cells and B cells. CD8+ cells are activated when the desired protein is routed through the cell in such a manner so as to be presented on the cell surface as processed proteins, which are complexed with MHC-I antigens. B cells can interact with the antigen via their surface immunoglobulins (IgM and IgD) without the need for MHC proteins. However, the activation of the CD4+ T-cells stimulates all arms of the immune system. Upon activation, CD4+ T-cells (helper T cells) produce interleukins. These interleukins help activate the other arms of the immune system. For example, helper T cells produce interleukin-4 (IL-4) and interleukin-5 (IL-5), which help B cells produce antibodies; interleukin-2 (IL-2), which activates CD4+ and CD8+ T-cells; and gamma interferon, which activates macrophages. Since helper T-cells that recognize MHC-II restricted antigens play a central role in the activation and clonal expansion of cytotoxic T-cells, macrophages, natural killer cells and B cells, the initial event of activating the helper T cells in response to an antigen is crucial for the induction of an effective immune response directed against that antigen.

Peptides and proteins expressed in diseased cells can be used as markers for the identification of such abnormal cells. Furthermore, the detection of antibodies in serum or other body fluids directed to these peptides or proteins can also be used as indicator of risk or as prognostic indicator. However, the concentrations of these disease related peptides are quite low, and isolating and identifying them is usually only efficacious when the disease predominates in the individual, which by that time, usually precludes effective treatment. There remains a need in the art for a rapid and sensitive assay for detection of a pathological state in a mammal.

### SUMMARY OF THE INVENTION

The present invention is based on the plasticity of antigen presenting cells, and the highly specific metabolic changes APC's, particularly DC's undergo after they encounter antigens. These changes can be quantitated and when compared to reference positive (antigen exposed) and negative (naïve) controls of APC's, provide information about the immune state and microenvironments of the mammal from which they are obtained.

In one aspect, the invention provides a diagnostic method having the steps of obtaining from a mammalian subject a sample of blood having a subpopulation of antigen presenting cells, substantially isolating the antigen presenting cells from the blood sample, deriving a genomic or proteomic mammalian subject signature for the isolated antigen presenting cells wherein the mammalian subject signature indicates the metabolic state of the antigen presenting cells in the subject, deriving one or more genomic or proteomic reference signatures of antigen presenting cells from a reference subject having a disease state, and comparing the mammalian subject signature to the reference signature, wherein congruity between the mammalian subject signature and the reference signature indicates the presence of the disease state in the mammal. A mammalian subject is preferably a human, but can also be a veterinary subject such as a dog, cat, horse, pig, sheep, goat, or other mammal. In one embodiment, the antigen presenting cells are dendritic cells. In another embodiment, the disease state is a cancer or cell proliferative disorder. In yet another embodiment, the disease state is a pathogenic infection. In still another embodiment, the pathogenic infection is a viral infection. In yet still another embodiment, the pathogenic infection is a bacterial infection. In yet still another embodiment, the disease state is caused by a bacterial toxin, such as from staphylococcus B enterotoxin or botulinum toxin.

In another aspect, the invention provides an array having a plurality of addresses, each address having affixed thereto a sample of nucleic acid corresponding to genes expressed by an antigen presenting cell. In one embodiment, the array further includes a plurality of secondary addresses, each secondary address having affixed thereto a sample of nucleic acid corresponding to genes expressed by an antigen presenting cell that has encountered an antigen. In another embodiment, the antigen presenting cell is a dendritic cell. In yet another embodiment, the antigen is a cancer antigen. In still another embodiment, the antigen is a viral antigen. In even another embodiment, the antigen is a bacterial antigen. In still another embodiment, the antigen is a fungal antigen.

In still another aspect, the invention provides a diagnostic method including the steps of obtaining a population of isolated antigen presenting cells, culturing the antigen presenting cells in the presence of a food-borne pathogen thereby producing reference cells, the reference cells having a proteomic or genomic reference signature specific for the food-borne pathogen, and obtaining the reference signature obtaining a sample of a food product, culturing naïve antigen presenting cells with the sample food product, obtaining a sample signature from the cocultured antigen presenting cells, and comparing the sample signature to the reference signature, wherein congruity between the sample signature and the reference signature indicates the presence of the food-borne pathogen in the food product. In one embodiment, the antigen presenting cell is a dendritic cell. In another embodiment, the food-borne pathogen is a bacterial pathogen. In still another embodiment, the food-borne pathogen is a viral pathogen. In still another embodiment, the food-borne pathogen is a prion pathogen. In a related aspect, the invention provides for obtaining the antigen presenting cells from a livestock mammal, and assaying for APC exposure to a food-borne pathogen in the livestock mammal, and the consequent gene and protein expression changes in the APC that follow from antigen contact. In one embodiment, the antigen presenting cell is a dendritic cell. In another embodiment, the food-borne pathogen is a bacterial pathogen. In still another embodiment, the food-borne pathogen is a viral pathogen. In still another embodiment, the food-borne pathogen is a prion pathogen, for example, the prion that causes Bovine Spongiform Encephalopathy (BSE).

In even yet another aspect, the invention provides a diagnostic method including the steps of obtaining from a patient being treated for a disorder, a sample of blood having a subpopulation of antigen presenting cells, substantially isolating the antigen presenting cells from the blood sample, deriving a genomic or proteomic patient signature for the isolated antigen presenting cells wherein the patient signature indicates the metabolic state of the antigen presenting cells in the subject, deriving one or more genomic or proteomic reference signatures of antigen presenting cells from a reference subject having the same disorder as the patient, and comparing the patient signature to the reference signature, wherein the congruity between the patient signature and the reference signature decreases during the treatment, thereby indicating the efficacy of the treatment in treating the disorder. In one embodiment, the disorder is a cell proliferative disease or a cancer, and the treatment is administration of an antineoplastic agent. In another embodiment, the disorder is a cell proliferative disease or a cancer, and the treatment provokes an immune response against the cell proliferative disease. In yet another embodiment, the disorder is an autoimmune disease, and the treatment reduces the autoimmune response. In still another embodiment, the disorder is a bacterial infection, and the treatment is administration of an antibacterial agent. In even another embodiment, the disorder is a viral infection, and the treatment is administration of an antiviral agent. In even still another embodiment, the disorder is a fungal infection and the treatment is administration of an antifungal agent. In also another embodiment, the disorder is a genetic disorder, and the treatment is gene replacement therapy.

In another aspect, the invention provides for a antigen presenting cell, wherein the cell has been cultured in the presence of an antigen, and wherein the antigen expresses a plurality of genes that are specifically upregulated in response to antigenic challenge. In one embodiment, the antigen presenting cell is a dendritic cell. In yet another embodiment, the antigen is a cancer antigen. In still another embodiment, the antigen is a viral antigen. In even another embodiment, the antigen is a bacterial antigen. In still another embodiment, the antigen is a fungal antigen. In even another embodiment, the antigen is a prion antigen. In one aspect, the specific polypeptides that are produced in response to antigen contact (marker proteins) are isolated. These are used to raise antibodies, which are used in subsequent assays involving isolated APC's from patients, whereby expressed polypeptides in the patient isolated APC's are identified, i.e., qualitatively and quantitatively by immunological assays, e.g., ELISA, FACs, RIA and similar techniques.

In another aspect, the invention provides for determining the proteomic signature of an antigen presenting cell that has been exposed to an antigen. In one embodiment, the proteomic signature is obtained by subjecting the antigen presenting cell to SELDI mass spectroscopy. In another embodiment, the proteomic signature is obtained by subjecting the antigen presenting cell to MALDI-O-TOF and other forms of mass spectroscopy. In yet another aspect the invention provides for proteomic signatures obtained from antigen presenting cells that have been exposed to an antigen. In one embodiment, the antigen presenting cell is a dendritic cell. In yet another embodiment, the antigen is a cancer antigen. In still another embodiment, the antigen is a viral antigen. In even another embodiment, the antigen is a bacterial antigen. In still another embodiment, the antigen is a fungal antigen. In even another embodiment, the antigen is a prion antigen.

In one aspect the invention includes a diagnostic method including the steps of: obtaining from a mammal having a disorder, a sample of blood having a subpopulation of antigen presenting cells; substantially isolating the antigen presenting cells from the blood sample; deriving one or more marker polypeptides from the antigen presenting cells, where the marker polypeptide is expressed in the antigen presenting cell in response to antigen contact and where the antigen contacted is associated with or the causative agent of the disorder; obtaining an antibody to the marker polypeptide; and detecting in the antigen presenting cells of a subject, the presence or absence of a polypeptide that binds to the antibody, wherein the presence of the polypeptide confirms the presence of the disorder in the subject. Detection of polypeptides that bind antibodies can be performed using assays such as an ELISA, RIA, FRET, FACs and other immunological detection methodologies. In one embodiment, the antigen presenting cells are dendritic cells. In one embodiment, the disorder is a cancer or cell proliferative disorder. In one embodiment, the disorder is a pathogenic infection. In one embodiment, the disorder is a viral infection. In one embodiment, the disorder is a bacterial infection. In one embodiment, the disorder is a prion infection. In one embodiment, the disorder is a fungal infection.

In another aspect, the invention includes a method of diagnosing exposure to an antigen comprising the steps of detecting the amount of protein/gene expression present in a sample of mammalian tissue or mammalian body fluids that has not been exposed to the antigen. Then the amount of protein/gene expression present in a sample of mammalian tissue or mammalian body fluids that has been exposed to the antigen is detected. A determination of the difference in the detected amount of protein/gene expression between the exposed and unexposed samples is made. A comparison of the difference to a library of expected protein/gene expression for predetermined antigens is made. Finally, an evaluation is made whether the difference indicates the exposure to a particular antigen. The present invention is particularly useful because it can provide a diagnosis of whether a person has been exposed to an antigen before the onslaught of any symptoms. The present invention is also directed to a method of diagnosing exposure to an antigen comprising the steps of detecting the patterns of gene expression/proteins present in a sample of mammalian tissue or mammalian body fluids from persons that have been potentially exposed to the antigen, determining the relative amounts of expression of a panel of genes or proteins relative to house keeping genes and proteins expressed in those tissues from the potentially exposed individuals, comparing the relative amount differences to a library of expected gene expression/proteins for predetermined antigens; and evaluating whether the differences indicate that exposure has occurred to a known, catalogued, toxic agent, to a previously unknown antigen, or to a antigen mixed with potentiating agents. Housekeeping genes are genes that tend not to change upon exposure to antigens.

In another aspect, the invention provides a method of diagnosing cancer in a mammalian subject comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to a cancer; wherein congruency between the subject signature and the reference signature indicates the subject has the cancer. Cancers amenable to detection are discussed below. Fluid samples that can be screened include: blood, plasma, bone marrow, pericardial, pleural, ascitic, and synovial fluids, cerebrospinal fluids, sputum, urine, and lymphatic fluids.

In yet another aspect, the invention provides, a method for identifying exposure of a mammalian subject to a pathogen or toxin comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to the pathogen or toxin; wherein congruency between the subject signature and the reference signature indicates exposure of the subject to the pathogen or toxin. The pathogen or toxin can be bacterial in origin, such as the organisms or toxins including: Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria resistant to penicillins, tetracyclines, spectinomycin, and fluoroquinolones; Methicillin-resistant Staphylococcus Aureus (MRSA); drug-resistant Streptococcus pneumoniae; fluoroquinolone and other drug resistant Salmonella serogroup Typhi; Vancomycin-Intermediate/Resistant Staphylococcus aureus; and Vancomycin-resistant Enterococci. The pathogen or toxin can also be Anthrax toxin; Arenavirus; Bacillus anthracis (anthrax); Clostridium botulinum toxin); Brucella species; Burkholderia mallei; Burkholderia pseudomallei (melioidosis); Chlamydia psittaci; Cholera toxin; Clostridium botulinum toxin (botulism); Clostridium perfringens; Ebola virus hemorrhagic fever; Nipah virus; hantavirus; Epsilon toxin of Clostridium perfringens; Escherichia coli including strain O157:H7; Shigella; Francisella tularensis; Glanders (Burkholderia mallei); Lassa fever; Marburg virus hemorrhagic fever; Melioidosis (Burkholderia pseudomallei); Psittacosis (Chlamydia psittaci); Q fever (Coxiella burnetii); Ricin toxin from Ricinus communis (castor beans); Rickettsia prowazekii; Salmonella Typhi and other Salmonella species; Shigella; Smallpox; Staphylococcal enterotoxin B; Typhus fever; Variola major; Vibrio cholerae (cholera); Viral encephalitis; alphaviruses such as Venezuelan equine encephalitis, eastern equine encephalitis, and western equine encephalitis; Filoviruses; Arenaviruses such as Lassa, and Machupo; Vibrio cholerae; Cryptosporidium parvum; and Yersinia pestis. Alternatively, the pathogen or toxin is prion, such as BSE.

In another aspect, the invention provides a method of detecting pathogen or toxin contamination in a sample comprising: obtaining a sample; incubating the sample for a period of time with a population of naïve antigen presenting cells thereby contacting the antigen presenting cells with the sample; isolating and purifying the sample contacted antigen presenting cells; obtaining a proteomic signature for the sample contacted antigen presenting cells; and comparing the proteomic signature from the sample contacted antigen presenting cells to at least one reference signature, the reference signature comprising a proteomic signature for reference antigen presenting cells that have been exposed to the pathogen or toxin; wherein congruency between the sample contacted signature and the reference signature indicates exposure of the subject to the pathogen or toxin. The sample may also be a food product.

The invention provides a method of diagnosing infection in a mammalian subject from a bacterial pathogen comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to a bacterial pathogen; wherein congruency between the subject signature and the reference signature indicates an active bacterial infection of the subject by the pathogen. Pathogens amenable to detection include: Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria resistant to penicillins, tetracyclines, spectinomycin, and fluoroquinolones; Methicillin-resistant Staphylococcus Aureus (MRSA); drug-resistant Streptococcus pneumoniae; fluoroquinolone and other drug resistant Salmonella serogroup Typhi; Vancomycin-Intermediate/Resistant Staphylococcus aureus; and Vancomycin-resistant Enterococci. In alternative embodiments, the invention provides for assaying the blood of the mammalian subject for the presence of biomarkers for sepsis. Biomarkers for sepsis include D-dimer; apolipoprotein A1; beta-2 microglobulin; C-reactive protein; epidermal growth factor; endothelin-1; eotaxin; Factor VII; fibroblast growth factor-9; basic fibroblast growth factor; fibrinogen; granulocyte chemotactic protein-2; granulocyte-macrophage colony stimulating factor; growth hormone; glutathione S-transferase; gamma interferon; IgA; IL-10; IL-11; IL-12p70; IL-17; IL-18; IL-1beta; IL-2; IL-3; IL-4; IL-5; IL-6; IL-7; insulin; gamma interferon inducible protein 10; KC; leptin; leukemia inhibitory factor; lymphotactin; monocyte chemoattractant protein-1/JE; monocyte chemoattractant protein-3; monocyte chemoattractant protein-5; macrophage colony stimulating factor; macrophage derived chemokine; macrophage inflammatory protein-1 alpha; macrophage inflammatory protein-1 beta; macrophage inflammatory protein-1 gamma; macrophage inflammatory protein-2; macrophage inflammatory protein-3 beta; myoglobin; oncostatin M; RANTES; stem cell factor; aspartate amino transferase; tissue inhibitor metalloproteinase-1; tumor necrosis factor-alpha; tissue factor; thrombopoietin; vascular cell adhesion molecule-1; vascular endothelial growth factor; and von Willebrand factor. Other biomarkers for sepsis are acute phase proteins.

These aspects and other features will be apparent from the discussion that follows.

### DETAILED DESCRIPTION

The present invention is based on the observed plasticity of antigen presenting cells (APC), and their use for the rapid detection of specific changes in gene and protein expression occurring in human dendritic cells and monocytes in response to exposure to pathogens, tumors, and hazardous agents. Antigen presenting cells, particularly dendritic cells (DC) macrophages and monocytes (M), neutrophils, T_{H1} and T_{H2} cells, NK cells and B-cells, are constantly sampling the various microenvironments found in the mammalian body. For example, DC cells are found in an immature state in most tissues (CD1a+, CD83^{Low}), where they recognize and phagocytose pathogens and other antigens (see, Poindexter, et al., (2004) Breast Cancer Res. 6(4):408-415). Platelets, although not cells per se but cell fragments of a megakaryocyte, can also bind and phagocytose infectious microorganisms and serum proteins, and can be considered as a reservoir for the detection of pathogens and cell fragments, such as tumor cells and apoptotic cell debris (see, Youssefian, et al., Host defense role of platelets: engulfment of HIV and Staphylococcus aureus occurs in a specific subcellular compartment and is enhanced by platelet activation. Blood. 2002 Jun. 1; 99(11):4021-9). Accordingly, an antigen presenting cell generally refers to those cells (and cell fragments) that internalize antigens, and possess the capacity to present these antigens to other cells. Exemplary antigen presenting cells include but are not limited to cells of lymphoid lineage such as T cells, B cells, lymphoid related dendritic cells and natural killer cells, and cells of the myeloid lineage such as myeloid related dendritic cells, macrophages, monocytes, megakaryocytes, platelets, granulocytes and neutrophils. Preferred are highly phagocytotc cells such as macrophages, monocytes and dendritic cells.

Direct contact with antigens or other pathological agents leads to the maturation of these antigen presenting cells, which is characterized by an increase in antigen presentation, expression of costimulatory molecules, expression of cytokines, and subsequent stimulation of naïve T cells in the lymphoid organs, as well as other cell specific markers such as surface CD83 expression in dendritic cells. This maturation process is regulated by numerous corresponding changes in gene expression in these cells, that can be qualitatively and quantitatively measured. The series of gene expression changes that occur are highly specific, and are in specific response to the particular antigen to which the APC is exposed. APC can differentiate between, for example, particular peptides, glycopeptides, glycolipids, and initiate responses that are similar but not identical, when exposed to various antigens. The particular changes in gene and protein expression of the APC in response to antigenic challenge represent very specific measurable biological signatures, which can be used to identify that an APC has experienced an antigen, as well as the nature of the antigen itself, e.g., its chemical composition and source. In certain embodiments, isolation of APC permit the subsequent extraction and isolation of phagocytosed antigens or even whole pathogens from the APC, which can be further characterized by MS or similar tools. For example, DC are known to internalize viral and bacterial pathogens without killing the pathogen (Sundquist et al., 2004, and Jantsch et al., 2003). Accordingly, one object of the present invention includes methods of harvesting parts and/or the entire pathogen or antigen, in addition to obtaining genomic/proteonomic signatures of the pathogen or antigen. Another object of the invention is isolation of the APC polypeptides that are upregulated in response to antigen contact. These polypeptides are highly specific markers for antigen contact, and their expression are indicative that the APC has encountered a particular antigen. By isolating these polypeptides, in whole or in part, antibodies can be raised, which are used in subsequent assays to determine antigen contact.

For example, see, U.S. Pat. No. 6,316,197 to Das, et al., Method of diagnosing of exposure to toxic agents by measuring distinct pattern in the levels of expression of specific genes. Exposure of immature DCs to LPS stimulation contributes to their terminal differentiation into CD70(+) DCs (see, Iwamoto S., et al., Lipopolysaccharide stimulation converts vigorously washed dendritic cells (DCs) to nonexhausted DCs expressing CD70 and evoking long-lasting type 1 T cell responses, J Leukoc Biol. 2005 Apr. 27; [Epub ahead of print]). See also, Kumar, A., Kurl, R. N., Kryworuchko, M., Diaz-Mitoma, F., & Sharma, S. 1995. Differential effect of heat shock on RNA metabolism in human Burkitt's lymphoma B-cell lines. Leuk. Res, 19(11): 831-840, which details an association between EBV transformation and enhanced expression of c-myc and poly-A polymerase (PAP) activity. See also, Hwang S L., et al., Indoleamine 2,3-dioxygenase (IDO) is essential for dendritic cell activation and chemotactic responsiveness to chemokines, Cell Res. 2005 March; 15(3):167-75, describing the upregulation of IDO in response to LPS or TNF-alpha stimulation; and Shi J., et al., Cancer Sci. 2005 February; 96(2): 127-33, which describes human cord blood monocyte-derived DC's acquiring the ability to kill hematological tumor cells, after activation with lipopolysaccharide (LPS) or gamma-interferon (IFN-gamma), associated with the enhanced TNF-alpha-related apoptosis-inducing ligand (TRAIL) expression in cord blood DC cytoplasm. See also, Smith, A. P., et al., J. Virol. 2005 March; 79(5):2807-13, which details that human herpesvirus-6 (HHV-6), but not the closely related betaherpesvirus HHV-7, dramatically suppressed the secretion of interleukin-12 (IL-12) p70 by DC, while the production of other cytokines that influence DC maturation, i.e., IL-10 and tumor necrosis factor alpha, was not significantly modified. Komer, et al., described the upregulation of macrophage 103 genes upregulated in response to Bacillus anthracis lethal toxin (LeTx). Similarly, Tucker et al., describes LeTx cleavage of mitogen activated protein kinase kinases (MAPKKs) in a variety of different APC cell types. Expression of genes regulated by MAPKK activity did not change significantly, yet a series of genes under glycogen synthase kinase-3-beta (GSK-3beta) regulation changed expression following LeTx treatment. See also, Green, S. J., Scheller, L. F., Marletta, M. A., Seguin, M. C., Klotz, F. W., Slayter, M., Nelson, B. J., & Nacy, C. A. 1994. Nitric oxide: cytokine-regulation of nitric oxide in host resistance to intracellular pathogens. Immunol. Lett, 43(1-2): 87-94, which describes regulation of nitric oxide (NO) production by APC in response to contact with Leishmania major, tularemia (Francisella tularensis), Mycobacterium bovis (BCG), and Plasmodium berghei. See also, Hernychova, L., Kovarova, H., Macela, A., Kroca, M., Krocova, Z., & Stulik, J. 1997. Early consequences of macrophage-Francisella tularensis interaction under the influence of different genetic background in mice. Immunol. Lett, 57(1-3): 75-81; and Clemens, D. L., Lee, B. Y., & Horwitz, M. A. 2004. Virulent and avirulent strains of Francisella tularensis prevent acidification and maturation of their phagosomes and escape into the cytoplasm in human macrophages. Infect. Immun., 72(6): 3204-3217. See also, Ng, L. C., Forslund, O., Koh, S., Kuoppa, K., & Sjostedt, A. 2003. The response of murine macrophages to infection with Yersinia pestis as revealed by DNA microarray analysis. Adv. Exp. Med Biol, 529: 155-160, which details a total of 22 different genes as up-regulated in response to the Y. pestis infection. These genes include unknown EST's, cytokines, enzyme of cytokine, receptors, ligands, transcriptional factors, inhibitor of transcriptional factor, proteins involved with the cytoskeleton, and 7 genes that encode for factors known to be associated with cell cycling and cell proliferation, with 3 of them playing a role in apoptosis. See also, Saban, M. R., Hellmich, H., Nguyen, N. B., Winston, J., Hammond, T. G., & Saban, R. 2001. Time course of LPS-induced gene expression in a mouse model of genitourinary inflammation. Physiol Genomics, 5(3): 147-160, which details that LPS treatment of APC downregulated the expression transcription factors, protooncogenes, apoptosis-related proteins (cysteine protease), intracellular kinases, and growth factors. Gene upregulation in response to LPS was observed in a cluster including the interleukin-6 (IL-6) receptor, alpha- and beta-nerve growth factor (alpha- and beta-NGF), vascular endothelial growth factor receptor-1 (VEGF R1), C—C chemokine receptor, and P-selectin. Another tight cluster of genes with marked expression included the protooncogenes c-Fos, Fos-B, Fra-2, Jun-B, Jun-D, and Egr-1. Almost all interleukin genes were upregulated as early as 1 h after stimulation with LPS. Nuclear factor-kappaB (NF-kappaB) pathway genes collected in a single cluster with a peak expression 4 h after LPS stimulation. In contrast, most of the interleukin receptors and chemokine receptors presented a late peak of expression 24 h after LPS exposure. See also, Mendis, C., Das, R., Hammamieh, R., Royaee, A., Yang, D., Peel, S., & Jett, M. 2005. Transcriptional response signature of human lymphoid cells to staphylococcal enterotoxin B. Genes Immun., 6(2): 84-94.

Thus these polypeptides, and other APC polypeptides provide for protein markers that are indicative of antigen contact. In one aspect, these polypeptide markers are isolated and used to raise antibodies. The anti-APC marker antibodies are then useful in assays that can be used to detect expression of APC marker polypeptides in cells obtained from patients suspected of antigen exposure. In one embodiment, the anti-APC marker antibodies are used in assays that employ immunological detection methods, such as fluorescent activated cell sorting (FACS), fluorescence resonance emission tomography (FRET), radioimmunoassay (RIA) and enzyme linked immunosorbant assays (ELISA). Other immunological detection assays are know to those of skill in the are and are suitable for the detection methods described herein.

Accordingly, changes in APC in response to antigenic challenge can be used to assay for persons in presymptomatic (not ill) state, and can be used to monitor the progression of a disease, or the efficacy of a therapeutic regimen in treating the disease. For example see Bernardo, K., Pakulat, N., Fleer, S., Schnaith, A., Utermohlen, O., Krut, O., Muller, S., & Kronke, M. 2004. Subinhibitory concentrations of linezolid reduce Staphylococcus aureus virulence factor expression. Antimicrob. Agents Chemother., 48(2): 546-444, which describes the influence of the antibiotic linezolid on the secretion of exotoxins by Staphylococcus aureus was analyzed by sodium dodecyl sulfate-polyacrylamide gel electrophoresis combined with matrix-assisted laser desorption ionization-time of flight mass spectrometry and Western blot analysis. Similarly, changes in APC in response to antigenic challenge can be used to assay for persons who have been exposed to biological agent(s)—and can be used in early diagnosis of the high risk exposed individual; as well as for monitoring persons who are in the early stages of developing symptoms. For example, changes in APC in response to antigenic challenge from a viral or bacterial pathogen can provide for rapid identification of these pathogens, and may predate the eventual pathogen appearance in plasma by many hours or days. In a related application, the invention can be used to monitor the effectiveness of a vaccination, by assaying for DC interaction with one or more components of the vaccine. Similarly, changes in APC in response to antigenic challenge from tumors permit the detection of tumors before an individual becomes symptomatic, thereby permitting early aggressive treatment. Also, changes in APC in response to exposure to industrial chemicals, or biowarfare agents may provide for identification of the unknown etiological agent to which an individual may be exposed.

APC's serve as the body's natural immune biosensor. These cell types circulate through all tissues of the body and are responsible for surveying most if not all tissues of the body by sampling the microenvironment. In doing so, they seek out areas of tissue that have a danger signal, i.e., increased mitotic activity, or viral/bacterial infections (Crawford et al., 2003). Once this signal is detected, APC's, initiate the early transcriptional changes, which lead to cell surface antigen expression and inflammatory mediator release (Crawford et al., 2003). These cellular modifications are required for recruitment of other inflammatory cells to the site of involvement and improved immune cell-to-cell contact. Antigen presenting cells such as DC's possess pattern recognition receptors, which allow them to bind to and discriminate between various pathogens (Chaussabel et al., 2003). Other receptors include Toll-like receptors, ICAM's such as ICAM-1, DCSIGN, and others.

As described above, APC's generate unique gene signatures in response to exposure to various pathogens. Studies of discordant gene expression in DC and macrophages infected with bacteria, Candida, influenza, or different parasites using oligonucleotide arrays have suggested that of the approximately 6800 genes samples, about 1300 genes demonstrate significant modulation in expression patterns after exposure to antigens (see, Huang et al, The plasticity of dendritic cell responses to pathogens and their components, Science, 294: 2001). For example, DC express C-type lectins as pathogen recognition receptors, for example, the DC-specific ICAM-3 grabbing nonintegrin (SIGN)/CD209, which has been identified as the HIV-1 receptor on DC, as well as for surface glycans for Mycobacterium tuberculosis, Helicobacter pylori, Leishmania mexicana, Schistosoma mansoni, and other pathogens (see, Appelmelk et al. (2003), J. Immunol., 170:(4):1635-9). See also Hofer et al., (2001) Immunol. Rev., June:181:5-19, and Pulendran et al., (2001), J. Immunol. November:167(9):5067-76. These receptors and the cellular pathways they interact with, provide unique markers for monitoring DC activation and response. More particularly, changes in DC response are measurable and provide pathogen specific signatures evidencing DC interaction with particular disease agents. See, Machein, U. & Conca, W. 1997. Expression of several matrix metalloproteinase genes in human monocytic cells. Adv. Exp. Med Biol, 421: 247-251, detailing several MMP genes are transcriptionally active in the cells tested after exposure to a variety of stimuli such as phorbol ester, lipopolysaccharide (LPS) and staphylococcal enterotoxin B (SEB).

Hazardous environmental agents are also detectable by the methods described herein, as they either can provoke an APC specific immune cell response themselves, or will destroy cells and tissues causing an increase in inflammation, extravasation, and activation of APC's in response to cytokines and various cellular factors. These properties of human APC's make them suitable for the rapid detection of exposure to any pathogenic substance, for example an infectious pathogen, tumor, toxin or toxic industrial chemical (TIC), or weapon of mass destruction (WMD).

The APC's described, preferably monocytes, and most preferably DC are useful to detect changes in the physiology of a subject, in response particularly to diseases, such as infectious diseases and cancers. As used herein, the term antigen is broadly used to refer to any composition that is generally foreign to a healthy mammal, or is native to the mammal but is mutated, aberrant or found in increased concentrations in the mammal having a pathological condition. An antigen thus includes whole pathogens such as bacteria, viruses, fungi, protozoa, as well as one or more components of a pathogen, for example a bacterial antigen includes lipopolysaccharide (LPS), teichoic acid, and peptidoglycan, a viral antigen includes a viral coat protein such as gp120 of HIV or hemagglutinin or neuraminidase of influenza, and a fungal antigen includes the cell-wall derived protein mannin. Prions are also antigenic, displaying specific peptide sequences associated with disease states (the normal cellular protein PrP^{C} and abnormal, disease-producing protein PrP^{Sc}). Antigens also include proteins and peptides associated with tumors, such as carcinoembryonic antigen (CEA) and aberrantly glycosylated mucin (MUC) as well as numerous other tumor specific antigens and proteins such as bcl-2, survivin, hepsin and the like. Accordingly, the common characteristic of an antigen, or antigenic agent, is the effect it has on an APC in that it causes specific biochemical changes in the APC such as the upregulation of antigen presentation proteins and co-receptors, as well as causing maturation and proliferation of APC's, tissue migration, and other properties that are indicative of exposure to an antigen. A detectable increase in APC is one where for example a two-fold or greater increase in the number of APC's are induced to develop or activate or mature in the mammal exposed to the antigen relative to those levels of APC's in the non-exposed or healthy mammal. Assay techniques for determining DC and other blood cells are well known in the art, for example but not limited to FACS using mature DC cell markers CD2+ and CD83+, or immature marker CD1a+.

In one aspect, the invention provides immune cell based methods for monitoring a patient's response to a disease state. Determining the pathogen or tumor-specific genomic and proteomic expression patters, or signatures, provides an improved method of on-going monitoring of the patient's immune response to the disease state. This information is used in conjunction with other relevant medical information such as decrease in tumor mass or tumor burden for a cancer patient, or a decrease in viral load for an HIV infected patient, or the clearance of mycobacteria in a tuberculosis patient, to allow monitoring of therapeutic efficacy, for example, in response to chemotherapy, anti-viral therapy, or administration of antibiotics.

APC's thus provide a useful diagnostic tool for identifying antigens and for monitoring the health of individuals, based upon changes in their cellular metabolism. Measurable changes occur in expression of numerous genes, proteins and secretory factors such as cytokines, and the antigens can also be detected in the cytoplasm of the APC (such as in the cytoplasm of platelets). As such, in one aspect the present invention provides for arrays of APC gene signatures, preferably monocyte or DC signatures. The array includes oligonucleotides, oligoribonucleotides or polypeptides of a plurality of APC marker genes and proteins, i.e., gene or protein products differentially expressed in antigen presenting cells. More preferably, the array includes from about 500-1000 specific markers at individual addresses in a matrix. Even more preferably, the array includes about 5,000, about 10,000, about 20,000 or greater genes or gene products, represented on the array. Most preferably, the array is a genome wide array, for example a mature DC cDNA array. Affymetrix and Illumina (both systems are complementary) arrays are exemplary. Individual genes or gene products may be duplicated on the array, for example as controls or for quantitative analysis of gene expression. The manufacture and use of such arrays are described in U.S. Pat. Nos. 6,741,344, 6,733,977, and 6,733,964. A method and apparatus for selectively applying a material onto a substrate for the synthesis of an array of, for example, oligonucleotides at selected regions or addresses on the substrate is further described by U.S. Pat. No. 6,667,394. The gene arrays produced are representative of the host reaction to the pathogen in great detail (typically 52,000 genes or more) and are not dependent the identification of one or a few genes (intrinsically biased), as is the case for identification by, e.g., Q-PCR. Proteomic data can be developed by a variety of techniques, for example but not limited to using surface plasmon resonance, or mass spectroscopy (MALDI or SELDI, etc). The combination of information obtained using genomic and proteomic approaches, in the format of a high throughput screen such as a DC gene array provides exceptionally specific diagnostic data, and thus a powerful tool for antigen identification or patient monitoring.

Numerous types of arrays are created, to develop APC based diagnostic arrays for a variety of purposes, but generally to obtain data sets for how APC's, particularly DC's, react upon exposure to different antigens. The use of a particular array depends on its chemical composition, and will vary depending on weather the array has nucleic acids, peptides, both, or other chemical moieties such as lectins etc. By way of general illustration, arrays such as Affymetrix's GeneChip® use biotin labeled cRNA prepared from cell extracts. About 5 micrograms total RNA are an appropriate starting material. The cRNA produced from the RNA sample is exposed to the array, allowed to hybridize to the appropriate target. The array is then washed and stained, e.g., with streptavidin phycoerythrin, then visualized using Affymetrix's GeneChip® Scanner 3000 or an Agilent GeneArray® Scanner. This technique as well as known immunological methods and other common methods of using proteomic and genomic arrays will be generally understood to those skilled in the art.

The arrays provide for the detection and identification of pathogens and pathogenic agents, as well as the detection and identification of transformed cells and tissues, using samples derived from subjects. Information about the disease state of a patient, that is, a patient data set, is obtained using one or more of the APC arrays described, by first obtaining a sample of blood from a subject, and then isolating the DC's from that blood sample. The DC signature from the patient is compared to one or more control DC signatures, for example, using the hybridization arrays described. The control DC signatures on the array minimally represent both the normal or healthy DC signatures and the abnormal or pathogenic DC signatures, for one or more disease states. Various other embodiments include additional control DC signatures that provide reference signatures for stages of various disease states, e.g., cancer stages.

The arrays and the data sets obtained there from are useful, for example, for discovering or diagnosing the existence of a genetic disease or chromosomal abnormality, or to provide information relating to identity, heredity or compatibility, diagnosing a predisposition to a disease or condition, diagnosing infection by a pathogenic organism, discovering or diagnosing neoplastic transformation of a cell or tissue, determining exposure to and identification of biowarfare or chemical warfare samples, or toxic industrial chemicals.

In one aspect, APC arrays are developed that are designed to identify the presence or absence of particular pathogens as well as their immunological consequences during the progression of the disease state they are associated with. For example, arrays are created that provide for the detection and monitoring of a viral infection such as HIV. The arrays include consensus APC signatures from immature or naïve APC's, from APC's obtained from an HIV exposed but asymptomatic person, APC's obtained from the exposed and early symptomatic person and from APC's in the later stage symptomatic person. Similar viral arrays are developed, for example ones useful for diagnosing and monitoring hepatitis, neoplastic viruses, or other chronic or pathogenic viral infections. Diagnostic arrays that can be used to monitor viral vectors used in gene therapy are also preferred, e.g., those directed to vaccinia or poxviruses, and more particularly, those specific to the transformed vector, which should produce a different DC signature than the wild type vector. In yet another aspect, the arrays include human APC, particularly DC and macrophage cells that are exposed to pathogens on CDC priority list. These types of arrays will facilitate rapid emergency diagnosis, etiologic studies, response and treatment of exposed or potentially exposed individuals. In one embodiment, the arrays include human APC, particularly T_{H1} and T_{H2} cells, B-cells, neutrophils, DC and macrophage cells that are exposed to pathogenic bacteria. Arrays specific to homeland defense or military uses are also provided herein, as DC arrays specific to biological warfare pathogens provide for rapid detection and response to terrorist or enemy bioweapons attacks. Such arrays include smallpox arrays, bacillus anthracis arrays, clostridium botulinum arrays and other WMD pathogens. For example, arrays of human APC, particularly DC and macrophage cells that are exposed to toxic agents facilitates emergency diagnosis, response and treatment of exposed or potentially exposed individuals. In another embodiment, the array and patient data set obtained there from facilitate forensic or toxicology studies of an exposed individual.

In another aspect, the arrays are obtained from human APC, particularly DC and macrophage cells in patients having different tumors, including different stages of tumor growth. APC arrays are designed to identify the presence or absence of particular tumor antigenic markers, and the immunological consequence of the tumor on the patient during the progression of the patient's cancer. This type of array facilitates rapid diagnosis, tumor identification, and appropriate treatment of afflicted individuals. The following cancer types each result in specific APC responses, and are amenable to detection using the techniques described: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; AIDS-Related Cancers; AIDS-Related Lymphoma; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Non-Hodgkin's During Pregnancy; Lymphoma, Primary Central Nervous System; Macroglobulinemia, Waldenström's; Male Breast Cancer; Malignant Mesothelioma, Adult; Malignant Mesothelioma, Childhood; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma and Thymic Carcinoma Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma, Childhood; Vulvar Cancer; Waldenström's Macroglobulinemia; and Wilms' Tumor.

Arrays are created that provide for the detection and monitoring of various cancers such as breast cancer, colon cancer, ovarian cancer, uterine cancer, prostate cancer, glioma, melanoma, small and large cell carcinoma, leukemia, and other neoplastic and precancerous disease states. Markers such as aberrantly glycosylated MUC-1, or expression of CEA or hepsin are examples of common tumor markers known to be associated with most of the above tumors. Comprehensive listings including tumor-specific markers are known in the medical literature. Exemplary arrays include consensus APC signatures from immature or naïve APC's, and from APC's obtained from persons having stage 0, 1, 2, 3 or 4 graded tumors. Histological profiles and other medical data may be used in connection with the APC arrays to provide additional information about the disease state. The plasticity and specificity of response of APC's to cancers allows very specific identification of the cancer type, and the staging of the disease. As such, they also permit a medical professional to monitor the course of a therapeutic regimen, by monitoring changes in APC signatures during, for example, a chemotherapy regimen. For example, a patient with pancreatic cancer is provided with gemcitabine, and before and during the course of gemcitabine therapy DC's are extracted to the patient and used with a pancreatic cancer DC array. The array indicates the patient had grade 3 pancreatic cancer at the outset of the treatment, and indicates that one moth of gemcitabine treatment has caused the cancer to revert to a grade 2 stage, thereby indicating continued gemcitabine therapy for the patient.

In even another embodiment, arrays of APC's from individuals having a genetic disorder are created. Representative genetic disorders include for example, a disease state resulting from the presence of a gene, the expression product of the gene being a bioactive molecule that causes or contributes to the disease state, or the absence of a gene where the expression product of the gene in a healthy individual is a bioactive molecule that ameliorates or prevents the disease state. An example of the former is cystic fibrosis, wherein the disease state is caused by mutations in the CFTR protein. An example of the latter is PKU, where the disease state is caused by the lack of an enzyme permitting the metabolism of phenylalanine. Examples of genetic disorders appropriate for screening with the present assays and methods include, for example multiple sclerosis, endocrine disorders, Alzheimer's Disease, Amyotrophic Lateral Sclerosis, Lupus, Angelman syndrome, Charcot-Marie-Tooth disease, Epilepsy, Essential tremor, Fragile X syndrome, Friedreich's Ataxia, Huntington disease, Niemann-Pick Disease, Parkinson's Disease, Prader-Willi syndrome, Rett syndrome, spinocerebellar atrophy, Williams syndrome, Ellis-van Creveld syndrome, Marfan Syndrome, Myotonic dystrophy, leukodystrophy, Atherosclerosis, Best disease, Gaucher disease, glucose galactose malabsorption, Gyrate atrophy, Juvenile onset diabetes, Obesity, Paroxysmal nocturnal hemoglobinuria, Phenylketonuria, Refsum disease, and Tangier disease. Such arrays are useful in detecting a genetic disorder in a patient, and monitoring the patient having the genetic disorder during therapy. Similarly, the present assays provide for monitoring the course of gene therapy treatments, by monitoring the immunological state of the patient so treated, particularly for the appearance of the healthy gene product or for adverse reactions to the gene therapy vector.

While the above discussion has focused on using APC's in diagnostics to determine biological changes in a sample, in another embodiment, direct analysis of the fluids of a subject, such as blood, sputum, urine, saliva, mucus, cerebrospinal fluid, lymphatic fluid and the like can be subjected to assay. These samples are analyzed using various medical techniques, e.g., spinal tap to look for infection in cerebrospinal fluid, or laboratory techniques such as proteomic tools e.g., mass spectroscopy, and are generally known and will also be described below. Thus, the assays of the present invention may involve the screening of APC's for changes in conjunction with direct analysis of the bodily fluids of a subject provides an even more sophisticated detection and monitoring method.

APC's thus provide a highly specific and rapid means for monitoring biological changes in an organism, based on specific genomic and proteomic signatures that are typified by the DC in a particular state. The above discussion has centered on using APC's in assays that employ such common techniques as hybridization or immunological reactivity. Other proteomics tools are appropriate in determining changes in APC states. One particularly preferred method of obtaining a DC proteomic signature involves obtaining the mass spectra of the APC sample.

During the last decade, mass spectrometry (MS) has become an important analytical tool in the analysis of biological macromolecules. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). To perform MS, the samples under study are subjected to Energy Desorption/Ionisation (EDI) from a surface by input of energy. Typically EDIs are thermal desorption/ionisation (TDI), plasma desorption/ionisation (PDI) and various kinds of irradiation desorption/ionisation (IDI) such as by fast atom bombardment (FAB), electron impact, etc. Where a laser is used to ionize the sample, the process is called laser desorption/ionisation (LDI), such as matrix assisted laser desorption/ionisation (MALDI). Desorption may be assisted by presenting the MS analyte together with various helper substances or functional groups on the ionization surface, preferably such as surface-enhanced laser desorption/ionisation (SELDI).

For molecules of low molecular weight, mass spectrometry has long been part of the routine physical-organic repertoire for analysis and characterization of organic molecules by the determination of the mass of the parent molecular ion. Introduction of the so-called “soft ionization” methods, namely MALDI and ElectroSpray Ionization (ESI), permitted intact ionization, detection and exact mass determination of large molecules, i.e. well exceeding 300 kDa in mass, such as peptides and proteins (see, Fenn, J. B., et al., (1989) Science 246, 64-71; Karas M. & Hillenkamp F. (1988) Anal. Chem. 60, 2299-3001). In addition, by arranging collisions of the ionized parent molecule with other particles (e.g., argon atoms), the ionized parent molecule is fragmented, forming secondary ions by collision induced dissociation (CID). The fragmentation pattern/pathway very often allows the derivation of more detailed information, for example structural information about the molecule.

MALDI-MS and ESI-MS have been used to analyze nucleic acids as well as proteins (see, Nordhoff E., et al., (1997) Mass Spectrom. Rev. 15: 67-138). However, since nucleic acids are very polar biomolecules, that are difficult to volatize, there has been an upper mass limit for clear and accurate resolution. ESI would seem to be superior to MALDI for the intact desorption of large nucleic acids even in the MDa mass range (Fuerstenau S. D. & Benner W. H. (1995). Rapid Commun. Mass Spectrom. 9, 1528-38; Chen R., Cheng X., Mitchell et al., (1995). Anal. Chem. 67, 1159-1163).

A few reports on the MALDI-MS of large DNA molecules with lasers emitting in the ultraviolet (UV) have been reported (Ross P. L. & P. Belgrader (1997) Anal. Chem. 69: 3966-3972; Tang K., et al., (1994) Rapid Commun. Mass Spectrum. 8: 727-730; Bai J., et al., (1995) Rapid Commun. Mass Spectrum. 9: 1172-1176; Liu Y-H-, et al., (1995) Anal. Chem. 67: 3482-3490 and Siegert C. W., et al., (1997) Anal. Biochem. 243, 55-65. However, based on these reports it is clear that analysis of nucleic acids exceeding 30 kDa in mass by UV-MALDI-MS gets increasingly difficult with a current upper mass limit of about 90 kDa (Ross P. L. & P. Belgrader (1997) Anal. Chem. 69: 3966-3972). The inferior quality of the DNA UV-MALDI-spectra has been attributed to a combination of ion fragmentation and multiple salt formation of the phosphate backbone. Since RNA is considerably more stable than DNA under UV-MALDI conditions, the accessible mass range for RNA is up to about 150 kDa (Kirpekar F., et al., (1994). Nucleic Acids Res. 22, 3866-3870).

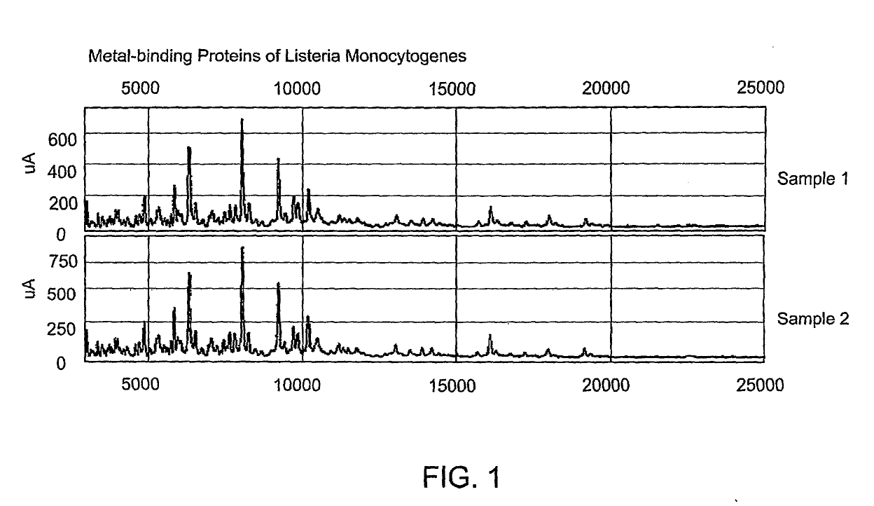

The analysis of nucleic acids by IR-MALDI with solid matrices (mostly succinic acid and, to a lesser extent, urea and nicotinic acid) has been described (Nordhoff, E. et al., (1992) Rapid Commun. Mass Spectrom. 6: 771-776; Nordhoff, E. et al., (1993) Nucleic Acids Res. 21: 3347-3357; and Nordhoff, E. et al., (1995) J. Mass Spec. 30: 99-112). The 1992 Nordhoff et al., paper reports that a 20-mer of DNA and an 80-mer of RNA were about the uppermost limit for resolution. The 1993 Nordhoff et al. paper, however, provides a distinct spectra for a 26-mer of DNA and a 104-mer of tRNA. The 1995 Nordhoff et al., paper shows a substantially better spectra for the analysis of a 40-mer by UV-MALDI with the solid matrix, 3-hydroxy picolinic acid, than by IR-MALDI with succinic acid (See FIGS. 1(d) and 1(e)). In fact the 1995 paper reports that IR-MALDI resulted in a substantial degree of prompt fragmentation.

In a Time-Of-Flight (TOF) mass spectrometer, the mass-to-charge ratio m/z of ions can be determined from their time of flight. Although it is always the mass-to-charge ratio m/z which is measured in mass spectrometry, with m being the mass and z being the number of elemental charges carried by the ion, in the following, for the sake of simplicity, only the mass m and its determination will be referred to. Since many types of ionization, such as MALDI, predominantly supply only single-charged ions (z=1), the difference ceases to exist in practice for these types of ionization. In a time-of-flight mass spectrometer (TOF-MS) which is equipped with an ion selector and a velocity-focusing reflector, it is possible to measure the daughter-ion or fragment-ion spectra of parent ions which are selected by the ion selector on the basis of their time of flight. The decay of parent ions into daughter or fragment ions can be induced by introducing excess energy during ionization (so-called PSD “Post Source Decay” spectra) or by applying other methods such as collisionally induced fragmentation. The parent ions and the daughter ions resulting from their decay enter the reflector simultaneously with the same average velocity but with different mass-proportional energies, such that they will be dispersed according to their mass within the reflector by their different energies.

Thus, mass spectroscopy, as well as other tools that permit detection of e.g., the infrared and ultraviolet absorption spectra, nuclear magnetic resonance spectra, as well as analytical profiles such as biomolecular interaction analysis (e.g., ELISA or surface plasmon resonance (SPR) profiles, see, Nedlekov et al., (2003) Appl. Env. Microbiol.) and other techniques to measure the physical properties of a sample, also provide methods for analyzing the samples. The information obtainable from methods using APC's described above, in connection with traditional laboratory methods, provides an integrated approach leading to the ability to resolve different properties of each sample under study. For example, where MS profiles for two samples display highly similar patterns, a second analysis such as an IR spectra, NMR spectra or SPR is used to provide additional comparative signatures and information. The result is an analytical signature profile, specific for each sample or sample under analysis, that provides for independent identification of the sample, (alone or in a mixture), and which can also provide, in certain embodiments, quantitative information about the sample such as concentration, as well as qualitative information, such as identification of other agents or materials in the sample mixture.

A preferred method utilizes mass spectroscopy to obtain proteomic signatures of APC's in healthy states and in response to challenge with antigens. Mass spectroscopy can also be used directly on mixtures suspected of containing the antigens or other contaminants. Most preferred analytical methods for obtaining these signatures includes SELDI, such as Ciphergen's ProteinChip® System Series 4000, or MALDI O-TOF, based on an orthogonal platform coupling the MALDI to the MS, such as Perkin Elmer's prOTOF™ 2000 MALDI O-TOF Mass Spectrometer.

In one aspect, proteomic signatures of APC, preferably DC, are obtained after challenge from toxins and organisms on the National Institute for Allergy and Infectious Diseases Biodefense Priority Pathogens List. The DC are cultured with the antigens or fragments thereof, as is described below. Proteomic signatures are obtained. These relevant antigens include Bacillus anthracis (anthrax), Clostridium botulinum, Yersinia pestis, Variola major (smallpox) and other pox viruses, Francisella tularensis (tularemia), and those causing Viral hemorrhagic fevers, Arenaviruses, such as LCM, Junin virus, Machupo virus, Guanarito virus, and those causing Lassa Fever, Bunyaviruses and Hantaviruses such as those causing Rift Valley Fever, Caliciviruses, Hepatitis A, B and C), viral encephalitides such as West Nile Virus. LaCrosse, California encephalitis, VEE, EEE, WEE, Japanese Encephalitis Virus, Kyasanur Forest Virus, Tickborne hemorrhagic fever viruses, Crimean-Congo Hemorrhagic fever virus, Tickborne encephalitis viruses, Yellow fever, Multi-drug resistant TB, Influenza, Other Rickettsias and Rabies, Flaviruses, Dengue, Filoviruses, Ebola, Marburg Burkholderia pseudomallei, Coxiella burnetii (Q fever), Brucella species (brucellosis), Burkholderia mallei (glanders), Ricin toxin (from Ricinus communis), Epsilon toxin of Clostridium perfringens, Staphylococcus enterotoxin B, Typhus fever (Rickettsia prowazekii), Food and Waterborne Pathogens, Bacteria such as Diarrheagenic E. coli, Pathogenic Vibrios, Shigella species, Salmonella, Listeria monocytogenes, Campylobacter jejuni, Yersinia enterocolitica), and Protozoa such as Cryptosporidium parvum, Cyclospora cayatanensis, Giardia lamblia, Entamoeba histolytica, Toxoplasma, Microsporidia. These biosamples are amenable to detection and identification, based on such criteria as lipoprotein content, glycoprotein content, membrane composition, the presence and absence of viral envelopes, expression of particular proteins such as virulence factors, and other biochemical profiles. See, for example, Dell A, Morris H R, Glycoprotein structure determination by mass spectrometry. Science. 2001; 291(5512):2351-6. See also, Rudd P. M., et al., Glycosylation differences between the normal and pathogenic prion protein isoforms. Proc Natl Acad Sci USA. 1999, 96(23):13044-9. See also, Beerman et al., The lipid component of lipoproteins from B. burdorferi: structural analysis, antigenicity, and presentation by human dendritic cells, Biochem. Biophys. Res. Comm., 267: 897-905 (2000). Analytical signatures are obtained from samples of cells, fluids and tissues of a subject exposed to (or suspected of exposure to) one or more toxins and organisms on the National Institute for Allergy and Infectious Diseases Biodefense Priority Pathogens List. The signatures of DC and fluids or tissues are compared to reference signatures to confirm exposure and to aid in monitoring treatment. For example, a blood sample is obtained from a subject suspected of having been exposed to smallpox. The sample is split into two aliquots; the DC recovered from one, and the plasma purified from the other. Both samples are subjected to mass spectroscopy. The DC signature is compared to reference signatures that provide positive and negative controls for exposed and naïve DC, and the plasma is assayed for the presence of variola virus. The signatures can confirm infection, before the patient becomes viremic or symptomatic, thus facilitating their quarantine.

The SELDI or MALDI O-TOF mass spectrometer signatures may profile the nucleic acids, proteins, carbohydrates and lipids of a microbial sample, but can preferably profile and obtain a signature for the whole pathogen. The signatures distinguish between microbial species, and varieties within the species, e.g., E. coli O157, stages of microbial growth, e.g., sporulative, vegetative, or in active growth, and relative age, as well as other characteristics such as pathogenicity, for example the pyrogenic exotoxin A production in group A streptococci, the cholera toxin in Vibrio cholerae, Shiga toxin-producing Escherichia coli (STEC), or enterotoxin production in enterohemorrhagic (EHEC) strains of E. coli. For example, Lai et al., discuss that sixty seven strains of Camobacterium, atypical Lactobacillus, Enterococcus durans, Lactobacillus maltaromicus and Vagacoccus salmoninarum were examined by Fourier transform infrared (FT-IR) spectroscopy. The effects of culture age and reproducibility over a six month period were also investigated. The results were analyzed by multivariate statistics and compared with those from a previous numerical phenetic study, a pyrolysis mass spectrometry (PyMS) study and with investigations which used DNA-DNA and 16S rRNA sequencing homologies. Taxonomic correlations were observed between the FT-IR data and these studies. Culture age was observed to have little effect on the spectra obtained. The reproducibility study indicated that there was correlation between spectra produced on two occasions over the six month period. It was concluded by Lai et al., that FTIR is a reliable method for investigating Carnobacterial classification, and may have further potential as a rapid method for use in Carnobacterium identification. See, Lai, S., R. Goodacre, et al. (2004). “Whole-organism fingerprinting of the genus Carnobacterium using Fourier transform infrared spectroscopy (FT-IR).” Syst Appl Microbiol 27(2): 186-91. Similarly, Lee et al discuss a bacterial analysis method coupling the flow field-flow fractionation (flow FFF) separation technique with detection by matrix-assisted laser desorption/ionization time-of-flight mass spectrometry. The composition of carrier liquid used for flow FFF was selected based on retention of bacterial cells and compatibility with the MALDI process. The coupling of flow FFF and MALDI-TOF MS was demonstrated for P. putida and E. coli. Fractions of the whole cells were collected after separation by FFF and further analyzed by MALDI-MS. Each fraction, collected over different time intervals, corresponded to different sizes and different growth stages of bacteria. See, Lee, H., S. K. Williams, et al. (2003). “Analysis of whole bacterial cells by flow field-flow fractionation and matrix-assisted laser desorption/ionization time-of-flight mass spectrometry.” Anal Chem 75(11): 2746-52. Likewise, Lefmann et al., discuss MALDI-TOF MS after base-specific cleavage of PCR amplified and in vitro-transcribed 16S rRNA gene (rDNA), used for the identification of mycobacteria. Full-length 16S rDNA reference sequences of 12 type strains of Mycobacterium spp. frequently isolated from clinical specimens were determined by PCR, cloning, and sequencing. For MALDI-TOF MS-based comparative sequence analysis, mycobacterial 16S rDNA signature sequences (approximately 500 bp) of the 12 type strains and 24 clinical isolates were PCR amplified using RNA promoter-tagged forward primers. T7 RNA polymerase-mediated transcription of forward strands in the presence of 5-methyl ribo-CTP maximized mass differences of fragments generated by base-specific cleavage. In vitro transcripts were subsequently treated with RNase T1, resulting in G-specific cleavage. Sample analysis by MALDI-TOF MS showed a specific mass signal pattern for each of the 12 type strains, allowing unambiguous identification. All 24 clinical isolates were identified unequivocally by comparing their detected mass signal pattern to the reference sequence-derived in silico pattern of the type strains and to the in silico mass patterns of published 16S rDNA sequences. A 16S rDNA microheterogeneity of the Mycobacterium xenopi type strain (DSM 43995) was detected by MALDI-TOF MS and later confirmed by Sanger dideoxy sequencing. Lefmann et al., concluded that analysis of 16S rDNA amplicons by MS after base-specific cleavage of RNA transcripts allowed fast and reliable identification of the Mycobacterium tuberculosis complex and ubiquitous mycobacteria (mycobacteria other than tuberculosis). See, Lefmann, M., et al., Novel mass spectrometry-based tool for genotypic identification of mycobacteria. J Clin Microbiol 42(1): 339-46 (2004). Thus, one object of the present invention include obtaining proteomic, genomic, lipid, carbohydrate, and whole organism signatures for bacterial pathogens. Preferably these are obtained using SELDI or MALDI O-TOF mass spectrometry, alone or in conjunction with other assays. Microbial identification is not limited to bacteria, and the analytic signatures of other pathogenic organisms thus include those of fungi, viruses, prions, and other infectious agents and pathogens.

The proteomic signatures derived from APC and those obtained by direct assessment of the pathogens from the fluids of a patient are used in the diagnosis of disease as described, but are particularly useful for monitoring the course of therapy, e.g., in response to antimicrobial compounds such as terbinafine, fluconizole, lamivudine, ciprofloxacin, vancomycin, penicillin, methicillin and other antibiotics. Signatures of tissues, fluids and cells of a subject therapeutically treated with antimicrobial compounds can also be analyzed for toxicity during such therapy. Detection of viral samples is described in, for example, Hong et al., which assayed for mutations in hepatitis B virus (HBV) permitting lamivudine resistance, that arise during prolonged treatment with that drug. Therapy with lamivudine frequently causes selection for HBV virions having amino acid substitutions in the YMDD motif of HBV DNA polymerase. MALDI-TOF MS genotyping detects HBV variants in a sensitive and specific manner. The assay in Hong et al., is based on PCR amplification and mass measurement of oligonucleotides containing sites of mutation of the YMDD motif. The MALDI-TOF MS-based genotyping assay described therein is sufficiently sensitive to detect as few as 100 copies of HBV genome per milliliter of serum, with superior specificity for determining mixtures of wild-type and variant viruses. When sera from 40 patients were analyzed, the MALDI-TOF MS-based assay correctly identified known viral variants and additional viral quasi-species not detected by previous methods, as well as their relative abundance. Hong et al., concluded the sensitivity, accuracy and amenability to high-throughput analysis makes the MALDI-TOF MS-based assay suitable for mass screening of HBV infected patients receiving lamivudine, and can help provide further understanding of disease progression and response to therapy. See, Hong et al., Detection of hepatitis B virus YMDD variants using mass spectrometric analysis of oligonucleotide fragments, J Hepatol. (2004). Thus, one object of the present invention includes the proteomic, genomic, lipid, carbohydrate, and whole organism signatures for viral pathogens, and analytic signatures of DC and other tissues, fluids and cells of a subject having a viral infection. Preferably these are obtained using SELDI or MALDI O-TOF mass spectrometry, alone or in conjunction with other assays.

Bonetto et al., discusses the elucidation of the structure and biological properties of the prion protein scrapie (PrP(Sc)) as fundamental to an understanding of the mechanism of conformational transition of cellular (PrP(C)) into disease-specific isoforms and the pathogenesis of prion diseases. They observed that a construct of 106 amino acids (termed PrP106 or miniprion), derived from mouse PrP was highly toxic to primary neuronal cultures, and induced a remarkable increase in membrane microviscosity. See, Bonetto V., et al., Synthetic miniprion PrP106, J Biol Chem. 277(35):31327-34 (2002). Accordingly, in still another aspect, the invention includes signatures of prion samples, and signatures of APC, and tissues, fluids and cells of a subject having a prion infection. Preferably these are obtained using SELDI or MALDI O-TOF mass spectrometry, alone or in conjunction with other assays.

It is yet another object of the invention to obtain the signatures, e.g., SELDI or MALDI O-TOF MS and other analytic signatures, from healthy and from diseased subjects, i.e., APC, fluids and tissues, for example in diseases characterized by various stages of physical degeneration, such as, cardiac muscle, kidney, or neural tissues, in various stages of infection, such as viral or bacterial, or in various stages of transformation, malignancy or tumorogenicity. In particular, cancers and premalignant tissues all undergo significant biochemical changes relative to nondiseased cells and tissues, that can be readily detected by spectral and other types of analytical methods. One example of this is the change in glycosylation patterns seen in the tumor associated antigen MUC-1 in many different cancers types, or the differential expression of chorioembrionic antigen (CEA), or tumor suppressor genes such as retinoblastoma (RB), p53, and cyclin dependent kinases cdk's. Numerous markers for cellular transformation and cancer are known in the medical literature, and all of these can be disease signatures for the purpose of the present invention. Likewise, these tissues exhibit changes to their metabolic states in response to treatment with chemotherapeutic samples and radiation. These changes are molecular signatures of a response to treatment, and are thus useful for the purposes described herein. Thus, the present methods can be used, for example, to identify and stage a particular tumor type, and monitor changes to the tumor over the course of therapy, such as chemotherapy or radiation. The methods described may also be used to monitor changes to healthy organs and tissues during such chemotherapy or radiation regimens, for example, to assess the systemic toxicity of the therapy for making adjustments to the course of treatment. In one embodiment, a toxicology profile for a chemotherapy regimen is provided. This profile comprises tissue specific molecular analytical signatures of a plurality mammalian organs and tissues in an untreated state, i.e., without exposure to a chemotherapy drug, as well as in response to a plurality of dosages of the drug. The profile can include a time dimension, i.e., dose response signatures of the tissues over a period of time. The present invention thus includes analytical signatures useful in the detection and treatment of disease. For example, Chaurand et al., determined that analysis of thin tissue sections of organs results in over 500 individual protein signals in the mass range of 2 to 70 kDa that directly correlate with the protein composition within a specific region of the tissue sample. Such profiling, including imaging MS, has been applied to multiple diseased tissues, including human gliomas and non-small cell lung cancer. Interrogation of the resulting complex MS data sets has resulted in identification of both disease-state and patient-prognosis specific protein patterns. See, Chaurand P, et al., Assessing protein patterns in disease using imaging mass spectrometry. J Proteome Res. 2004 March-April; 3(2):245-52. Likewise Ahmed et al., discuss differentially expressed proteins in the serum of ovarian cancer patients that may be useful as biomarkers of this disease. In Ahmed, a total of 24 serum proteins were differentially expressed in grade 1, 31 in grade 2, and 25 in grade 3 ovarian cancer patients. Six of the protein spots that were significantly upregulated in all groups of ovarian cancer patients were identified by nano-electrospray quadrupole time-of-flight mass spectrometry (n-ESIQ(q)TOFMS) and matrix-assisted laser desorption ionization time-of-flight mass spectrometry as isoforms of haptoglobin-1 precursor (HAP1), a liver glycoprotein present in human serum. Further identification of the spots at different pathological grades was confirmed by Western blotting and immunohistochemical localization using monoclonal antibodies against a haptoglobin epitope contained within HAP1. See, Ahmed N, et al., Proteomic-based identification of haptoglobin-1 precursor as a novel circulating biomarker of ovarian cancer. Br. J. Cancer 2004. Similarly, Bharti et al., discuss detection of serum tumor biomarkers at an earlier stage in order to improve the overall survival of cancer patients. Utilizing MALDI-TOF-Mass Spectrometry (MS) based protein identification techniques, a SCLC specific overexpressed protein was identified to be haptoglobin alpha-subunit, with its serum level correlating with the disease stage. The mean level of alpha-haptoglobin was increased in SCLC serum as compared to the normal controls. Serum HGF was also studied as potential tumor biomarker and was found to correlate with the disease status. See, Bharti A, et al., Haptoglobin alpha-subunit and hepatocyte growth factor can potentially serve as serum tumor biomarkers in small cell lung cancer. Anticancer Res. 2004 March-April; 24(2C): 1031-8. Several other tumor types are amenable to detection using the present methods. Iwadate et al., discuss the detection and response to chemotherapeutic treatment of gliomas. The biological features of gliomas, which are characterized by highly heterogeneous biological aggressiveness even in the same histological category, are precisely described by global gene expression data at the protein level. Iwadate et al., investigated whether proteome analysis based on two-dimensional gel electrophoresis and matrix-assisted laser desorption/ionization time-of-flight mass spectrometry could identify differences in protein expression between high- and low-grade glioma tissues. Proteome profiling patterns were compared in 85 tissue samples: 52 glioblastoma multiform, 13 anaplastic astrocytomas, 10 astrocytomas, and 10 normal brain tissues. Iwadate et al., could completely distinguish the normal brain tissues from glioma tissues by cluster analysis based on the proteome profiling patterns. Proteome-based clustering significantly correlated with the patient survival, and they could identify a biologically distinct subset of astrocytomas with aggressive nature. Iwadate et al., found that discriminant analysis extracted a set of 37 proteins differentially expressed based on histological grading. Among them, many of the proteins that were increased in high-grade gliomas were categorized as signal transduction proteins, including small G-proteins. Immunohistochemical analysis confirmed the expression of identified proteins in glioma tissues. See, Iwadate Y, et al., Molecular classification and survival prediction in human gliomas based on proteome analysis. Cancer Res. 2004 Apr. 1; 64(7):2496-501. Friedman et al., discuss two-dimensional difference gel electrophoresis (2-D DIGE) coupled with mass spectrometry (MS), used to investigate tumor-specific changes in the proteome of human colorectal cancers and adjacent normal mucosa. Friedman et al., investigated over 1500 protein spot-features in each paired normal/tumor comparison, and using DIGE technology with the mixed-sample internal standard, and made statistically significant quantitative comparisons of each protein abundance change across multiple samples simultaneously. Matrix-assisted laser desorption/ionization-time of flight and tandem (TOF/TOF) MS provided sensitive and accurate mass spectral data for database interrogation, resulting in the identification of 52 unique proteins (including redundancies due to proteolysis and post-translationally modified isoforms) that were changing in abundance across the cohort. See, Friedman D B, et al., Proteome analysis of human colon cancer by two-dimensional difference gel electrophoresis and mass spectrometry. Proteomics. 2004 March; 4(3):793-811. Hamler et al., discusses a two-dimensional liquid-phase separation scheme coupled with mass spectrometry (MS) for proteomic analysis of cell lysates from normal and malignant breast epithelial cell lines. Liquid-phase separations consist of isoelectric focusing as the first dimension and nonporous silica reverse-phase high-performance liquid chromatography (NPS-RP-HPLC) as the second dimension. Protein quantitation and mass measurement are performed using electrospray ionization-time of flight MS (ESI-TOF MS). Proteins are identified by peptide mass fingerprinting using matrix-assisted laser desorption ionization-time of flight MS and MALDI-quadrupole time of flight (QTOF)-tandem mass spectrometry (MS/MS). Hamler et al created mass maps that allowed visualization of protein quantitation differences between normal and malignant breast epithelial cells. Of the approximately 110 unique proteins observed from mass mapping experiments over the limited pH range, 40 (36%) were positively identified by peptide mass fingerprinting and assigned to bands in the mass maps. Of these 40 proteins, 22 were more highly expressed in one or more of the malignant cell lines. These proteins represent potential breast cancer biomarkers that could aid in diagnosis, therapy, or drug development. See, Hamler R L, et al., A two-dimensional liquid-phase separation method coupled with mass spectrometry for proteomic studies of breast cancer and biomarker identification. Proteomics. 2004 March; 4(3):562-77. Veenstra et al., discusses serum protein fingerprinting. Many proteomic studies have focused on the identification and subsequent comparative analysis of the thousands of proteins that populate complex biological systems such as serum and tissues. See, Veenstra T. D. et al., Serum protein fingerprinting. Curr Opin Mol Ther. 2003 December; 5(6):584-93. Accordingly, these proteomic, carbohydrate, nucleic acid, and lipid spectroscopic profiles or patterns provide for signatures of numerous tissues in both healthy and disease states, and from a diagnostic perspective indicate the presence of disease and can be used to monitor changes in the organism having the disease. In one embodiment, serum or lymphatic samples are used for obtaining such signatures. In another embodiment, DC are used. In other embodiments, APC provide the analytical signatures. In yet other embodiments, blood cells, muscle tissues, nervous tissues, epithelial tissues and connective tissues are assessed.

Another object of the present invention includes determining the chemical signatures of toxic industrial chemicals, and the consequential proteomic, genomic, lipid, and carbohydrate signatures of APC, tissues, fluids and cells of a subject that has been, or is suspected of being exposed to toxic industrial chemicals (TIC). Preferably these are obtained using SELDI or MALDI O-TOF mass spectrometry, alone or in conjunction with other analytical methods. The resultant signatures are stored in a database, and made available for diagnostic and therapeutic applications. A toxic industrial chemical is generally understood as a material that has a toxicity (LC50 by inhalation) of less than 100,000 Mg per min/M3 and an appreciable (undefined) vapor pressure at 20° C. The term TIC as used herein, includes Toxic Industrial Materials (TIM), generally regarded as any substance that in a given quantity produces a toxic effect in exposed personnel through inhalation, ingestion, or absorption. Examples of TICs and TIMs include fuels, oil, pesticides and herbicides, acids and bases, radiation sources, fertilizers, arsenic, chlorine, bromine, carbon disulfide, cyanide, metals (e.g., cobalt, lead, mercury, cadmium and thallium), phosgene and other organic and heavy metal toxins. Many TICs and TIMs are known in industry, and the above referenced agents are not intended to be comprehensive or limiting.

In another aspect, the invention provide for signatures, e.g., chemical, proteomic, genomic, lipid, carbohydrate, and whole organism signatures of agents of significance to national defense, such as biowarfare and chemical warfare agents (also known as WMD), and the proteomic, genomic, lipid, and carbohydrate signatures of APC, tissues, fluids and cells of a subject that has been, or is suspected of being exposed to such agents. Preferably these signatures are obtained with SELDI, MALDI O-TOF MS and other analytic methods. In particular, since spectral analysis provides a rapid and accurate detection means, it is possible to employ the present invention as part of a rapid or first response program, for field identification of biowarfare and chemical warfare agents in the samples.

The first step in the analytical process includes obtaining a sample of the agent (TIC, WMD) bacteria, virus, prion, cell, APC, fluid or tissue under study. The sample may be processed prior to examination, i.e., dissolved in water or a solvent, or used intact. Simple analytical methods may be used to gain rudimentary information about the sample. Collection of a mass spectrum and analysis thereof follows. The sample is applied to an inlet port on the MS, and if a mixture or a whole cell (or organism) may further contain one or more analytes, which may comprise lipid, carbohydrate, nucleic acid and/or peptide structure or any other inorganic or organic structure. Samples may undergo treatments prior to MS, where the sample may be transformed to one in which, the MS-analyte is a derivative of the starting analyte, the amount(s) of non-analyte species have been changed compared to the starting sample, the relative occurrence of different MS-analytes in a sample is changed compared to the starting sample, the concentration of an MS-analyte is changed relative the corresponding starting analyte in the starting sample, or sample constituents, such as solvents, have been changed and/or the analyte has been changed from a dissolved form to a solid form, for instance in a co-crystallized form. Such treatments include, for example, digestion into fragments of various sizes and/or chemical derivatization of an analyte. Digestion may be purely chemical or enzymatic. Derivatization includes so-called mass tagging of either the starting analyte or of a fragment or other derivative formed during a sample treatment protocol. Other treatments include purifying and/or concentrating the sample prior to analysis. Such treatments apply, for example, to analytes that are biopolymers comprising carbohydrate, lipid, nucleic acid and/or peptide structure. Alternatively, the sample may also pass through the microchannel structure without being changed.

### Sepsis and Infection

Sepsis is a severe illness caused by overwhelming infection of the bloodstream by toxin-producing bacteria. Sepsis is often life-threatening, especially in people with a weakened immune system or other medical illnesses. Sepsis is caused by a bacterial infection that can originate anywhere in the body. In hospitalized patients, common sites of infection include intravenous lines, surgical wounds, surgical drains, and sites of skin breakdown known as decubitus ulcers or bedsores, and infections of the kidneys (upper urinary tract infection), the liver or the gall bladder, the bowel (usually seen with peritonitis), the skin (cellulitis), and the lungs (bacterial pneumonia), and other sites. Numerous other pathological disease states may include sepsis as a component, for example bacterial meningitis may also be accompanied by sepsis. In children, sepsis may accompany infection of the bone (osteomyelitis). Complications of sepsis include septic shock, an impaired blood flow to vital organs (brain, heart, kidneys), and disseminated intravascular coagulation.

A change in mental status and hyperventilation may be the earliest signs of impending sepsis. A bacterial infection of the blood is often confirmed by a positive blood culture, though blood cultures may be negative in individuals who have been receiving antibiotics. In sepsis, blood pressure drops, resulting in shock. Major organs and systems, including the kidneys, liver, lungs, and central nervous system, stop functioning normally. An alternative name for sepsis is Systemic Inflammatory Response Syndrome (SIRS), which illustrates the immune effects of a systemic infection. Many different cytokines discussed below, play a role in the pathology of sepsis, and are biomarkers for bacterial infections and sepsis. Other symptoms include fever or hypothermia (low body temperature), hyperventilation, chills, shaking, warm skin, skin rash, rapid heart beat, confusion or delirium, and decreased urine output.

Clinical diagnosis of sepsis is suggested when the white blood cell count of the patient is either low or high, the platelet count is low, a blood culture is positive for bacteria, a blood gas profile reveals acidosis, and kidney function tests are abnormal (early in the course of disease). This disease may also alter the results of the following tests: a peripheral smear may demonstrate a low platelet count and destruction of red blood cells, fibrin degradation products are often elevated, a condition that may be associated with a tendency to bleed, CBC and blood tests show a differential in cell count from normal standards—with immature white blood cells generally seen.

Septic patients usually require monitoring in an intensive care unit (ICU). Broad spectrum intravenous antibiotic therapy should be initiated as soon as sepsis is suspected. The number or kind of antibiotics administered may be decreased when the results of blood cultures become available and the causative organism is identified. Thus, a rapid and accurate diagnostic assay is critical, one that can uncover the exact etiological agent(s) responsible, which in turn allows for a more accurate antibiotic therapy regimen, since administration of inappropriate antibiotics can exacerbate the infection. The source of the infection should be discovered, which may require further diagnostic testing on the cells, organs and tissues of a patient. As a precaution, sources such as infected intravenous lines or surgical drains should be removed, and sources such as abscesses should be surgically drained. Supportive therapy with oxygen, intravenous fluids, and medications that increase blood pressure may be required for a good outcome. Dialysis may be necessary in the event of kidney failure, and mechanical ventilation is often required if respiratory failure occurs. The prognosis for septic patients depends on numerous factors, and early diagnosis and antibiotic intervention are critical. The death rate can be as high as 60% for people with underlying medical problems. Mortality is less (but still significant) in individuals without other medical problems.

Sepsis causes significant immune system changes. In response to antigenic challenge, i.e., from bacteria or other pathogens, antigen presenting cells secrete a number of cytokines into the bloodstream, most notably the interleukins IL-1, IL-6 and IL-11 and TNF-alpha. Interleukin-1 (IL-1) is an important part of the inflammatory response, and is secreted by macrophages, monocytes and dendritic cells, among others. Interleukin-6 (IL-6) is a pro-inflamatory cytokine secreted by antigen presenting cells to stimulate an immune response in response to tissue damage, which leads to inflammation. DC produce significant levels of IL-6, which negatively regulates IL-12 production which polarizes the immune system to a Th1 (cell mediated) immune response. Tumor necrosis factor alpha (TNFα) a proinflammatory cytokine that is produced by leukocytes (DC, monocytes and macrophages among others). B and T cells demonstrate specificity for antigens through the B cell receptor (BCR) and the T cell receptor (TCR) respectively. BCRs bind soluble antigens (like diphtheria toxoid). The bound antigen molecules are engulfed into the B cell by receptor-mediated endocytosis. The antigen is digested into fragments which are then displayed at the cell surface by class II histocompatibility molecules. Helper T cells specific for this structure (i.e., with complementary TCRs) bind the B cell and secrete lymphokines that stimulate the B cell to enter the cell cycle and develop, by repeated mitosis, into a clone of cells with identical BCRs; switch from synthesizing their BCRs as integral membrane proteins to a soluble version; and differentiate into plasma cells that secrete these soluble BCRs, which are now called antibodies. CD4+ T cells bind antigenic epitopes that are part of class II histocompatibility molecules. Antigen-presenting cells including B cells, and phagocytic cells like macrophages and dendritic cells all express class II molecules and present antigenic epitopes to other immune cells in this context. The T cells then release lymphokines that attract other cells to the area. The result is inflammation, the accumulation of cells and molecules that attempt to wall off and destroy the antigenic material. Gamma/Delta T cells, like alpha/beta T cells, develop in the thymus. However, they migrate from there into body tissues, especially epithelia (e.g., intestine, skin, lining of the vagina), and don't recirculate between blood and lymph nodes (they represent no more than 5% of the T cells in the blood and are even rarer in lymph nodes). They encounter antigens on the surface of the epithelial cells that surround them rather than relying on the APCs found in lymph nodes. Situated as they are at the interfaces between the external and internal worlds, they may represent a first line of defense against invading pathogens. Their response does seem to be quicker than that of αβ T cells. Curiously, many of the antigens to which γδ T cells respond are found not only on certain types of invaders (e.g., Mycobacterium tuberculosis, the agent of tuberculosis) but also in host cells that are under attack by pathogens.

### Biomarkers for Infection

APC's thus encounter microorganisms and will process various antigens, of which the antigens can be directly identified or isolated from the APC as described above. Contact with bacterial antigens initiates a series of biochemical responses in the APC, each APC having a specific response depending on variables such as the specific APC cell type (B-cell, TH1-cell, TH2-cell, DC, macrophage, monocyte, etc.) and the particular bacterial antigen (for example, commonly LPS and further including those discussed below). These biochemical changes provide methods for identifying the type and severity of a bacterial infection. In addition, the effect the antigen contacted APC have upon other immune cells and organs can also be detected, and provide additional methods for detecting and monitoring an infection in a subject.

Acute phase proteins (APP), also known as acute phase reactants (APR) is the generic name given to a group of approximately 30 different biochemically and functionally unrelated proteins. The levels of acute phase proteins in the serum are either increased (positive acute phase reactants) or reduced (negative acute phase reactants) approximately 90 minutes after the onset of an inflammatory reaction, and particularly in response to a bacterial infection. The more important acute phase proteins are usually glycoproteins. Exceptions are C-reactive protein (CRP) and serum amyloid A protein (SAA).

The APP are produced in response to various cytokines produced by immune cell stimulation. The major inducers of acute phase proteins are IL1, IL6, and TNF-alpha. The two mediators IL1 and IL6 have been used to classify acute phase proteins into two subgroups. Type-1 acute phase proteins are those that require the synergistic action of IL6 and IL1 for maximum synthesis. Examples of Type-1 proteins are C-reactive protein, serum amyloid A and alpha-1 acid glycoprotein. Type-2 acute phase proteins are those that require IL6 only for maximal induction. Examples of Type-2 proteins are fibrinogen chains, haptoglobin, and alpha-2-Macroglobulin. Expression of genes encoding Type-2 acute phase proteins is suppressed rather than being enhanced frequently by IL1. Additive, synergistic, co-operative, and antagonistic effects between cytokines and other mediator substances influencing the expression of acute phase proteins do occur and have been observed in almost all combinations. Many cytokines also show differential effects, inducing the synthesis of one or two acute phase proteins but not others. For example, Activin A induces a subset of acute phase proteins in HepG2 cells. Bacterial lipopolysaccharides and several cytokines (mainly IL1, IL6 and TNF-alpha but also LIF, CNTF, oncostatin M, IL11, and cardiotrophin-1) are involved in the induction of SAA synthesis and some of these cytokines act synergistically. IL1 and also IFN-gamma reduce some of the effects of IL6. Some of the effects of IL2 and IL6 are antagonized by TGF-beta. The combined action of two or even more cytokines may produce effects that no factor on its own would be able to achieve. In cultured HepG2 hepatoma cells IL1, IL6, TNF-alpha and TGF-beta induce the synthesis of antichymotrypsin and at the same time repress the synthesis of albumin and AFP (alpha-Fetoprotein). The synthesis of fibrinogen is induced by IL6 and this effect is, in turn, suppressed by IL1-alpha, TNF-alpha or TGF-beta-1. The increased synthesis of Haptoglobin mediated by IL6 is suppressed by TNF-alpha. Insulin inhibits the synthesis of some negative acute phase proteins (prealbumin, transferrin, and fibrinogen), in HepG2 hepatoma cells.

The fact that different patterns of cytokines are involved in systemic and localized tissue damage in response to infection is supported by observations with knock-out mice for IL1 and IL6. Inflammatory acute phase response after tissue damage or infection is severely compromised in IL6 knock-out mice, but only moderately affected after challenge with bacterial lipopolysaccharides. In the absence of IL6, the induction of acute phase proteins is dramatically reduced in response to chemical challenge with turpentine but that parameters are altered to the same extent both in wild-type and IL6-deficient mice following injection of bacterial lipopolysaccharides. These mice, however, produce three times more TNF-alpha than wild-type controls. A normal acute phase reaction is observed to both turpentine and bacterial lipopolysaccharide challenge in TNF-beta knock-out mice. IL1-beta knock-out mice, on the other hand, show a normal response to bacterial lipopolysaccharides, suggesting that IL1-beta is not essential for the in vivo systemic response to bacterial lipopolysaccharides or that its role can be fulfilled by other cytokines with overlapping activities.

Acute phase proteins regulate immune responses, function as mediators and inhibitors of inflammation, act as transport proteins for products generated during the inflammatory process (the heme-binding protein hemopexin, and haptoglobin), and/or play an active role in tissue repair and remodeling. At least some acute phase proteins might constitute an inducible system of factors protecting against cell death by apoptosis. For example, alpha1-acid glycoprotein and alpha1-antitrypsin activate the major executioners of apoptosis, caspase-3 and caspase-7. Some of the acute phase proteins behave like cytokines. C-reactive protein, for example, activates macrophages, but appears to inhibit CD14+ DC maturation and differentiation. Other acute phase proteins influence the chemotactic behavior of cells. Some acute phase proteins possess antiproteolytic activity and presumably block the migration of cells into the lumen of blood vessels thus helping to prevent the establishment of a generalized systemic inflammation. A failure to control uncontrolled acute phase reactions eventually has severe pathological consequences, such as systemic inflammatory response syndrome (SIRS). The co-ordinated expression of many acute phase proteins as a direct consequence of the activities of several cytokines can be explained, at least in part, by the fact that the regulatory sequences of the genes encoding these acute phase proteins contain so-called cytokine response elements (for example, IL6RE as an IL6-specific element). These elements are recognized specifically by transcription factors that mediate the activity of these genes in a cell- and/or tissue-specific manner.

Acute phase proteins are synthesized predominantly in the liver with each hepatocyte possessing the capacity to produce the entire spectrum of these proteins. These acute phase proteins include: alpha-1 acid glycoprotein; alpha-1 antichymotrypsinogen; alpha-1 antitrypsin; alpha-2 antiplasmin; alpha-2-macroglobulin; antithrombin-3; the complement proteins (including C1, C2, C4, C4 binding protein, C5, C9 and Factor-B); C-reactive protein; ceruloplasmin; Factor VIII; ferritin; fibrinogen; fibronectin; haptoglobin; heme oxygenase; hemopexin; heparin cofactor-2; kallikreins; LPS binding protein; manganese superoxide dismutase; mannose-binding protein; plasminogen; plasminogen activator inhibitor-; prothrombin; serum amyloid A; serum amyloid-P; von Willebrand factor; and IL1ra (IL1 receptor antagonist). Following stimulation of single hepatocytes within individual lobules one observes a stimulation of further hepatocytes and this process continues until almost all hepatocytes produce these acute phase proteins and release them into the circulation.

The various acute phase proteins differ markedly in the rise or decline of their plasma levels and also in their final concentrations. The elevated serum concentrations of certain acute phase proteins are of diagnostic relevance and also of prognostic value. Acute phase responses generate a characteristic serum protein profile, which can be used to monitor the stages of infection as well as the types of pathogens that are causing the response. Their measurement allows inflammatory processes to be distinguished from functional disturbances with similar or identical clinical pictures. Under normal circumstances an acute phase response is not observed with functional disturbances that are not the result of an inflammatory process, thereby allowing the differentiation between failure of function and organic disease. Some acute phase reactions are observed also in chronic disorders such as rheumatoid arthritis and chronic infections while malignant diseases are almost invariably associated with an acute phase reaction. There are many diseases in which the rise in the synthesis of acute phase proteins parallels the degree and progression of the inflammatory processes, particularly in patients with bacterial infections.

One of the landmark studies involving sepsis was the PROWESS Project (Recombinant Human Activated Protein C Worldwide Evaluation in Severre Sepsis), which was a Phase III randomized double blind placebo controlled multicenter trial conducted in patients with severe sepsis (see, Kinasewitz et al., Critical Care, 8:2, 2004). In the study, nineteen biomarkers of sepsis, specific for coagulation activation, anticoagulation, fibrinolysis, endothelial injury, and inflammation were analyzed to determine baseline values and their change over time, in view of different causative agents of sepsis. The nineteen biomarkers of sepsis include: D-dimer; IL-6; Protein C; antithrombin; Protein S; prothrombin time; activated partial prothromboplastin time; platelets; prothrombin fragment 1.2; thrombin-antithrombin complex; thrombin-activatable fibrinolysis inhibitor; alpha-2 antiplasmin; plasminogen; plasminogen activator inhibitor; soluble thrombomodulin; IL-1 beta; IL-10; IL-8; and tumor necrosis factor alpha. The study applied different models of infection, including Gram-negative or Gram positive or mixed bacterial infection or fungal infection. The result of the study indicated that subject response to sepsis included host inflammatory and coagulopathic responses, which were remarkably similar in Gram-positive, Gram-negative and fungal sepsis. A more refined look at the individual immune cells and processes reveals organismal differences.

Dendritic cells (DCs) play a key role in critical illness and are depleted in spleens from septic patients and mice. Efron P A, et al. (J Immunol. 2004 Sep. 1; 173(5):3035-43) characterized the systemic loss of dendritic cells in murine lymph nodes during polymicrobial sepsis. They analyzed the phenotype of DCs and Th cells present in the local (mesenteric) and distant (inguinal and popliteal) lymph nodes of mice with induced polymicrobial sepsis (cecal ligation and puncture method). Flow cytometry and immunohistochemical staining demonstrated that there was a significant local (mesenteric nodes) and partial systemic (inguinal, but not popliteal nodes) loss of DCs from lymph nodes in septic mice, and that this process was associated with increased apoptosis. This sepsis-induced loss of DCs occurred after CD3(+)CD4(+) T cell activation and loss in the lymph nodes, and the loss of DCs was not preceded by any sustained increase in their maturation status. In addition, there was no preferential loss of either mature/activated (MHCII(high)/CD86(high)) or immature (MHCII(low)/CD86(low)) DCs during sepsis. However, there was a preferential loss of CD8(+) DCs in the local and distant lymph nodes. They concluded a loss of DCs in lymphoid tissue, particularly CD8(+) lymphoid-derived DCs, may contribute to the alterations in acquired immune status that frequently accompany sepsis.

Another group studied additional factors associated with the septic response as predictors of mortality in animal models (cecal ligation and puncture) of sepsis. Heuer et al., (Crit Care Med. 32:7, 2004), studied biomarkers for coagulation and inflammation in response to sepsis. Biomarkers for sepsis included: blood glucose; protein C; blood colony forming unit; D-dimer; apolipoprotein A1; beta-2 microglobulin; C-reactive protein; epidermal growth factor; endothelin-1; eotaxin; Factor VII; fibroblast growth factor-9; basic fibroblast growth factor; fibrinogen; granulocyte chemotactic protein-2; granulocyte-macrophage colony stimulating factor; growth hormone; glutathione S-transferase; gamma interferon; IgA; IL-10; IL-11; IL-12p70; IL-17; IL-18; IL-1beta; IL-2; IL-3; IL-4; IL-5; IL-6; IL-7; insulin; gamma interferon inducible protein 10; KC; leptin; leukemia inhibitory factor; lymphotactin; monocyte chemoattractant protein-1/JE; monocyte chemoattractant protein-3; monocyte chemoattractant protein-5; macrophage colony stimulating factor; macrophage derived chemokine; macrophage inflammatory protein-1 alpha; macrophage inflammatory protein-1 beta; macrophage inflammatory protein-1 gamma; macrophage inflammatory protein-2; macrophage inflammatory protein-3 beta; myoglobin; oncostatin M; RANTES; stem cell factor; aspartate amino transferase; tissue inhibitor metalloproteinase-1; tumor necrosis factor-alpha; tissue factor; thrombopoietin; vascular cell adhesion molecule-1; vascular endothelial growth factor; and von Willebrand factor. The authors concluded that early decrease in protein C concentration predicts poor outcome in a rat sepsis model, and that increases in the CXC chemokines macrophage inflammatory protein-2 and KC also precede poor outcome.

Advances in diagnostic detection of infection and sepsis through soluble biomarker detection and quantitative cellular measurements thus provide the basis for improved diagnostic techniques. Davis B H. (Expert Rev Mol Diagn. 2005 March; 5(2): 193-207) studied various approaches to infection/sepsis detection. Procalcitonin offered an enhanced diagnostic distinction between bacterial and viral etiologies. Neutrophil CD64 measurements offered superior sensitivity and specificity to conventional laboratory assessment of sepsis. Neutrophil CD64 expression is negligible in the healthy state. However, it increases as part of the systemic response to severe infection or sepsis. The combination of cellular proteomics, as in the case of neutrophil CD64 quantification, and selected soluble biomarkers of the inflammatory response, such as procalcitonin or triggering receptor expressed on myeloid cells (TREM)-1, provides improved methods in the diagnosis and therapeutic monitoring of infection and sepsis. In addition, Nupponen et al., (Pediatrics, 108:1 Jul. 2001), assessed circulating IL-8 and neutrophil CD11b expression as markers for early-onset infection in human neonates. The authors found that CD11b and IL-8 levels are superior to CRP levels in the detection of systemic infection at its early stages. This finding was confirmed by Turunen et al. (Pediatric. Res. 57(2):270-275 (2005), who described the increased CD-11b density on circulating phagocytes as an early sign of late-onset sepsis in neonates. In addition, Schieven, G. L., H. de Fex, et al. (Antioxid Redox Signal 4(3): 501-7 (2002) discovered that hypochlorous acid activates tyrosine phosphorylation signal pathways leading to calcium signaling and TNFalpha production in neutrophils. Hypochlorous acid is an important oxidizing agent produced by neutrophils to aid in defense against pathogens. Although hypochlorous acid is known to cause tissue damage due to its cytotoxicity, the effect of this oxidizing agent on signal transduction by cells of the immune system and its effects on their responses are not well understood. Hypochlorous acid was found to induce cellular tyrosine phosphorylation in both T and B lymphocytes, activate the ZAP-70 tyrosine kinase, and induce cellular calcium signaling in a tyrosine kinase-dependent manner. These signaling events also occurred in T cell lines that did not express the T-cell receptor, indicating the ability of hypochlorous acid to bypass normal receptor control. Hypochlorous acid induced tumor necrosis factor-alpha production in peripheral blood mononuclear cells in a tyrosine kinase-dependent manner. These results suggest that hypochlorous acid may contribute to inflammatory responses by activating signal pathways in cells of the immune system. As such, hypochlorous acid production and the various proteins in the downstream signaling pathways provide APC biomarkers for inflammation and sepsis.

Lainee P, Crit Care Med. 2005 April; 33(4):797-805 found that delayed neutralization of interferon-gamma prevents lethality in primate Gram-negative bacteremic shock. The study investigated whether delayed administration of a novel anti-human interferon-gamma monoclonal antibody could improve outcome and reduce organ injury in a lethal model of Escherichia coli bacteremia, when administered after the onset of shock. In a primate model of E. coli bacteremic shock, delayed neutralization of interferon-gamma after the onset of shock improved survival and attenuated the pathologic changes associated with the development of organ dysfunction. These findings suggest that interferon-gamma blockade represents a potentially effective mode of late intervention in lethal septic shock. In addition, monitoring of interferon-gamma provides a diagnostic for disease progression.

Gibot S, et al., (Crit Care Med. 2005 April; 33(4):792-6) measured the time-course of sTREM (soluble triggering receptor expressed on myeloid cells)-1, procalcitonin, and C-reactive protein plasma concentrations during sepsis. Soluble TREM-1 concentrations were significantly lower at admission in nonsurvivors than in survivors (94 [30-258] vs. 154 [52-435] pg/mL, p=0.02), whereas PCT levels were higher among nonsurvivors (19.2 [0.3-179] vs. 2.4 (0-254) pg/mL, p=0.001). CRP levels did not differ between the two groups of patients. Plasma PCT and CRP decreased during the 14-day period of study in both survivors and nonsurvivors. Conversely, sTREM-1 plasma concentrations remained stable or even increased in nonsurviving patients and decreased in survivors. An elevated baseline sTREM-1 level was found to be an independent protective factor with an odds of dying of 0.1 (95% confidence interval, 0.1-0.8). A progressive decline of plasma sTREM-1 concentration indicates a favorable clinical evolution during the recovery phase of sepsis. In addition, baseline sTREM-1 levels and changes thereto provide a method of predicting outcome of septic patients.

Ahmed Z, et al. (J Coll Physicians Surg Pak. 2005 March; 15(3):152-6) studied the diagnostic value of C-reactive protein and hematological parameters in neonatal sepsis. One hundred neonates having clinical features of sepsis and 100 normal asymptomatic neonates were evaluated with a set of investigations. C-reactive protein (CRP), erythrocyte sedimentation rate, total leukocyte count, absolute neutrophil count (ANC), immature neutrophils to total neutrophil count ratio (I/T ratio), thrombocytopenia, degenerative changes in the neutrophils and gastric aspirate cytology (GAC) for polymorphs were used for diagnosis of neonatal sepsis. CRP was positive in 24/28 (85.7%) of group-A (proven sepsis) and 58/72 (80.5%) of group-B (probable sepsis) and had a specificity of 95%. ANC was the second most sensitive test having sensitivity of 71.4% for group-A and 63.9% for group-B and 88% specificity. For group-A, sensitivity of GAC for polymorphs and platelet count was 71.4% and 64.3% respectively. The sensitivity, specificity and predictive values (PV) of the individual tests and different tests combination was also calculated for group-A and B. A set of investigations including CRP, TLC, ANC, thrombocytopenia, cytoplasmic vacuolization in the neutrophils and GAC for polymorphs are highly sensitive in detection of culture negative cases of neonatal sepsis.

Sierra R, et al. (Intensive Care Med. 2004 November; 30(11):2038-45) investigated C-reactive protein as a marker of sepsis and as an early indicator of infection in patients with systemic inflammatory response syndrome. Serum C-reactive protein concentration was measured within the first 24 h of SIRS onset. Healthy subjects, AMI and non-infectious SIRS patients showed lower C-reactive protein median values ([(0.21 [95% confidence intervals (95% CI), 0.21-0.4] mg/dl, 2.2 [95% CI, 2.1-4.9] mg/dl and 1.7 [95% CI, 2.4-5.5] mg/dl, respectively) than patients with sepsis (18.9 [95% CI, 17.1-21.8]), p<0.001. The presence of severe sepsis (r(s)=0.27; p=0.03), SOFA score (r(s)=0.25; p=0.03) and arterial lactate (r(s)=0.24; p=0.04) correlated significantly with C-reactive protein concentrations in sepsis cases. The best threshold value for C-reactive protein for predicting sepsis was 8 mg/dl (sensitivity 94.3%, specificity 87.3%). Ng, et al. (Pediatr Res. 2004 November; 56(5):796-803) evaluated the diagnostic utilities of C-reactive protein and neutrophil CD64 expression for the identification of early-onset clinical infection, pneumonia and sepsis in term infants. They found neutrophil CD64 was a sensitive diagnostic marker for the identification of early-onset clinical infection and pneumonia in term newborns.

Mattner J, et al., (Nature. 2005 Mar. 24; 434(7032):525-9) studied activation of NKT cells by exogenous and endogenous glycolipid antigens during microbial infections. CD1d-restricted natural killer T (NKT) cells are innate-like lymphocytes that express a conserved T-cell receptor and contribute to host defence against various microbial pathogens. However, their target lipid antigens have remained elusive. There is strong evidence for microbial, antigen-specific activation of NKT cells against Gram-negative, lipopolysaccharide (LPS)-negative alpha-Proteobacteria such as Ehrlichia muris and Sphingomonas capsulata. Glycosylceramides isolated from the cell wall of Sphingomonas, serve as direct targets for mouse and human NKT cells, controlling both septic shock reaction and bacterial clearance in infected mice. In contrast, Gram-negative, LPS-positive Salmonella typhimurium activates NKT cells through the recognition of an endogenous lysosomal glycosphingolipid, iGb3, presented by LPS-activated dendritic cells. Glycosylceramides are an alternative marker to LPS for innate recognition of the Gram-negative, LPS-negative bacterial cell wall. APC responses to glycosylceramides provide biomarkers for these pathogens.

Persistent elevation of high mobility group box-1 protein (HMGB1) in patients with severe sepsis and septic shock. Cytokine levels were measured at five time points during the first week after admission and were correlated to Acute Physiology and Chronic Health Evaluation II and Sepsis-related Organ Failure Assessment scores. Two HMGB1 assays were used. Both demonstrated delayed kinetics for HMGB1 with high levels on inclusion that remained high throughout the study period. Serum concentration at 144 hrs, the last sampling point, was 300 times higher, 34,000+/−76,000 pg/mL (mean +/−sd), than any of the other cytokines. This study, however, found no predictable correlation between serum levels of HMGB1 and severity of infection. Levels of interleukin-6, interleukin-8, interleukin-10, and tumor necrosis factor-alpha correlated significantly with severity of disease, and all were significantly higher in patients with septic shock compared with those with severe sepsis. Levels for HMGB1 remained high in the majority of patients up to 1 week after admittance, indicating that HMGB1 is a downstream and late mediator of inflammation. HMGB1 thus provides a biomarker for sepsis.

el-Sameea E R, et al. (Egypt J Immunol. 2004; 11(1):91-102) evaluated natural killer cells as diagnostic markers of early onset neonatal sepsis, compared with C-reactive protein and interleukin-8. The study aimed at revealing the role played by the NK cells in neonatal sepsis and evaluating the sensitivity of NK cell number and cytotoxicity as diagnostic markers in infants with suspected early neonatal sepsis compared with the circulating cytokine IL-8 and CRP levels. All samples of peripheral blood lymphocytes were subjected to determination of CD16 and CD56 positive cells using flow cytometry and NK cytotoxicity using the standard 4h 51Cr release assay. Sera were separated to measure IL-8 using ELISA. The median CRP value was significantly higher in sepsis group (88 mg/L; range: 17-159 mg/L) compared with that in non-septic group (15.4 mg/L; range: 7.6-23.2 mg/L, p<0.001) only 12-60 h after admission. On the other hand, newborns in the sepsis group had significantly higher serum levels of IL-8 (median 310 pg/mL; range: 37-583 pg/ml) at study entry than that in the non septic group (median 63 pg/mL; range: 32-94 pg/ml, P<0.001). On admission, the NK activity, rather than the number of CD16 and CD56 positive cells was much affected where NK cytotoxicity was significantly lower in sepsis group (3.4+/−2.1%, range 0.9-7%) than that of the nonseptic group (18.3+/−6.7%: range 10.7-25.3%, p<0.01) and healthy neonates (23.8+/−4.7%: range 12.2-32.3%, p<0.001). Defective NK cell activity rather than NK cell number plays an important role in susceptibility to early onset neonatal sepsis. Evaluation of NK cytotoxicity as a marker in early diagnosis of neonatal sepsis reveals that the sensitivity, specificity and predictive values of reduced NK cytotoxicity (10% killing) was higher than both of CRP and IL-8, either individually or in combination. Additionally, reduced NK cytotoxicity showed high correlation with the severity and outcome of neonatal sepsis, permitting APC responses to be used as a prognostic indicator of sepsis.

Marsik C, et al. (Clin Immunol. 2005 March; 114(3):293-8) studied expression of the signaling receptor (GP130) on protein and molecular level in endotoxemia patients. Interleukin 6 (IL-6) performs a prominent role during sepsis. To examine the molecular regulation of IL-6, IL-6 receptor, and signaling receptor gp130 during endotoxemia, nine healthy young volunteers received a bolus injection of lipopolysaccharide (LPS) on day 1 and saline on day 2 in a double blind, randomized, placebo-controlled trial. LPS enhanced IL-6 release 300-fold. IL-6 mRNA expression was not significantly altered in blood samples at any time after LPS infusion in vivo, while incubation of whole blood with 50 pg/ml LPS up-regulated IL-6 mRNA levels 8000- to 50,000-fold in vitro. LPS infusion increased synthesis of gp130 mRNA 5.5-fold compared to baseline at 4 h (P<0.05), while no significant change was observed in the placebo period (P=0.001 between groups). LPS increased the percentage of gp130 positive neutrophils gp130 700% over baseline at 8 h (P<0.01 versus baseline and placebo). IL-6 receptor levels were not significantly altered by low-grade endotoxemia. Endotoxemia up-regulates gp130 expression in vivo and in vitro. APC expression of gp130 is thus a biomarker for endotoximia and sepsis.

Morgenthaler N G, et al. (Crit Care. 2005 February; 9(1):R37-45) investigated pro-atrial natriuretic peptide is a prognostic marker in sepsis. The prognostic value of mid-regional pro-atrial natriuretic peptide (ANP) levels was compared with that of the Acute Physiology and Chronic Health Evaluation (APACHE) II score and with those of various biomarkers (i.e. C-reactive protein, IL-6 and procalcitonin), detected in EDTA plasma from patients using a sandwich immunoassay. The median pro-ANP value in the survivors was 194 pmol/l (range 20-2000 pmol/l), which was significantly lower than in the nonsurvivors (median 853.0 pmol/l, range 100-2000 pmol/l; P<0.001). On the day of admission, pro-ANP levels, but not levels of other biomarkers, were significantly higher in surviving than in nonsurviving sepsis patients (P=0.001). In a receiver operating characteristic curve analysis for the survival of patients with sepsis, the area under the curve (AUC) for pro-ANP was 0.88, which was significantly greater than the AUCs for procalcitonin and C-reactive protein, and similar to the AUC for the APACHE II score.

Lowry S F, et al. (Surg Infect (Larchmt). 2004 Fall; 5(3):261-8) studied static and dynamic assessment of biomarkers in surgical patients with severe sepsis. Severe sepsis, defined as a systemic inflammatory response to infection associated with acute organ dysfunction, is common among surgical patients and is a major cause of morbidity and mortality. Severe sepsis has been associated with changes in inflammatory and hemostatic biomarkers. In patients undergoing surgical procedures there may be additional stimulation of cytokine release and activation of the coagulation system. The study characterized the baseline differences in biomarkers between surgical and non-surgical patients. In addition, they assessed the dynamic changes in biomarkers and coagulation parameters in surgical patients with severe sepsis. Biomarkers and coagulation parameters available for analysis were D-dimer, interleukin-6 (IL-6), protein C activity, protein S activity, anti-thrombin III (ATIII), activated partial thromboplastin time (aPTT), and prothrombin time (PT) and platelet count. Surgical patients with severe sepsis appeared to have a higher severity of illness at baseline as demonstrated by derangements in biomarkers and coagulation markers compared to non-surgical patients. Surgical patients treated with drotrecogin alfa (activated) showed reduced D-dimer concentrations and a more rapid increase in protein C concentrations during the infusion period.

O'Connor E, et al., (Anaesth Intensive Care. 2004 August; 32(4):465-70) studied serum procalcitonin and C-reactive protein as markers of sepsis and outcome in patients with neurotrauma and subarachnoid haemorrhage. Sixty-two patients were followed for 7 days. Serum PCT and CRP were measured on days 0, 1, 4, 5, 6 and 7. Seventy-seven percent of patients with traumatic brain injury and 83% with subarachnoid haemorrhage developed SIRS or sepsis (P=0.75). Baseline PCT and CRP were elevated in 35% and 55% of patients respectively (P=0.03). There was a statistically non-significant step-wise increase in serum PCT levels from no SIRS (0.4+/−0.6 ng/ml) to SIRS (3.05+/−9.3 ng/ml) to sepsis (5.5+/−12.5 ng/ml). A similar trend was noted in baseline PCT in patients with mild (0.06+/−0.9 ng/ml), moderate (0.8+/−0.7 ng/ml) and severe head injury (1.2+/−1.9 ng/ml). Such a gradation was not observed with serum CRP. There was a non-significant trend towards baseline PCT being a better marker of hospital mortality compared with baseline CRP (ROC-AUC 0.56 vs 0.31 respectively).

Arredouani M S, et al., (Immunology. 2005 February; 114(2):263-71) studied haptoglobin's ability to dampen endotoxin-induced inflammatory effects. Haptoglobin, an acute-phase protein produced by liver cells in response to interleukin-6 (IL-6), can modulate the inflammatory response induced by endotoxins. Haptoglobin has the ability to selectively antagonize lipopolysaccharide (LPS) effects in vitro by suppressing monocyte production of tumor necrosis factor-alpha, IL-10 and IL-12, while it fails to inhibit the production of IL-6, IL-8 and IL-1 receptor antagonist. In two animal models of LPS-induced bronchopulmonary hyperreactivity and endotoxic shock, haptoglobin knockout mice were more sensitive to LPS effects compared to their wild-type counterparts. Haptoglobin thus appears to regulate monocyte activation following LPS stimulation. The increase in haptoglobin levels during an acute-phase reaction may generate a feedback effect which dampens the severity of cytokine release and protects against endotoxin-induced effects. Thus haptoglobin is a marker for sepsis and correlates with disease severity.

Tsujimoto H, et al. (Shock. 2005 January; 23(1):39-44) studied neutrophil elastase, MIP-2, and TLR-4 expression as markers during human and experimental sepsis. Highly activated neutrophils play a critical role in mediating organ injury in sepsis by releasing neutrophil elastase (NE). Toll-like receptors (TLRs) play an important role in the host defense against invading microbes, and their signaling pathway is critical to the activation of the proinflammatory response. The study investigated the relationships among chemokine (MIP-2), TLR-4, and NE expression in human sepsis and murine peritonitis (CLP). TLR-4 expression on monocytes/macrophages was examined in patients with sepsis and in murine peritonitis and was markedly increased in both populations. LPS-induced MIP-2 production by bronchoalveolar cells and liver mononuclear cells in mice with peritonitis was also significantly increased compared with sham-operated mice. Pretreatment of the macrophage cell line, RAW 264.7 cells, with a NE inhibitor before their exposure to LPS resulted in a significant dose-dependent decrease in MIP-2 production, which was comparable to that seen following pretreatment with TLR-4 antibody. Furthermore, NE and LPS both up-regulated TLR-4 expression on human peripheral blood monocytes. Thus, chemokine-induced recruitment of neutrophils in sepsis may result in further increased chemokine production and increased expression of TLR-4. Neutrophil-derived NE may be associated with increased expression of monocyte/macrophage TLR-4, thereby serving as a positive feedback loop for the inflammatory response, and a biomarker for the same among the different cell populations.

van Rossum A M, et al. (Lancet Infect Dis. 2004 October; 4(10):620-30) investigated procalcitonin as an early marker of infection in neonates and children. The study found that procalcitonin is an excellent marker for severe, invasive bacterial infection in children. However, the use of procalcitonin in the diagnosis of neonatal bacterial infection is complicated, but may result in a higher specificity than C-reactive protein. In addition, procalcitonin was shown to correlate with severity of disease (urinary tract infections and sepsis), making it useful as a prognostic biomarker for infection and sepsis.

Turunen R, et al. (Pediatr Res. 2005 February; 57(2):270-5) studied the increased CD11b-density on circulating phagocytes as an early sign of late-onset sepsis in extremely low-birth-weight infants. Late-onset hospital-acquired sepsis is common in extremely low birth-weight (<1000 g) (ELBW) infants. The diagnosis is difficult since, at early stages of sepsis, routine laboratory tests are neither specific nor sensitive. In term infants with sepsis, neutrophil surface expression of CD11b/CD18, a beta2-integrin, is significantly increased. Increased CD11b/CD18 density on blood neutrophils and monocytes was found to be an early sepsis marker in ELBW infants. Neutrophil and monocyte CD11b/CD18 expression was determined by flow-cytometry. CD11b expression gradually increased during the three days preceding sampling for blood culture. At the day of sampling, median expression of CD11b in neutrophils and monocytes was higher in the infected group than in the control group. For neutrophils the sensitivity and specificity were 1.00 and 0.56, respectively, and for monocytes, 0.86 and 0.94, respectively.

Ashare A, et al. (Am J Physiol Lung Cell Mol Physiol. 2005 April; 288(4):L633-40) investigated the anti-inflammatory response and its association with mortality and severity of infection in sepsis. Using a murine model of sepsis, they found that the balance of tissue pro- to anti-inflammatory cytokines directly correlated with severity of infection and mortality. Sepsis was induced in C57BL/6 mice by cecal ligation and puncture (CLP). Liver tissue was analyzed for levels of IL-1beta, IL-1 receptor antagonist (IL-1ra), tumor necrosis factor (TNF)-alpha, and soluble TNF receptor 1 by ELISA. Bacterial DNA was measured using quantitative real-time PCR. After CLP, early predominance of proinflammatory cytokines (6 h) transitioned to anti-inflammatory predominance at 24 h. The elevated anti-inflammatory cytokines were mirrored by increased tissue bacterial levels. The degree of anti-inflammatory response compared with proinflammatory response correlated with the bacterial concentration. To modulate the timing of the anti-inflammatory response, mice were treated with IL-1ra before CLP. This resulted in decreased proinflammatory cytokines, earlier bacterial load, and increased mortality. The studies showed that the initial tissue proinflammatory response to sepsis is followed by an anti-inflammatory response. The anti-inflammatory phase is associated with increased bacterial load and mortality, and it is the timing and magnitude of the anti-inflammatory response that predicts severity of infection.

Bozza F A, et al. (Shock. 2004 October; 22(4):309-13) investigated the correlation of macrophage migration inhibitory factor (MIF) levels, with fatal outcome in sepsis. MIF levels were compared to interleukin-6 (IL-6) levels in critically ill patients with sepsis and septic shock. They found the median plasma concentrations of MIF and IL-6 were significantly higher in patients with septic shock and in patients with sepsis than in healthy controls, and that MIF levels were significantly different between survivors and nonsurvivors, as were IL-6 levels. Significantly, high plasma levels of MIF (>1100 pg/mL) had a sensitivity of 100% and a specificity of 64% to identify the patients who eventually would evolve to a fatal outcome.

Accordingly, the APP and various cytokines and lymphokines discussed provide protein-based markers for infection and sepsis, provide methods for determining changes in the immune state of a subject in response to infection. While the various immune and organ responses to infection described have focused on protein biomarkers, these proteins necessarily originate from gene expression changes. As such, the various genes encoding the protein biomarkers are themselves detectable, providing nucleic acid-based biomarkers for infection and sepsis. In addition, differentiation or proliferation of leukocytes can indicate infection. Accordingly, these cellular changes as well as detection of changes in gene and protein-based biomarkers permit identification of the stages and severity of infection, e.g., initial stages, intermediate stages, advanced stages, SIRS and sepsis, as well as post-mortem analysis. Samples of fluids from the subject, preferably a human subject are screened or monitored by various nucleic acid or proteomic assays, and preferably in real time as described above, to assay for the presence of, and changes in levels of these biomarkers for infection. Fluids suitable for use include blood, plasma, bone marrow, pericardial, pleural, ascitic, and synovial fluids, cerebrospinal fluids, sputum, urine, lymphatic fluids, and others known in the medical arts.

### APC Pathogen Biosensors

In connection with the detection (qualitative and quantitative) of APP, cytokines, and other biomarkers in the fluids of a subject, the APC themselves can be assayed for contact with specific bacterial antigens to provide a definitive diagnostic for general classes of pathogens (Gram-negative, Gram-positive, spirochete, cocci or rod), particular pathogens (Bacillus; Bordetella; Clostridium; Escherichia; Haemophilus; Helicobacter; Legionella; Listeria; Mycobacterium; Neisseria; Pseudomonas; Salmonella; Shigella; Staphylococcus; Streptococcus; Vibrio; Yersinia and other strains), and particular strains, subtypes or pathogens having specific virulence factors, WMD strains, as well as those strains resulting in nosocomial infections. APC are isolated from a subject and assayed for changes in levels of disease-state biomarkers. The specificity of the gene and protein changes in various APC in response to antigen contact provide a method for determining the type of antigen the APC has contacted.

The genes and proteins in the APC, that are upregulated following contact with a particular antigen (or in response to a biological conditions such as sepsis) provide a set of markers that can be used for more traditional diagnostic assays. For example, the mass spectroscopy signatures of antigen contacted APC provide a rapid and sensitive means to detect exposure to a pathogen. However, the protein markers can also be used to raise antibodies, which can then be used un immunological assays for detecting antigen contact in other individuals or to monitor the course of therapy.

Various pathogenic bacterial strains are discussed below. These pathogens and the various diseases they cause are detectable and identifiable by the APC-based methods described herein, alone and in conjunction with known medial diagnostics for such organisms and diseases. For a more detailed description of particular pathogenic bacterial strains, see, Bergey's Manual of Systematic Bacteriology (2nd Edition), George M. Garrity, Editor-in-Chief, Springer, New York (2001).

Bacillus are rod-shaped, endospore-forming aerobic or facultatively anaerobic, Gram positive bacteria. Although most species of Bacillus are harmless saprophytes two species are considered medically significant: B. anthracis and B. cereus. B. anthracis is the etiological agent for anthrax and key virulence genes of B. anthracis are found on plasmids pXO1 and pXO2. B. cereus is an opportunistic pathogen causing food poisoning manifested by diarrheal or emetic syndromes.

Bordetella are Gram-negative, strictly aerobic coccobacilli. Seven Bordetella species are described in the medical literature. They can broadly be divided into two groups: The first includes B. pertussis, B. parapertussis, and B. bronchisepitca, each of which colonizes the respiratory tracts of mammals. The second group are distantly related to the first group, including B. avium, B. hinzii, B. holmesii, and B. trematum. B. pertussis, B. parapertussis and B. bronchiseptica share many virulence factors and a nearly identical virulence control system encoded by the bvgAS locus. B. pertussis, a strict human pathogen, is the etiologic agent of whooping cough, a highly contagious respiratory disease marked by severe, spasmodic coughing episodes. B. parapertussis causes a milder form of whooping cough in human beings and chronic, nonprogressive pneumonia in sheep. B. bronchiseptica causes chronic respiratory infections in a wide range of animals.

Borrelia burgdorferi is a spirochete which is the causative agent of Lyme disease, the most common tick-borne disease in the United States. The reservoir for the spirochete is the white-footed mouse and the white-tailed deer. Transmission is accomplished by the bite of infected deer ticks. Contact with the tick usually occurs in areas of brush and tall grass. The disease is usually recognized by a distinctive skin lesion, erythema migrans, accompanied by headache, stiff neck, myalgias, arthralgias, fatigue and possible swelling of the lymph nodes. Not all symptoms are seen in every case, complicating diagnosis. While treatable with antibiotics, unrecognized and/or untreated patients may develop meningoencephalitis, myocarditis or even arthritis, particularly in the knees. Lyme disease may be brief or inconsequential, or chronic, persistent and incapacitating. The chronic disease state may resolve in time with or without antibiotic treatment. In a small percentage of cases, there is no resolution even after antibiotic treatment.

Campylobacter pylori has many attributes in common with other campylobacters but it may represent a new genus. It produces abundant quantities of urease, and this property has been used to develop a rapid diagnostic test. The organism is found predominantly beneath the gastric mucus layer that lines the surface epithelium of the stomach. Infection with C. pylori causes an acute histologic gastritis which may become chronic. The bacterium is the etiologic agent in type-B gastritis. Prevalence of the organism in asymptomatic persons appears to be age related. Campylobacter pylori is found commonly in patients with peptic ulcer disease, always in association with chronic gastritis. Eradication of the organism is associated with healing of the gastritis and a lower relapse rate in duodenal ulcer disease.

Clostridium botulinum are anaerobic, Gram-positive spore-forming rods, with the spores being very heat resistant. They can be isolated from the soil and marine environment. Some strain (non-proteolytic) can grow slowly at temperatures down to 3.3° C. They usually will not produce toxins at pH values less than 4.6 and water activity values of less than 0.94. The toxin is one of the most potent toxins known and 10⁻⁶ g is sufficient to kill an adult human. Botulism is difficult to diagnose by clinical symptoms alone as it is often confused with other illnesses such as Guillain-Barré syndrome. The most direct and effective way is to demonstrate the presence of toxin in the serum or feces of the patient or in the food that the patient consumed. Prior to the present assay methods claimed and described, the most and widely used method for detecting toxin is the mouse bioassay. This usually takes 3 days after isolation of the toxin. Clostridium difficile, or C. difficile (a gram-positive anaerobic bacterium), is now recognized as the major causative agent of colitis (inflammation of the colon) and diarrhea that may occur following antibiotic intake. C. difficile infection represents one of the most common hospital (nosocomial) infections around the world. In the United States alone, it causes approximately three million cases of diarrhea and colitis per year. This bacterium is primarily acquired in hospitals and chronic care facilities following antibiotic therapy covering a wide variety of bacteria (broad-spectrum) and is the most frequent cause of outbreaks of diarrhea in hospitalized patients. One of the main characteristics of C. difficile-associated colitis is severe inflammation in the colonic tissue (mucosa) associated with destruction of cells of the colon (colonocytes). The disease involves, initially, alterations of the beneficial bacteria, which are normally found in the colon, by antibiotic therapy. The alterations lead to colonization by C. difficile when this bacterium or its spores are present in the environment. In hospitals or nursing home facilities where C. difficile is prevalent and patients frequently receive antibiotics, C. difficile infection is very common. In contrast, individuals treated with antibiotics as outpatients have a much smaller risk of developing C. difficile infection. Laboratory studies show that when C. difficile colonize the gut, they release two potent toxins, toxin A and toxin B, which bind to certain receptors in the lining of the colon and ultimately cause diarrhea and inflammation of the large intestine, or colon (colitis). Thus, the toxins are involved in the pathogenesis, or development of the disease. Clostridium tetani is a related strain that is the etiological agent of tetanus.

Clostridium perfringens is an anaerobic, Gram-positive, sporeforming rod (anaerobic means unable to grow in the presence of free oxygen). It is widely distributed in the environment and frequently occurs in the intestines of humans and many domestic and feral animals. Spores of the organism persist in soil, sediments, and areas subject to human or animal fecal pollution. Perfringens food poisoning is the term used to describe the common foodborne illness caused by C. perfringens. A more serious but rare illness is also caused by ingesting food contaminated with Type C strains. The latter illness is known as enteritis necroticans or pig-bel disease. The common form of perfringens poisoning is characterized by intense abdominal cramps and diarrhea which begin 8-22 hours after consumption of foods containing large numbers of those C. perfringens bacteria capable of producing the food poisoning toxin. The illness is usually over within 24 hours but less severe symptoms may persist in some individuals for 1 or 2 weeks. A few deaths have been reported as a result of dehydration and other complications. Necrotic enteritis (pig-bel) caused by C. perfringens is often fatal. This disease also begins as a result of ingesting large numbers of the causative bacteria in contaminated foods. Deaths from necrotic enteritis (pig-bel syndrome) are caused by infection and necrosis of the intestines and from resulting septicemia.

Corynebacterium diphtheriae are Gram positive rod, non-sporulating, non-motile, characteristic swelling at one end of bacillus (club shaped), facultative anaerobe, metachromic granules, three biotypes—gravis, mitis, intermedius; produces a toxin. Diphtheria is a toxigenic infection in which the causative organism colonizes the throat and produces a toxin that inhibits protein synthesis in eukaryotic cells. Locally, dead epithelial and white blood cells can cause the formation of a pseudomembrane which has the potential for obstructing the trachea. Unless the airway is cleared or a tracheostomy performed, death may ensue. Death may also occur due to heart, kidney, or other organ damage that occurs when the toxin (not the organism) systemically spreads. The toxin modifies EF2 (elongation factor 2, a factor involved in protein chain elongation) and renders it non-functional. This effectively stops protein synthesis in virtually any organ of the body, although the toxin has a high affinity for the heart and kidney. The toxin, although a single polypeptide chain, is an A-B type toxin in which a part of the toxin (the B domain) binds the toxin to a cell, and part of the toxin enters the cell. After B binds, the molecule is cleaved and A enters the cell. The A domain is an enzyme that has ADP-ribosylating activity and adds the ADP-ribose from NAD to a modified histidine residue, diphthamide, in EF2. The addition of the ADP-ribose group essentially inactivates EF2. The toxin gene has been shown to be part of the genome of a temperate phage that can infect Corynebacteria. After lysogenization, the organism becomes virulent and a potential toxin-producer. Toxin is produced in vivo in response to a low iron concentration.

Enterococcus faecalis (formerly Streptococcus faecalis and also known as as nonhemolytic streptococci, gamma hemolytic streptococci, enterococcus, group D streptococci, vancomycin-resistant enterococcus (VRE)) are Gram-positive cocci, facultatively anaerobic, and occur singly, in pairs or short chains. No hemolysis is observed on blood agar after 24 hours (may see alpha hemolysis after 48 h). The bacteria is a normal inhabitant of intestinal tract and female genital tract, and is occasionally associated with urinary tract infection, bacteremia and bacterial endocarditis. The bacteria is newly recognized as a nosocomially transmitted pathogen and is one of the 3rd most common organisms recovered from nosocomial infections, accounting for 10% of nosocomial infections, 9% of bacteremia infections, 16% urinary tract infections and 5-15% of cases of bacterial endocarditis.

Pathogenic E. coli cause various diseases in humans, including several types of diarrhea, urinary tract infections, sepsis, and meningitis. E. coli strains that cause human diarrhea of varying severity have been divided into six major categories: enterotoxigenic E. coli (ETEC), enteroinvasive E. coli (EIEC), enteropathogenic E. coli (EPEC), enterohemorrhagic E. coli (EHEC), enteroaggregative E. coli (EAEC), and diffusely adherent E. coli (DAEC). Urinary tract infections (UTIs) are the most common extraintestinal E. coli infections and are caused by uropathogenic E. coli (UPEC). In addition, E. coli is the most common Gram-negative bacterium that causes meningitis, particularly during the neonatal period. The pathotype responsible for meningitis and sepsis is called meningitis-associated E. coli (MNEC). The DAEC category of E. coli is defined by the presence of a characteristic, diffuse pattern of adherence to HEp-2 cell monolayers. Two subclasses of DAEC strains exist: diffusely adhering enteropathogenic E. coli (DA-EPEC) harboring a LEE island, and DAECs expressing adhesins of the Afa/Dr family. The EAEC characteristics include lack of secretion of the enterotoxigenic E. coli heat-liable or heat-stable enterotoxins, and adherence to HEp-2 cells in an aggregative (AA) pattern recognized by the distinctive ‘stacked brick’ autoagglutination of the bacteria either on the surface of the HEp-2 cells or on the glass substratum. EAEC strains are heterogeneous but the majority harbor a member of conserved family of virulence plasmids encoding the adhesion factors AAF and Dispersin, and the toxins EAST1, Pet, Pic and ShET1. The EHEC strains have the following characteristics: a locus for enterocyte effacement (LEE), and the ability to produce Shiga toxins.

Major virulence factors in EHEC strains include the adherence factors Efa-1/LifA, Intimin, Paa and ToxB. Toxins include Hemolysin and Stx. The EIEC strains have the following characteristics: Most of the pathogenic E. coli strains remain extracellular, but EIEC is an intracellular pathogen. The virulence factors in EIEC are virtually identical to those in Shigella species. Dysentery caused by EIEC is clinically indistinguishable from that caused by members of the Shigella species using traditional methods of diagnosis, but can be detected using the present methods. EIEC possesses the biochemical profile of E. coli, yet with the genotypic or phenotypic characteristics of Shigella spp. EIEC contains large plasmids that are functionally interchangeable and share significant degrees of DNA homology with the plasmid described in S. flexneri. An important aspect of Shigella pathogenesis is the extremely low ID50. The ID50 for S. flexneri, S. sonnei, and S. dysenteriae is approximately 5000 organisms. In contrast, at least 108 EIEC must be ingested to produce disease. The EPEC strains have the following characteristics: they produce a histopathology on the intestinal epithelium known as the attaching and effacing (A/E) lesion, and display an inability to produce Shiga toxins. A typical EPEC strain carries a large virulence plasmid (the EPEC adhesion factor (EAF) plasmid) that allows them to produce bundle-forming pili and attach to epithelial cells in a characteristic pattern termed localized adherence (LA), denoting the presence of clusters or microcolonies on the surface of host cells. The LA pattern is characteristic only of EPEC strains of E. coli and therefore has been used widely as a diagnostic tool. Major virulence factors in EPEC strains include the adherence factors BFP, Intimin, Lymphostatin/LifA and Paa and the toxins CDT and EAST1. ETEC strains have the following characteristics: they are distinguished from other E. coli pathotypes their by production of enterotoxins LT (heat-labile enterotoxin) and ST (heat-stable enterotoxin). ETEC strains might express only an LT, only an ST, or both LTs and STs. Producing one or more colonization factors (CFs) that mediate attachment to intestinal mucosal surfaces, is a central step in ETEC virulence. MNEC strains have the following characteristics: E. coli strains possessing the K1 capsular polysaccharide are predominant (approximately 80%) among isolates from neonatal E. coli meningitis and that most of these K1 isolates are associated with a limited number of O types (e.g., O-18, O-7, O-1). These strains generally follow a natural route of infection (e.g., oral), gut colonization and translocation, dissemination to deeper tissues, and a level of bacteremia necessary prior to penetration of the blood-brain barrier. E. coli K1 invades brain microvascular endothelial cells (BMECs) via a zipper mechanism and transmigrates through BMECs in an enclosed vacuole without intracellular multiplication. Major virulence factors in MNEC strains include the adherence factors S fimbriae and the toxin. CNF-1. UPEC strains have the following characteristics: A subgroup of extraintestinal pathogenic E. coli (ExPEC), they typically carry large blocks of genes, called pathogenicity islands, not found in fecal isolates. UPEC can invade and replicate within uroepithelial cells. Major virulence factors in UPEC strains include the adherence factor Dr adhesions and the toxins CNF-1 and Hemolysin.

Francisella tularensis is a small Gram-negative aerobic bacillus with two main serotypes: Jellison Type A and Type B. Type A is the more virulent form. If infection is suspected, diagnosis can be made based on serological assays since F. tularensis is difficult to culture on standard media. Agglutination titers can be performed following the first week of infection and reach a peak during the 4-8 weeks. Infected individuals are normally placed on a regimen of streptomycin or gentamycin for 10-14 days. Beta-lactams are generally ineffective due to beta-lactamase activity.

Haemophilus is a Gram-negative coccobacilli. The name of the genus Haemophilus (meaning blood loving) refers to the dependence of the organism on heme-related molecules for growth under aerobic conditions. Characteristics of H. influenzae, an obligate human commensal found principally in the upper respiratory tract, meet the requirements of its relatively simple lifestyle with a small genome that lacks many regulatory circuits. H. influenzae is capable of generating distinct phenotypes that differ primarily in expression of surface proteins and LPS components due to microsatellites, which are rare in the Enterobactereacae but are common among other Gram-negative mucosal pathogens such as Neisseria and Helicobacter. Diseases common to this strain include systemic infections such as bacteraemia, meningitis, septic arthritis and pneumonia in young children, caused primarily by H. influenzae possessing the type b capsule (Hib). Respiratory infections such as pneumonia, otitis media, sinusitis, and bronchitis are caused primarily by non-encapsulated strains. Major virulence factors in Haemophilus include the adherence factors HMW1/HMW2, Haemagglutinating pili, Hap, Hia/Hsf, OapA, and P5 protein. The major endotoxin is LPS. Immune evasion is facilitated through the P2 protein.

Helicobacter are small curved Gram-negative bacteria closely related to Wolinella succinogenes, Campylobacter spp., and Acrobacter spp. Isolates are characterized biochemically by the presence of urease, catalase, and oxidase activities. Many Helicobacter species can colonize the intestines or biliary tracts of humans and other mammals, where they cause an inflammatory response. H. pylori are extraordinary bacteria in their ability to colonize the human gastric mucosa, an inherently inhospitable acidic environment, and to persist in this niche for many decades, despite the development of host immune and inflammatory response. Persistent colonization with has been recognized as a significant risk factor for serious gastroduodenal disease. H. pylori is a genetically diverse species, with strains differing markedly in their virulence. Major virulence factors found in Helicobacter include adherence factors BabA, HopZ, and SabA. The major endotoxins are LPS and VacA.

Legionella are non-spore-forming, Gram-negative bacilli, a member of the γ-proteobacteria, and so far, 42 species and 65 serogroups of Legionellae have been described. Intracellular replication within selected host cells is the primary and perhaps sole means of proliferation in the environment. Many of these species are reported to be pathogenic for humans, but L. pneumophila is the most frequently isolated species associated with disease. This species is a intracellular pathogen that invades and replicates within a protective phagosome inside alveolar macrophages. Two phase of growth are observed, a replicative phase—characterized by sodium resistance, and non-flagellated cells, having low cytotoxicity, and an infectious phase—characterized by organisms that are morphologically short, thick, flagellated and highly cytoxic. Killing and lysis of macrophages require two steps, the triggerance of apoptosis at the early stage and induction of pore formation later in the infection process. The key to L. pneumophila's virulence appears to be its ability to prevent phagosome-lysosome fusion. The major virulence factors in Legionella include Hsp60, a type IV pili. Endotoxins include LPS.

Listeria are Gram-positive, rod-shaped, non-capsulated, non-sportulating bacterium. The genus consists of six species: L. monocytogenes, L. seeligeri, L. welshimeri, L. innocua, L. ivanovii, and L. grayi. L. monocytogenes and L. ivanovii are typical facultative intracellular parasites, able to enter, survive and multiply inside phagocytic and nonphagocytic cells. The mode of entry is a zipper mechanism. This entails the zippering of the host cell membrane around the bacterium as it enters. Bacterial ligands interact with a surface molecule on the host cell. The receptor is generally a protein involved in cell adhesion and/or activation of the cytoskeleton machinery.

The ligand-receptor interaction induces local rearrangements in actin cytoskeleton and other signals that culminate in the tight envelopment of the bacterial body by the plasma membrane. Listeria spreads directly from cell to cell by actin-based intracellular movements. L. monocytogenes infects both human and animals causing meningitis, sepsis, and abortion. L. ivanovii is restricted to sheep and cattle, in which it causes septicemic disease, neonatal sepsis and abortion, but no brain infection. The infectious disease caused by these bacteria is known as listeriosis. The other species are generally considered nonpathogenic

Mycobacterium are a genus of bacteria in the family of Mycobacteriaceae. They are Gram-positive, the majority of the over 50 species are non-pathogenic environmental bacteria related closely to the soil bacteria Streptomyces and Actinomyces. A few species are highly successful pathogens, including Mycobacterium tuberculosis, M. leprae and M. ulcerans. Pathogenic mycobacteria are extraordinary adept at establishing long-term infections that can manifest as acute or chronic disease or be clinically asymptomatic with the potential to resurface later. M. tuberculosis is the causative agent of tuberculosis. M. leprae is the causative agent of leprosy. M. ulcerans is the causative agent of Buruli ulcers. Major virulence factors in Mycobacterium include Antigen 85, LAM and MmaA4. The major mycobacterial toxin is Phospholipase C.

Neisseria are Gram-negative diplococci with adjacent sides flattened. The genus contains two human pathogenic species, N. gonorrhoeae and N. meningitidis, as well as a number of other species that are either pathogenic to animals or are normal flora in either humans or animals. N. gonorrhoeae and N. meningitidis are exemplary for their ability to adapt to their sole host, the human. Both species possess the ability to colonize human mucosal tissues. N. gonorrhoeae primarily infects the uro- or anorectal mucosa following intimate sexual contact, while N. meningitidis colonizes the nasopharynx after the inhalation of infected respiratory droplets. One extraordinary characteristic of pathogenic neisseriae is their enormous capability to vary their surface structures. Mode of entry into host cells occurs through the zipper mechanism discussed above. N. gonorrhoeae is the etiological agent of gonorrhea, while N. meningitides is the etiological agent causing epidemic meningitis. Major virulence factors include LOS, Type IV pili.

Pseudomonas are ubiquitous bacteria that belong to the γ-Proteobacteria family. The Pseudomonas genus contains the clinically important human pathogen P. aeruginosa, the agriculturally important plant pathogen P. syringae, and the nonpathogenic bioremediation agent P. putida. P. aeruginosa is a major opportunistic human pathogen, notable for its ability to form biofilm and has the best-characterized quorum-sensing systems among Gram-negative bacteria. P. aeruginosa can cause a variety of opportunistic infections ranging from eye infections in contact lens wearers to burn and wound infections leading to septic shock, and lung infections in cystic fibrosis or other immunocompromised patients.

Salmonella are Gram-negative bacilli, comprising two species: S. enterica, which is subdivided into over 2,000 serovars, and S. bongori. Based on genetic similarity and host range, the species has been further divided into six subspecies: enterica (Group 1), salamae (Group 2), arizonae (Group 3a), diarizonae (Group 3b), houtenae (Group 4), and indica (Group 6). S. bongori was initially categorized as subspecies 5. S. enterica includes many of the serotypes pathogenic for humans, including S. typhi and S. typhimurium. Salmonella traverse the intestinal mucosa through M cells, colonize Peyer's patches, and spread via the lymphatics and bloodstream to the liver and spleen. In contrast to Shigella, Listeria and Richettsiae, which escape from their nascent membrane-bound compartment and replicate in the cytoplasm, Salmonella manages to survive within its membrane-bound vacuole. S. typhimurium is a leading cause of human gastroenteritis, and is used as in mouse models of human typhoid fever. S. typhi is a human-specific pathogen causing the systemic febrile illness typhoid fever

Shigella are Gram-negative enteric bacilli, closely related to Escherichia. They are divided into four species: S. dysenteriae, S. flexneri, S. boydii and S. sonnei. Shigella are facultative intracellular pathogens. The initial entry route in humans is M cells in the follicle-associated epithelium (FAE) that overlies the mucosa-associated lymph nodes. Entry into polarized epithelial cells occurs most efficiently from the basolateral side (Salmonella and EPEC/Shiga toxin producing E. coli are able to interact with host cells from the apical side). Shigella, Listeria and Rickettsia are the only three bacterial genera found so far that are able to escape from the phagocytic vacuole and to use cytoplasmic cytoskeletal components to achieve movement and lead to cell-to-cell spread. Diseases attributable to Shigella infections include shigellosis and dysentery caused by Shiga toxin (S. dysenteriae (serotype 1 only)).

Staphylococcus are Gram-positive spherical bacteria belong to Micrococcaceae family. They are classified into two major groups: aureus and non-aureus. S. aureus is one of the major causes of community-acquired and hospital-acquired infection. Of the non-aureus species, S. epidermis is the most clinically significant. Staphylococcus are primarily an extracellular pathogen. Adherence is mediated by surface protein adhesins called MSCRAMMs (microbial surface components recognizing adhesive matrix molecules). One feature that contributes to the virulence of S. epidermidis is the ability to adhere to plastic and to form a biofilm. S. aureus causes a wide variety of diseases, ranging form superficial abscesses and wound infections to deep and systemic infections such as osteomyelitis, endocarditis and septicaemia, toxic-shock syndrome, staphylococcal scarlet fever, and scalded skin syndrome. S. epidermidis is the etiological agent of catheter-associated infections, and endocarditis, typically resulting from biofilms on plastic implants. Toxins include α-hemolysin, β-hemolysin, δ-hemolysin, γ-hemolysin, Exfoliative toxin, PVL, SE, and TSST-1.

Streptococcus are a heterogeneous group of Gram-positive aerobic bacteria which appear as chains under microscopic observation. The genus is divided into three groups by the type of hemolysis on blood agar: β-hemolytic (clear, complete lysis of red cells), α hemolytic (incomplete, green hemolysis), and γ hemolytic (no hemolysis). Lancefield classification is based on antigenic differences in cell wall carbohydrates (groups A to V). Surface carbohydrate antigens of S. pneumonia do not correspond to a specific Lancefield group, it can be considered a pyogenic (pus-producing) strain of Streptococcus. S. pyogenes is responsible for a wide variety of diseases, including pharyngitis, scarlet fever, impetigo, erysipelas, cellulitis, septicemia, toxic shock syndrome, necrotizing fasciitis and the sequelae, rheumatic fever and acute glomerulonephritis. S. agalactiae is responsible for neonatal meningitis, bacterial sepsis and pneumonia. S. pneumoniae is the major etiological agent of pneumonia.

Treponema pallidum is the causative agent of syphilis. It is a spirochete, a helical to sinusoidal bacterium with outer and cytoplasmic membranes, a thin peptidoglycan layer, and periplasmic flagella. Mechanisms of T. pallidum pathogenesis are poorly understood. No known virulence factors have been identified, and the outer membrane is mostly lipid with a paucity of proteins. Consequently, prior art diagnostic tests for syphilus are suboptimal.

Vibrio are Gram-negative, part of the family Vibrionaceae, which is also includes the genera Aeromonas and Plesiomonas. V. cholerae is one of several medically important species, the others being V. parahaemolyticus and V. vulnificus. V. cholerae is well recognized and extensively studied as the causative agent of the human intestinal disease cholera. The disease occurs at the mucosal surface, with no invasion by the microbe into deeper tissue, and the disease symptoms are primarily due to the action of a single molecule, the cholera toxin.

Yersinia are Gram-negative coccobacillus, composed of 11 species, three of which are pathogenic for rodents and humans: Y. pestis, Y. pseudotuberculosis and some biotypes of Y. enterocolitica. Virulence is essentially conferred by the presence of a conserved 70 kb plasmid, called pYV for plasmid involved in Yersinia virulence. Yersinia displays a trophism for lymphoid tissue and a remarkable ability to resist the primary immune response of the host due to the Ysc-Yop type III secretion system. Mode of cellular entry occurs by the zipper mechanism. Y. pestis is the etiological agent of bubonic plague, or black death. Y. pseudotuberculosis is an agent of mesenteric adenitis and septicaemia. Y. enterocolitica, the most prevalent in humans, causes gastrointestinal syndromes, ranging from acute enteritis to mesenteric lymphadenitis.

### Pathogen Reference Library

The present invention provides for the creation of a Proteomic Pathogen Reference Library (PPRL) that is electronically searchable, that is used in conjunction with a cell-based assay using APC for detecting biological pathogens and chemical toxins, in humans for diagnostic and therapeutic purposes, and detection of same in the environment, and in livestock (such as BSE and bacterial contamination). The Proteomic Pathogen Library is also useful as a reference for screening food(s) for bacterial pathogens that could be potentially used by terrorists to compromise the integrity of the food supply, and to generally detect pathogenic contamination arising from more benign but equally dangerous sources.

While pathogen signatures are obtainable, more preferably gene and proteomic changes in an APC, preferably the totality of the cellular gene and proteomic changes in the APC following pathogen contact, are assayed to provide specific detail as to the pathogen contacted. In one aspect, a pathogen reference library is constructed from individual electronically searchable records or data sets of various APC contacted with pathogenic bacteria. This is further detailed in Example Six. The pathogen reference library facilitates diagnosis where a sample obtained from a subject having an infection from one or more unknown pathogens is compared to various data sets in the pathogen reference library until an approximate match for the APC signature profiles in the subject sample is detected in the pathogen reference library records.

To construct a pathogen reference library, first individual signatures for known pathogens are obtained as described, for example by spectrographic methods. These are used as reference signatures, and multiple signatures from each reference sample are used to derive a consensus signature for the individual pathogen or agent. The consensus signatures are used to create a knowledge base using which unknown pathogenic samples can be identified based on the information stored in the pathogen database. A proteomic pathogen reference library is preferred (PPRL), having records of APC proteomic changes in response to antigen contact. In certain embodiments, the library includes data records having the consensus signatures of APC that have been contacted with a particular pathogen, together with other data that characterize the pathogen. The other data include ASCII data (for example, including simple alphanumeric data) representative of data fields pertaining to the pathogen sample, such as, for example, date obtained, strain information, buffer composition, source of the sample, APC cell type, etc.

The data records are stored in any commercially available standard database, or a customized database may be developed to store the data records such that the data records may be indexed and easily accessed by various computing applications. The data records may also be stored as XML files or another suitable tagged formats so that the data files can be easily transmitted from one computing system to another.

In one embodiment, the signatures are stored in a database, for example in the form of electronic records in a computer. Such a database may be integrated with or provided by a knowledge base, as discussed earlier herein. The database may be networked, for example, by a packet switched network. Access to the database can be effectuated by a number of connectivity protocols, such as a TCP/IP connection or any suitable Internet connection. The database may also be standalone, e.g., contained on optical media such as a compact disc. A computer-implemented database is typically a collection of data, organized in the form of tables or other user definable fields, as is well known to those skilled in the art. In certain embodiments, the present invention provides for storing the signatures in the assigned database table in the relational database, and the database can be searched to return a search result including data identifying a set of element objects satisfying search terms specified for one or more searchable fields. Search results can be stored as lists of element objects that satisfy the search terms of a query. Element object values can be displayed for one or more displayable fields. In certain embodiments, a laboratory data management system may be suitably used as the database to store elements of the data model that are required for implementing the signature and feature value database and for searching the database for finding matches corresponding to signatures of unknown pathogens. Searches by MS peak, name, structure, substructure, and property fields, such as technique, molecular weight, CAS Registry Number, and chemical synonyms etc. are permitted. Communication between client processes and database server process may be implemented using techniques that are well known to those skilled in the art. For example, such communication may be provided through the use of a persistent representation of data in a self-describing extensible format such as XML. Client processes receive or generate data derived from an experiment and package that data using known techniques in a format (e.g., an XML data stream) for communication to database server process. Upon receiving such a communication from a client process, the database server process parses the incoming data stream to extract object descriptions from the data stream.

In addition to storing the data records in a standard or custom database, record storage and management may be enhanced by the use of a suitable knowledge management tool. Such knowledge management tools typically allow a user to identify the information they want to track, organize it in a coherent manner, and add information and reference files to enhance knowledge about a record. For example, for a particular pathogenic sample, the knowledge management tool may allow the coherent storage of information related to that pathogenic sample, including the APC used to obtain the particular signature, the organisms characteristics, related sample data, and other information or comments added by the library user. The knowledge base thus provides a collection of individual reference data records that can be searched by a search application in order to identify or develop a subset of the data records that best meet specific inclusion or exclusion criteria provided by the search application.

An exemplary informatics reference system is the Know-It All® Informatics System from BioRad Laboratories, designed to work seamlessly with numerous spectral databases.

The following example details using mass spectroscopy techniques to create a proteomic pathogen reference library (PPRL). First, the database is built, comprising a plurality of data records, each record having spectral information about a particular pathogen and a specific APC. Known pathogenic samples are contacted with APCs, which are then processed through a mass spectrometer to generate signatures of these antigen contacted APCs. The spectral signatures are stored, along with molecular or organismal structure information, instrument parameters, physical and chemical properties of the pathogen sample, images and documents and other information, and compiled into a data set, which is archived as a single data record. The reference data records are then organized, processed, and annotated by a knowledge management tool before being stored in the database. Additional databases of gene and protein (biomarker) expression information are desirable, particularly where they have records that can be parsed along with the spectral database. Hyperlinking of information allows data to be stored on distant servers, but allows it to be accessed from one or more remote locations.

To characterize an unidentified pathogen, a spectrometric analysis would be performed on APC's contacted with an unidentified pathogen to produce a spectrographic signature file and the associated data. A preferred spectrographic signature is a mass spectrometry signature. The search application or expert system would then perform a comparison of the unidentified pathogen against the database of known pathogens to make the desired identification, which is based on record similarities and user-defined rule based approaches. For MS spectral searches, peak location uncertainty must be accounted for when comparing spectra. Preferably, peak location uncertainty tolerances are set by the user of the system. In addition, since the search algorithm is comparing peaks from unknown and reference spectra, a user should be permitted to specify positions for which the searched spectra should or shouldn't have a peak, specify the minimum size and numbers of peaks compared, and include or exclude considerations based on peak intensities. Based on these criteria, the system identifies potential reference records that are similar to the unknown queried record.

The identified record suggests exposure to a particular pathogen, and would then be reported back to the user in a suitable reporting format. In the event no match is found between the unknown record and those in the reference database, then potentially a new record has been found that could be analyzed and added to the database as described. Even if the unidentified pathogen name cannot be determined, it's signature can be added into the database for future matches against other unidentified pathogens to establish the repeated occurrence of this unknown pathogen. In certain embodiments, the unidentified pathogen may be compared against a local database of known pathogens and if no match is found, the unidentified pathogen may be compared against other networked databases of known pathogens that may be located at a remote location. In this manner, even pathogens that are relatively rare is a local area may be matched to a known pathogen in a remote database in an area where that pathogen may be more prevalent, or more widely studied. For example, local hospitals may attempt to match spectroscopic signatures of unidentified pathogens obtained from patient subjects against those pathogen records contained in a database at the Center for Disease Control, in Atlanta.

In general, the search application or expert system identifies input test samples by comparing their spectroscopic signatures to the signature records of known pathogens stored in the database. Preferably the signatures are of APC obtained from a subject having an unknown infection, compared to APC signatures of those that have been contacted with reference pathogens. The expert system typically includes a user interface to provide information to and receive instruction from the user of the PPRL. In certain embodiments, a user interface allows the user to operate the system over a network such as a packet switched network, although standalone systems may also be used. The search application or expert system provides a computer readable instruction set and configuration data that identifies and selects specific records in the PPRL database.

The expert system or search algorithm typically includes a combination of decision trees, analysis logic, and pattern recognition routines to select records meeting specific criteria. The analysis logic routines allow the system to interact with the user through the user interface, retrieves known pathogenic signatures from the knowledge base for comparison against the identified sample, and prepares output documentation in an appropriate output format.

The decision trees routines may be used in narrowing the universe of records in the knowledge base that need to be parsed. For example, if the user knows the test sample contains a bacterial pathogen, the user can input this information through the interface, and the expert system will exclude chemical agent signatures and viral signatures from consideration. Pattern recognition routines parse the narrowed subset of records and identify the best match. The pattern recognition software is preferably based on neural net technology and uses pattern recognition in identifying the unknown pathogens based on their spectrographic signatures, as discussed in more detail further herein.

In one embodiment, a set of feature values are extracted for each consensus spectrographic signature of a known pathogen (or APC contacted with the same). These feature values can then be stored in a database either as a set of the feature values or as a composite value derived from the set of feature values. When an unknown pathogen is being evaluated, its set of feature values is extracted and compared against the database to find an exact or approximate match. For example, if sets of feature values of known pathogens are stored in the database, a set of feature values for the unknown pathogen is derived and compared to the database to find whether it matches an existing set of feature values in the database. If a match is found, the unknown pathogen is identified as the known pathogen whose set of feature values in the database were matched. Likewise, if a composite value derived from the set of feature values is stored in the database, a composite value is also derived for the unknown pathogen and the composite value is then compared to find a match in the database. Once a match is found, the unknown pathogen is identified as the known pathogen whose composite value in the database is matched. As would be recognized by those skilled in the art, the set of feature values for the spectrographic signatures could simply be representative of the set of relative intensities in the spectrographic output. Alternatively, a composite value could be derived from the set of relative intensities in the spectrographic output.

### Pattern Recognition

In certain other embodiments, well known techniques used in pattern recognition are used to identify the spectrographic signatures from unknown pathogenic samples based on a knowledge base of data and/or images derived from the spectrographic signatures of known pathogenic samples. The use of neural networks in solving such pattern recognition problems is discussed extensively in the literature. See, e.g., CORNELIUS T. LEONDES ED., IMAGE PROCESSING AND PATTERN RECOGNITION (Academic Press 1998).

One definition of pattern recognition is an information reduction process in which visual or logical patterns are assigned to classes based on the features in the patterns and their relationships. Therefore, this definition comports well to problem at hand in which the spectrographic signatures (including data derived therefrom) is assigned to one of the known classes which correspond to the spectrographic signatures (including data derived therefrom) of known pathogens.

One generic model of the pattern recognition solution is described in Lampinen et al., Pattern Recognition, in IMAGE PROCESSING AND PATTERN RECOGNITION, 1-53 (Cornelius T. Leondes, ed., Academic Press 1998), the contents of which are incorporated herein in its entirety. The model describes the following states in a pattern recognition solution: (1) Data Collection; (2) Registration; (3) Preprocessing; (4) Segmentation; (5) Normalization; (6) Feature Extraction; (7) Classification; and (8) Postprocessing. Each of these stages are discussed in the following paragraphs.

### (1) Data Collection

The data collection stage includes the collection of the spectrographic signatures for the known pathogens (the training data) as well as the collection of the spectrographic signatures for the unknown pathogens that are to be identified or classified. The process of collection of the spectrographic signatures has been discussed in greater detail in other parts of this application.

### (2) Registration

In the registration stage, typically very rudimentary fitting of the data to the model is performed. For example, in the speech recognition context for pattern recognition, the registration stage may include determining the parts of the speech which are pure noise so that the utterances that need to be classified can be isolated. In context of the spectrographic signatures, this stage is straightforward since the training data (signatures of known pathogens) and the test data (signatures of the unknown pathogens) will be fairly easily identified based on the output of the spectrographic system.

### (3) PreProcessing

Preprocessing of data is performed to reduce the effect of noise on a signal. Therefore, features of the data that hinder the pattern recognition process may be removed while features of the data that promote the pattern recognition problem may be enhanced.

### (4) Segmentation

In this stage, the preprocessed data is split into subparts that are meaningful entities for classification. In the context of identifying spectrographic signatures, the task of identifying the signatures (based on the relevant outputs from the spectrometers) is relatively straightforward.

### (5) Normalization

One of the problems in all pattern recognition problems is variation in the signatures to be identified either based on the inherent variation of the pathogens to be identified or based on variation introduced by the data collection and processing stages. Therefore, one of the approaches that may be used is to use feature extraction or classification algorithms that are invariant to the variations of the objects being classified. This process is called normalization and this process has the side effect of at least some loss of degrees of freedom. This results in a dimension reduction in the intrinsic dimensionality of data. In the context of the spectrographic signatures, normalization can easily be implemented based on the pattern of the relative intensities of the spectrometer output. Therefore, rather than using absolute values of any kind, the use of the relative intensities (or signatures) serves to normalize the data for further use in pattern recognition.

### (6) Feature Extraction

The purpose of feature extraction is to extract from the raw data the information that is most relevant for classification purposes, in the sense that it serves to minimize the variation within a class while maximizing the variation between classes. While feature extraction also serves to reduce the dimensionality of data, it serves to avoid the problem of the “curse of dimensionality,” in which increasing the dimensionality of the features space rapidly results in a sparseness of training data which causes a decrease in the classification performance. Neural networks may be used in the process of feature extraction as discussed, for example, in Lampinen et al. at pages 11-20, which is incorporated herein in its entirety.

### (7) Classification

This is the most important stage in which unknown pathogenic signature is classified based on the known pathogenic signatures stored in the knowledge base. The output of the classification process would be a discrete selection of one of the classes corresponding to one of the known pathogens or an indication that none of the known pathogenic classes was matched. In addition, the classification process may also indicate a probabilistic measure which indicates a level of confidence at which a certain classification was made.

Statistical and neural classification method are discussed in Lempinen et al. at pages 20-37, the disclosure of which is incorporated herein in its entirety. The statistical methods include parametric methods in which a specific functional form is assumed for the feature density vectors while non-parametric methods refer directly to the available exemplary data. In the context of the matching reference signatures, both parametric and non-parametric method may be advantageously used.

Neural methods are often classified based on their learning process: supervised learning algorithms require that all exemplary data be classified before the training phase begins, while unsupervised algorithms may use unlabeled data as well. For the classification of the unknown pathogenic signatures, the supervised learning algorithms appear advantageous whereas the unsupervised learning algorithms may be more suitable for the feature extraction process, for example.

In certain embodiments, tree-based classifier models may be used. For example, if a classification tree is a binary tree where at each node a decision is made whether to branch to the left or right based on a criteria, for example, by comparison to a feature value. The tree growing algorithm recursively splits the pattern space into hyperrectangles while trying to form maximally pure nodes. Stopping criteria may be used to keep the tree reasonably sized. A commercially available tree based classifier is provided by the S-Plus statistical software package which is described, for example, in W. N. VENABLES AND B. D. RIPLEY, MODERN APPLIED STATISTICS WITH S-PLUS (Springer Verlag, New York, 1994).

Prototype classifiers may also be advantageously used in certain embodiments. They keep training samples in memory (or load them from a knowledge base) and classify based on the distance between the memorized training samples and the input data. Some examples of such classifiers include the k-nearest neighbor classifier (k-NN), the learning vector quantizer (LVQ) algorithm, and the learning k-NN classifier which are all described in Lempinen et al. at pages 30-32 which are incorporated herein by reference.

### EXAMPLE ONE

The following example details the use of APC's as biosensors for disease. Reference standards of DC exposed to various pathogens are created, which are used in subsequent patient assays to determine exposure and to qualitate the immunological response to the pathogen.

Gene expression analysis: DC and Macrophages/monocytes from various donors are cultured in the presence and absence of pathogens, to initiate a response to the pathogen, then harvested. Total RNA is extracted from uninfected and infected cells, and used to create a reference array. Alternatively, the total RNA is converted to cDNA, before being fabricated into the reference array. Patient derived samples of DC are recovered, the nucleic acids extracted, and hybridized to the microarray then scanned. In the microarray data processing we use a filtering approach based on a fix change in average difference intensity values. For analysis of the raw data we use GenNet, an extremely robust expression data analysis platform. Using such a platform meets high-throughput demands, and is scalable.

Pathogen Genotyping: Since DC and Macrophages serve as pathogen reservoirs, enrichment of these cells and the use of genotypic analysis for the presence of the pathogen provides a novel screening method. Conventional methods of viral and bacterial disease diagnosis require the detection of the pathogens themselves, e.g., by blood cultures, or detection of pathogen-specific proteins or DNA/RNA, e.g., by PCR, and correlation of these findings with clinical symptoms. To overcome the limitations associated with conventional screening methods, we have developed high throughput genotyping assays, which are used to screen large numbers of pathogens. In addition, these assays are capable of detecting molecular variation in microbial strains; thus allowing distinction of, for example, route of transmission, origin, and relationship of a particular bacterial, viral, fungal, or parasitic strain, etc.

Mass Spectrometrv (MS): To improve the success and productivity of peptide identification we have used the SELDI and MALDI time of flight (TOF) mass spectrometers. These are the most commonly used mass spectrometry methods for detecting peptide mass fingerprinting. This MALDI-O-TOF system uses orthogonal injection to introduce sample ions from the MALDI sources into a reflection TOF mass spectrometer. The MALDI sources of a conventional axial MALDI-TOF systems (linear or reflection mode) and is directly linked to the TOFMS. This direct linkage affects the instruments accuracy, resolution, and sensitivity because any discrepancies associated with the sample target are transferred to the detector. In contrast, using orthogonal geometry, the MALDI source is separated from the TOF, thus, eliminating discrepancies, increasing performance and simplifying method development. The protein signatures found in untreated cultured compare to treated cultures are established.

Bio-marker detection: To detect bio-markers, we use commercially available pattern recognition and discover software from Eclipse Diagnostics, or similar software. This software allows for the rapid detection of genomic and proteonomic bio-markers and other complex biological relationships. These biomarkers are part of the database and are used as a pathogen-specific reference.

Advantages First, myeloid cells concentrate pathogens within the cell; thus, improved sensitivity of detection. Second, cDNA microarray technology is a high resolution technology capable of analyzing 52,000 or greater genes per sample and is independent of culturing the pathogen from blood. Our method of microarray construction involves a longer 70mer probe design and 30-fold internal redundancy per gene. Third, the ability to diagnose before symptoms occurs. In the case of bioengineered pathogens, traditional microbiology, ELISA or PCR based technology has to be created to detect new the new pathogen. By evaluating key cellular pathways (e.g. apoptotic, inflammatory, NFkB, and inflammatory mediators), we can detect early events in exposure. We have developed a method of rapid enrichment (1.5 h) of DC and Macrophages from the blood, which does not require prolonged culturing, or use of costly cytokines to force differentiation. In contrast, contemporary methods require culturing DC precursors for 7 days. DC in culture with prolonged exposure to cytokines is known to induce specific alteration in cellular pathways, have been demonstrated to modify pathogen infectivity, gene activation, and protein synthesis. High-throughput (HT) gene arrays allow the analysis of greater than 1,000 samples in per day. The number samples processed per day is equipment dependent which is also the case for the proteonomic technology (e.g. 10,000 samples/day). The integration of the HT system with the genomic and proteonomic database improves detection efficiency and also allows the real-time monitoring of progression of disease. Collectively, the combination serves to provide highly complex genomic- and proteonomic-based arrays and information databases for hospitals and laboratories, that can be shared in real time over networks.

The present invention provides for methods of rapidly identifying pathogens in the body and in the environment. The following pathogens are amenable to detection and characterization using APC and the techniques described herein: bacteria, bacterial toxins, viruses, fungi, prions and protozoa.

Representative bacteria that are presented to APC to create pathogen exposed APC signatures include, Gram positive and Gram negative bacteria such as Staphylococcus, such as S. epidermis and S. aureus; Micrococcus; Streptococcus, such as S. pyogenes, S. equis, S. zooepidemicus, S. equisimilis, S. pneumoniae and S. agalactiae; Corynebacterium, such as C. pyogenes and C. pseudotuberculosis; Erysipelothrix such as E. rhusiopathiae; Listeria, such as L. monocytogenes; Bacillus, such as B. anthracis; Clostridium, such as C. perfringens; and Mycobacterium, such as M. tuberculosis and M. leprae. Gram negative bacterial species are exemplified by, but not limited to genera including: Escherichia, such as E. coli 0157:H7; Salmonella, such as S. typhi and S. gallinarum; Shigella, such as S. dysenteriae; Vibrio, such as V. cholerae; Yersinia, such as Y. pestis and Y. enterocolitica; Proteus, such as P. mirabilis; Bordetella, such as B. bronchiseptica; Pseudomonas, such as P. aeruginosa; Klebsiella, such as K. pneumoniae; Pasteurella, such as P. multocida; Moraxella, such as M. bovis; Serratia, such as S. marcescens; Hemophilus, such as H. influenza; and Campylobacter species. Other species suitable for assays of the present invention include spirochetes such as those causing Lyme Disease, Enterococcus, Neisseria, Mycoplasma, Chlamidia, Francisella, Pasteurella, Brucella, and Enterobacteriaceae. Also detectable are CDC biological pathogens A, B, and C biological pathogens. Further examples of pathogenic bacterial species that are detectable according to the invention are obtained by reference to standard taxonomic and descriptive works such as Bergey's Manual of Determinative Bacteriology, 9th Ed., 1994, Williams and Wilkins, Baltimore, Md.

Representative viruses that are presented to APC to create pathogen exposed APC signatures include, adenovirus (such as can be found in infantile gastroenteritis, acute hemorrhagic cystitis, non-bacterial pneumonia, and viral conjunctivitis), herpesvirus (such as herpes simplex type I and type II, varicella zoster (the etiological agent of chicken pox), cytomegalovirus, and mononucleosis (the etiological agent Epstein-Barr virus)), poxvirus (the etiological agent for such disorders as smallpox (variola major and variola minor), Hepatitis A, B, and C, vaccinia virus, hantavirus and molluscum contagiosum), picornavirus (such as rhinovirus (the common cold, also caused by coronavirus)) poliovirus (poliomyelitus)), an orthomyxovirus or paramyxovirus (such as influenza, and respiratory syncytial virus (RS)), parainfluenza virus (including such diseases as mumps), and rubeola (measles), rhabdovirus (rabies), vesicular stomatitis (VSV), togavirus such as rubella—the etiological agent causing German measles, and togaviridae causing encephalitis (EEE, WEE, and VEE), flavivirus such as the etiological agent causing Dengue Fever, West Nile Fever, Yellow Fever, and encephalitis, bunyavirus and arenavirus, reovirus, coronavirus such as the agent causing SARS, hepatitis, a papovavirus infection such as papilloma virus, a retroviral infection such as HIV, HTLV-I, and HTLV-II.

Representative fungi that are presented to APC to create pathogen exposed APC signatures include for example, Candida, such as C. albicans; Cryptococcus, such as C. neoformans; Malassezia (Pityrosporum); Histoplasma, such as H. capsulatum; Coccidioides, such as C. immitis; Hyphomyces, such as H. destruens; Blastomyces, such as B. dermatiditis; Aspergillus, such as A. fumigatus; Penicillium, such as P. manieffei; Pseudallescheria; Fusarium; Paecilomyces; Mucor/Rhizopus; and Pneumocystis, such as P. carinii. Subcutaneous fungi, such as species of Rhinosporidium and Sporothrix, and dermatophytes, such as Microsporum and Trichophyton species, are amenable to prevention and treatment by embodiments of the invention herein. Other disease causing fungi that can be detected include Trichophyton, Microsporum; Epidermophyton; Basidiobolus; Conidiobolus; Rhizopus Cunninghamelia; Rhizomucor; Paracoccidioides; Pseudallescheria; Rhinosporidium; and Sporothrix.

Representative protozoa that are presented to APC to create pathogen exposed APC signatures include the one or more single-celled, usually microscopic, eukaryotic organisms, such as amoebas, ciliates, flagellates, and sporozoans, for example, Plasmodium, Trypanosoma or Cryptosporidium.

The genes, peptides and proteins derived from the immune surveillance cells (APC) that are exposed to antigens, may also be used as part of a reference library based on their spectrographic signature, or may be used directly, for example to generate antibodies for use in FRET, flow cytometry and other multiplex-based detection methods. Preferred protein multiplex detection systems include those of Bender Systems, and BD Biosciences (such as the CBA system). Initial detection and screening of potentially exposed subjects can be done through e.g., spectrographic analysis. Anti-APC antibodies (to peptides upregulated after antigen exposure) are used in traditional flow cytometry and multiplex assays to follow the course of the disease.

### EXAMPLE TWO

The generation of antigen presenting cell (APC)-specific signatures is a two step process. The first step involves obtaining a population of immune cells, and the fractionation of cell membranes and the enrichment of proteins from the membrane, cytoplasm, and nucleus. Preferred cells are of the myeloid lineage, but PBMC's are suitable. Methods of enriching for myeloid cell populations, including DC, is described in U.S. Pat. Nos. 6,589,526 and 6,194,204. Myeloid cells include monocytes and dendritic cells, in roughly 90% to 10% proportions. Antigenic markers for monocytes include CD14+, HLA-DR or MHC class II, CD80+CD86+. Antigenic markers for DC include CD2+, CD5+, CD14+CD83+ and CD90+. These are obtained by positive or negative selection methods. Preferred cell types are myeloid, which express antigenic markers consistent with both DC and monocyte cells. It is currently preferred to use freshly isolated, i.e., blood purified myeloid cells instead of cultured myeloid cells.

The following is a suggested procedure for isolation of monocytes from PBMC: Buffy coats were isolated from healthy volunteers (Transfusion Therapy, Children's Hospital, Boston, Mass.) and washed and concentrated with PBS. The buffy concentrate was then incubated with a modified monocyte enrichment means, such as the RosetteSep Kit, commercially available from StemCell Technologies, for example. This rosette cocktail contains anti-CD3, anti-CD19, anti-CD54, and anti-CD62 monoclonal antibodies, which bind to T cells, B cells, NK cells and granulocytes. After 30 minute of incubation, this population was layered over ficoll gradient and centrifuged (Sorvall RT 6000, DuPont, Wilmington, Del.) at 2500 rpm for 30 min to separate the low density DC and Mo from the high density (T, B, granulocytes and NK cells) density fractions. The low density cell population was >95% CD14^{high} by flow cytometry. These cells were incubated with a 1:100 dilution of mouse mAb (in ascitic fluid) to human CD2 (1Old2-4C1 (anti-T112); Dana-Farber Cancer Institute, Boston, Mass.) (26) for 30 min at 4° C., washed, and incubated with goat anti-mouse IgG magnetic beads (Miltenyi Biotech). Following incubation, the preparation was passed through a magnetic column according to the manufacturer's instructions. The magnetic column retained the CD2+ cells, which were >96% pure, while the CD2-cells were >95% pure by flow cytometry with anti-CD2 and anti-CD14. A blocking buffer containing 10% v/v heat-inactivated pooled human serum (PHS) (Nabi, Boca Raton, Fla.) and human IgG (50 mg/ml; Immuno AG, Vienna, Austria) in HBSS without magnesium and calcium (Cellgro; Fisher Scientific, Pittsburgh, Pa.) was used to prevent nonspecific mAb binding during each stage of isolation or flow cytometric analysis. For morphologic and functional studies of freshly isolated, noncytokine-incubated CD2+ and CD2-Mo, we used culture medium (CM) containing RPMI 1640 (Cellgro) supplemented with 10% heat-inactivated PHS, 20 μg/ml gentamicin, 100 U/ml penicillin, and 100 μg/ml streptomycin (Life Technologies, Gaithersburg, Md.).

The second step involves obtaining mass spectra from the DC, for example using QSTAR (ABI), SELDI TOF from Ciphergen or proTOF from Perkin Elmer. Spectra are taken for naïve APC's and those exposed to pathogens, tumors, or other antigens. In this example, we describe the process for obtaining individual data sets (signatures) from pathogen-APC or pathogen-food samples (i.e. Listeria-APC, Listeria-milk) to create a profile for bacterially contaminated and uncontaminated milk. The signature of a Listeria infected individual, obtained from sampling of APC's from that individual, is also provided.

Each profile includes a proteomic signature of, for example but not limited to, the cell membrane, cytoplasmic proteins, and nuclear protein characteristics, protein charges (i.e. positive and negative), Cu2+ chelating properties, cleavage patterns of native or denatured proteins with various endopeptidase, and the like, of the agents under study, measured by such properties as m/z size (kD), m/z intensity (uAmps), and standard deviation quantities. The frequency of occurrence of identifying features in a signature is corroborated by obtaining spectra of replicate samples, preferably 3-4 samples, thereby providing a consensus signature.

Several commonly used methods for isolating, fractionating or enriching sample proteins are as follows, others are known in the art. PARTS Kit—this is a kit commercial available from Ambion, and allows for the rapid isolation of RNA and proteins from samples. This kit may be used for those studies involving the isolation of APC proteins from APC-viruses or bacteria cocultures. For more sensitive detection and characterization of samples, it is advantageous to use cellular membrane fractions, which are obtained by common molecular biology techniques. The combination of the membrane, cytoplasm, and nuclear proteins enhances the sensitivity of APC-based detection methods.

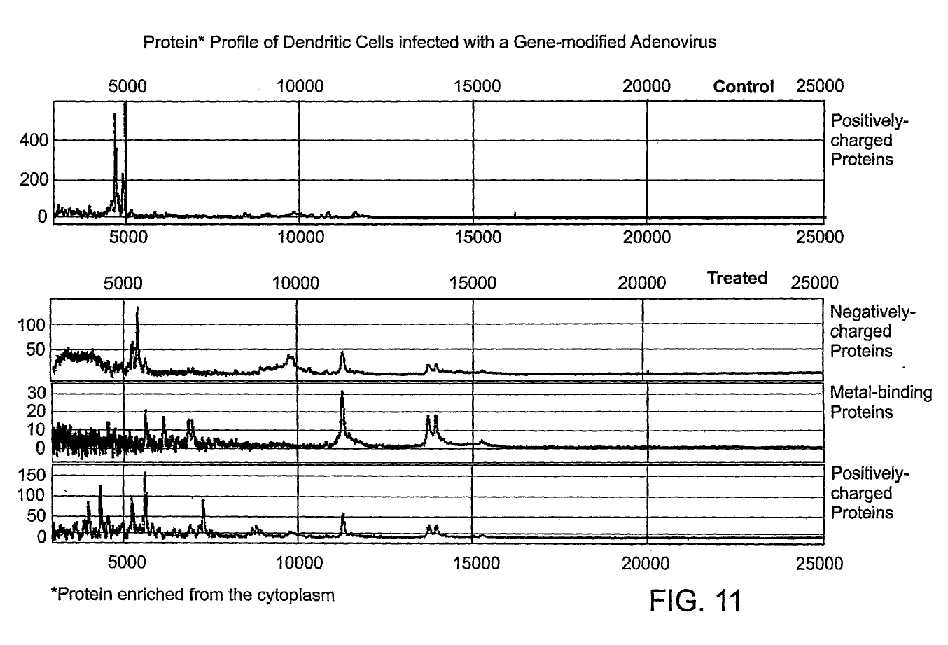

Bacterial lysis by sonication can be used for both Gram-negative and Gram-positive bacteria and uses sonication and optionally uses detergents, such as Tween, Triton X-100, digitonin, CHAPS, SDS, Nonidet and others, which are highly recommended for profiles of Gram-negative bacteria or microorganisms with a thick or tough cell membrane. This protocol was used in the milk studies shown in FIG. 11-13. Harvest bacteria from agar plate into 5 ml of TEN Buffer (10 mM Tris-HCl pH 7.4, 1 mM EDTA, 100 mM NaCl). Pellet bacteria at 5,000×g for 10 minutes. Subject the sample to 3 rounds of freeze/thawing in a dry ice/ethanol bath, thawing at 37° C. Resuspend the bacterial pellet into a small volume (0.1-0.5 ml) of ice-cold MTBS buffer (16 mM Na2HPO4, 4 mM NaH2PO4, 150 mM NaCl, 1% Triton X-100, 1 mM PMSF). Sonicate the suspension with 15 seconds burst followed by 30 seconds incubation on ice (4 rounds of sonication). Pellet bacterial debris at 14,000 rpm. Remove supernatant to fresh tube. Measure protein concentration by Bradford protein assay, standard curve, UV absorbtion or other method. Store at −70° C. until ready for SELDI analysis.

Repeat freeze thawing involves repeated freeze thaw cycles to shear the cells. Sample were frozen in a dry ice-ethanol mixture and thawed at 37 degrees C. These steps were repeated 4-times and samples were spun in a microfuge at 14,000 rpm for 5 minutes producing a clear supernatant. The supernatant was removed and stored at −70 degrees C. prior to sample runs. Bacterial lysis by French Press can be used for Gram-negative bacteria and is not recommended for Gram-positive bacteria. To perform this technique, resuspend pellets of bacteria in 20 mM HEPES pH 7.4, 50 mM NaCl, 1% Triton-X 100, 1 mM PMSF. Disrupt using a French Press at 750 psi. Remove cell debris by centrifugation at 20,000×g for 20 min at 4° C. Measure protein concentration in the supernatant by Bradford protein assay or similar assay. Store in aliquots at −70° C. until performing the SELDI technique. BugBuster Extraction kit—this is a commercially available kit sold by Novagen, which allows for gentle disruption of the cell wall of E. coli to release active proteins. This is a simple, rapid, low-cost alternative to French Press or sonication for releasing expressed target protein in a cell preparation. Alternatively, a buffer such as 10 mM Tris-HCl pH 7.4, 8 M Urea, 2% (w/v) CHAPS, 1 mM PMSF can also be used as lysis buffer. Store in aliquots at −70° C. until performing the SELDI technique.

The SELDI experimental protocol described below uses the IMAC ProteinChip Array (PCA). The IMAC Arrays are coated with an NTA functional group to entrap transitional metals for subsequent metal affinity binding proteins. In these profiling studies, arrays are charged with copper prior to applying sample to the surface. Selectivity is determined by concentration of imidazole in the binding buffer. Increasing concentrations of imidazole in the binding/washing buffer, reduces the binding of protein with weaker affinities for metal, thereby reducing background signals. The protocol for IMAC PCA is described in detail below and is similar to the other Ciphergen PCA protocols.

Using the 8 spot arrays: Assemble the PCAs in the bioprocessor and add 50 microliters of IMAC charging solution to each well. Vortex for 5 minutes at RT. Remove the buffer from the wells. Rinse with water. Add 50 microliters of IMAC neutralization buffer to each well. Vortex for 5 minutes RT. Remove the buffer from well and rinse. Add 150 microliters of the IMAC binding buffer to each well and vortex at RT for 5 minutes. Remove buffer. Repeat binding buffer wash steps twice. Next add 90 microliters of IMAC binding buffer and 10 microliters of sample and vortex for 30 minutes at RT. The ratio of IMAC binding buffer to sample concentration can vary depending of the desired protein concentration. Remove the sample and wash with IMAC binding buffer three-times, each wash requires a 5 minute agitation step. Once completed, rinse with de-ionized water, drain wells of the bioprocessors, and let air dry. Apply 1.0 microliters EAM (matrix) solution to each spot and let air dry. PCA are analyzed on the Ciphergen Chip reader.

By contrast, the Perkin-Elmer proTOF experimental protocol applies the isolated-protein sample directly to the MALDI surface. However, in this process the MALDI surface binds to everything in the sample (i.e. proteins and nonproteins). The manufacturer suggests cleaning up the samples, for example microscale protein purification using Millipore ZIP TIPS®, filtered ion exchange pipette tips capable of removing certain proteins, thereby reducing total protein levels and reducing the complexity of the sample mixture. However, this technique is sensitive enough such that the tip performance, variable from tip to tip, can impact the resultant signature.

The next step involves derivation of marker proteins from the APC interaction with the pathogen. Once the marker proteins have been isolated, the proteins provide templates for the generation of diagnostic antibodies. These antibodies to the derived APC proteins can be used for immunological assays, e.g., attached to a multiplexer or fluorescent readers for diagnostic purposes.

### EXAMPLE THREE

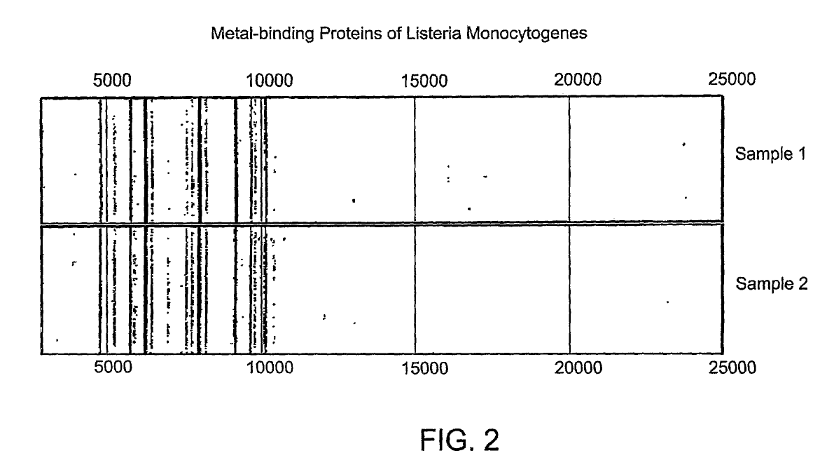

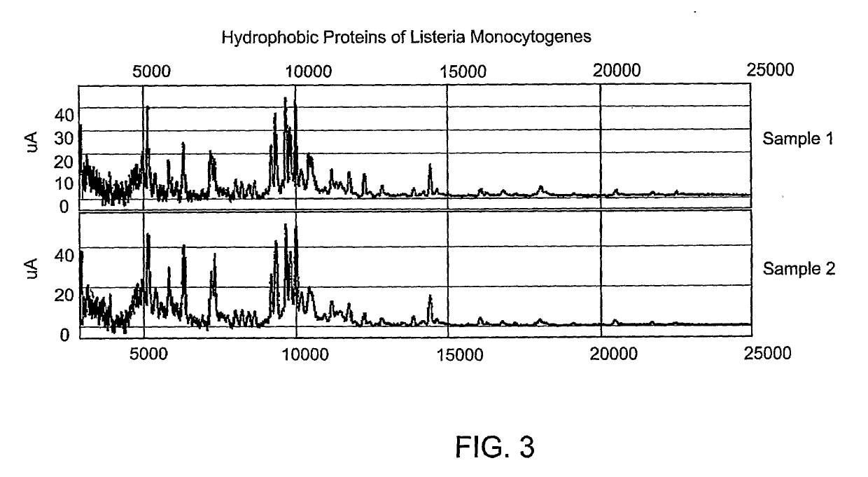

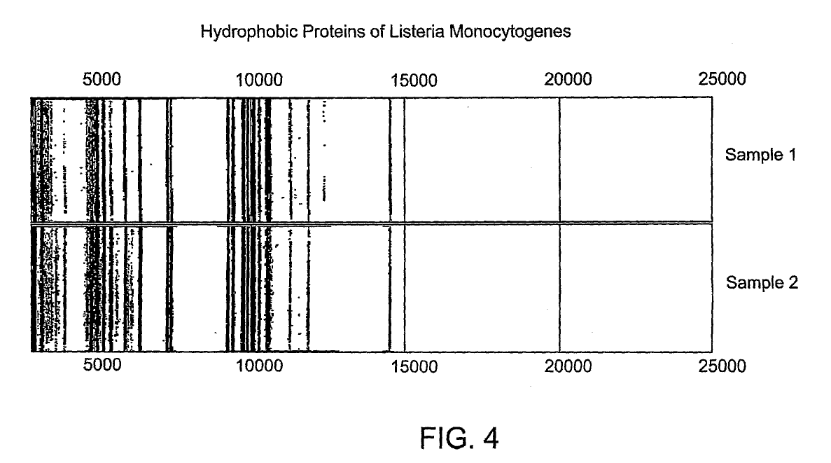

This experiment investigated the reproducibility of the APC derived signatures for DC exposed to bacterial and viral pathogens. Listeria monocytogenes was processed by the bacterial lysis and sonication methods described above and evaluated on metal-binding (FIG. 1-2) or hydrophobic surfaces (FIG. 3-4). Samples were processed in parallel and analyzed on different PCA chips on different chip analyzers. The results demonstrate excellent experimental reproducibility. Data is displayed in spectral (FIG. 1, FIG. 3) or gel (FIG. 2, FIG. 4) views.

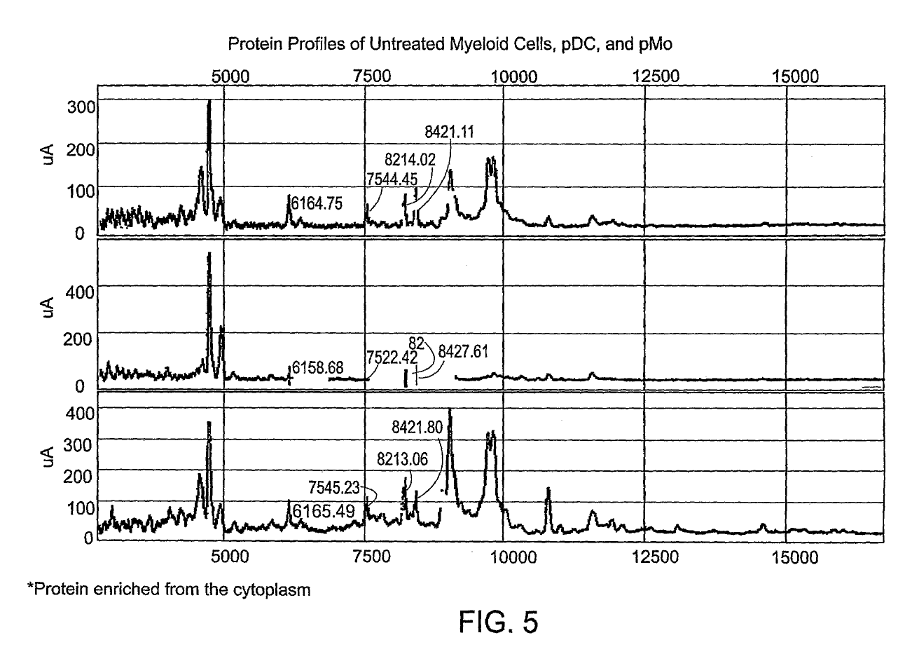

FIG. 5 illustrates the cytoplasmic protein profiles (signatures) from untreated/uninfected myeloid cells (Mx, top panel, (DC) mixture of dendritic cells, middle panel and (Mo) monocytes, bottom panel), DC, and Mo. Differences are observed in the individual profiles of DC and Mo.

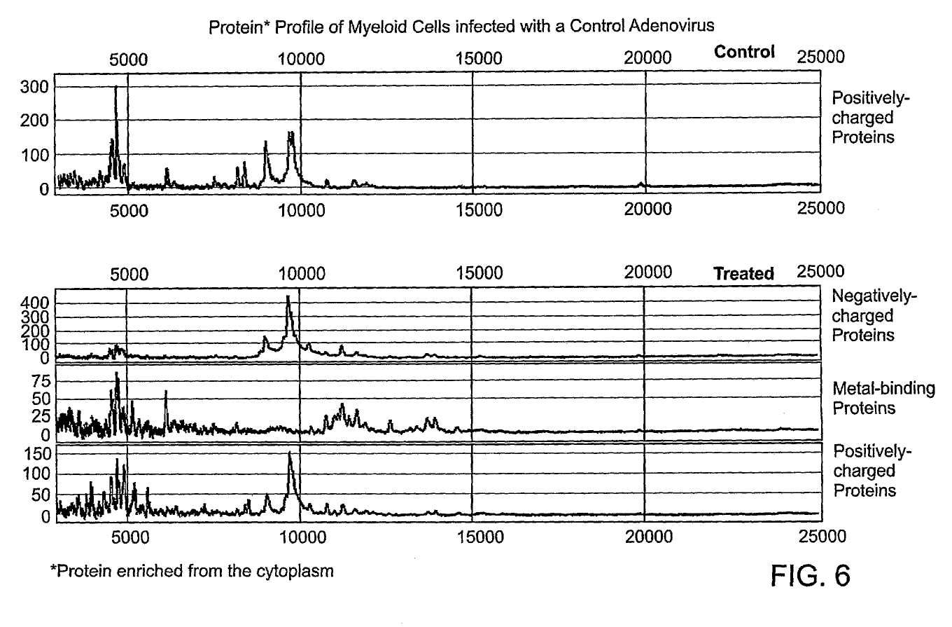

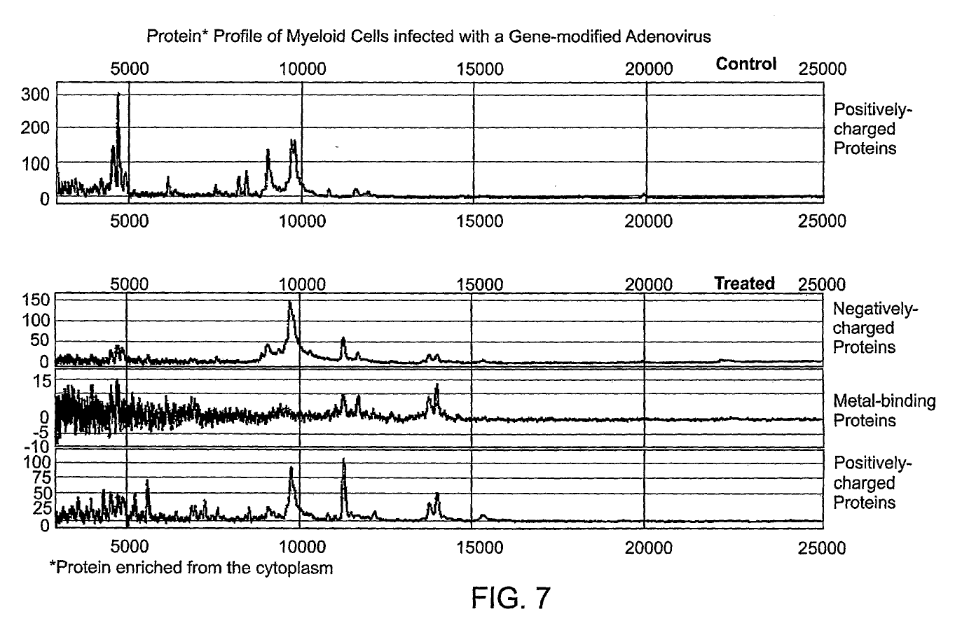

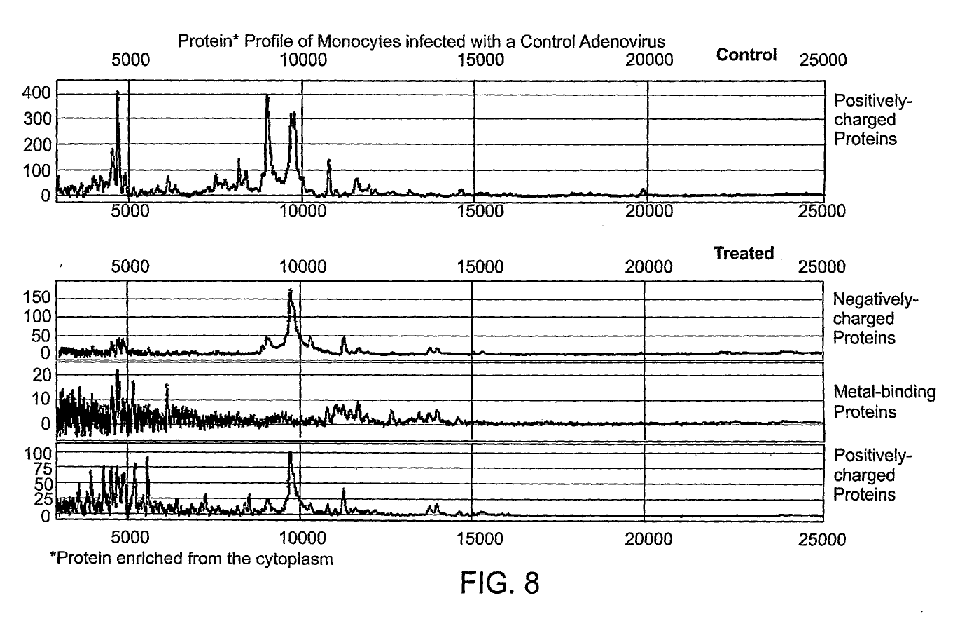

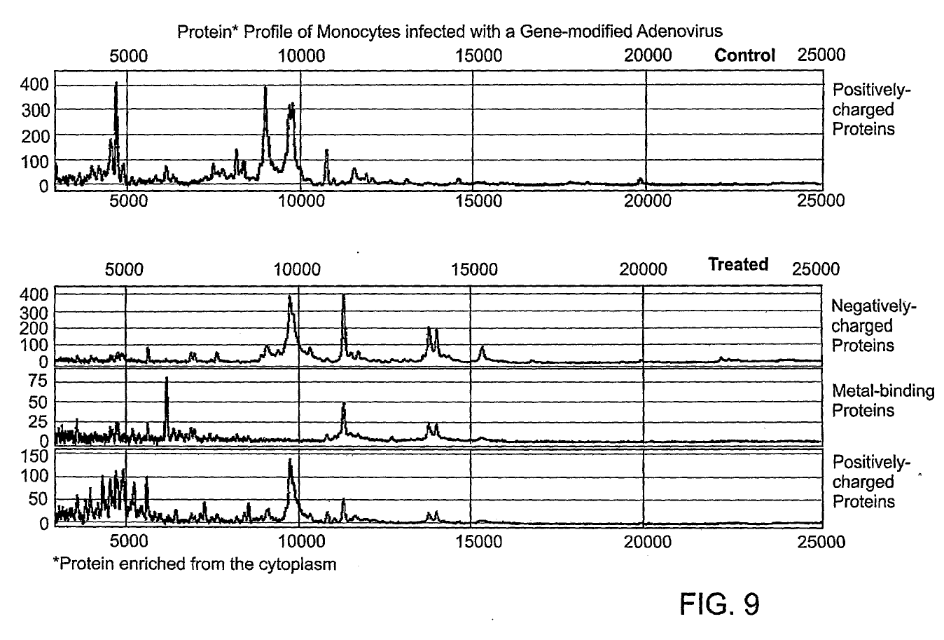

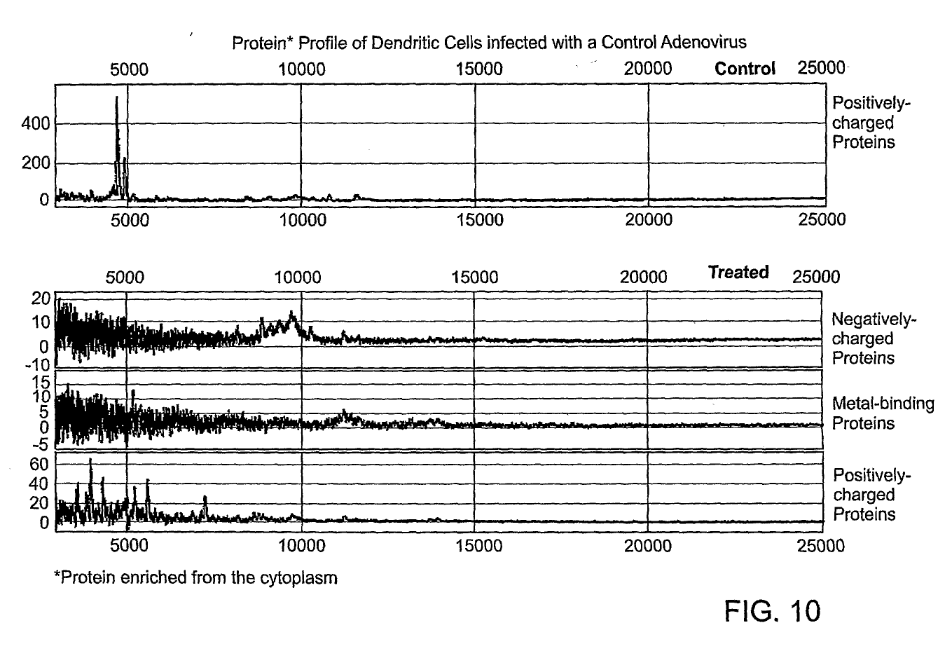

FIGS. 6-11 illustrate the cytoplasmic protein profiles (signatures) of Mx, Mo, and DC cultured in the presence of a control Adenovirus (FIGS. 6, 8, and 10) or in the presence of adenovirus with a single gene substitution (FIGS. 7, 9, and 11). The observed signatures show that APCs infected with wild-type and mutant adenovirus can be identified and distinguished using the present invention. Unique protein signatures can be obtained that can differentiate between viruses having one gene substitution/modification.

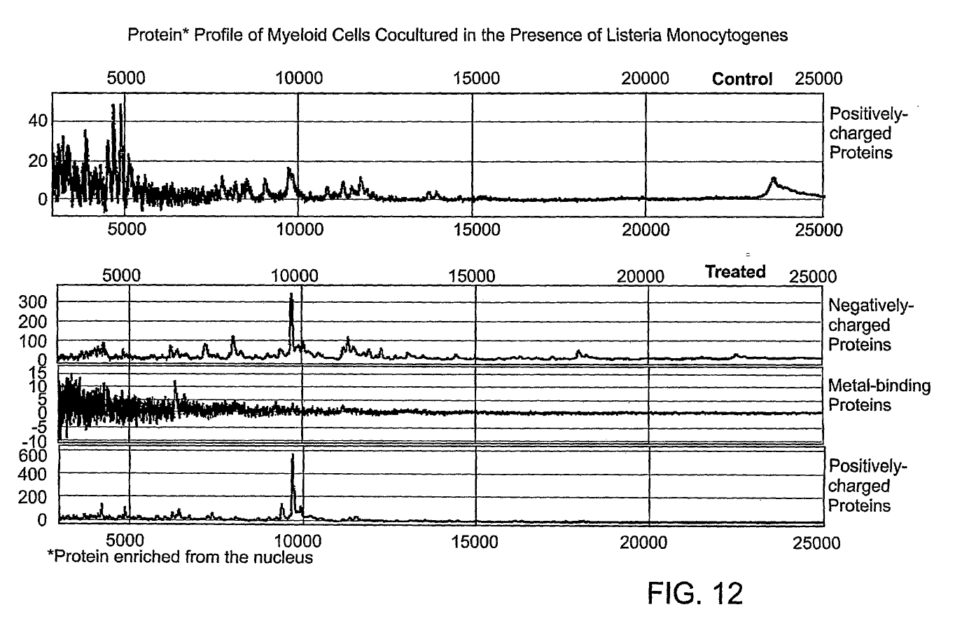

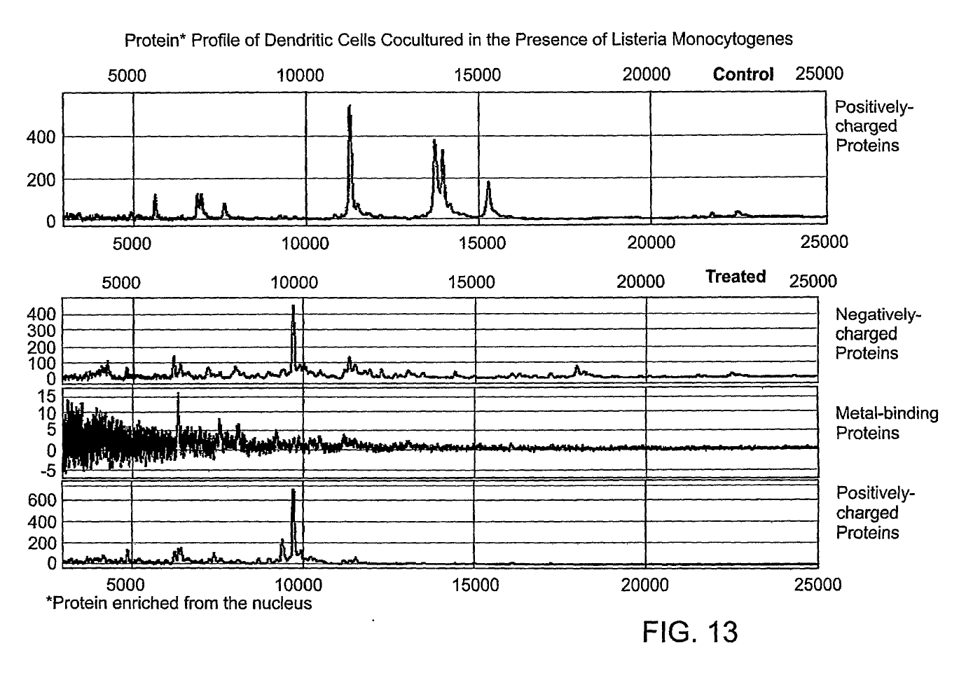

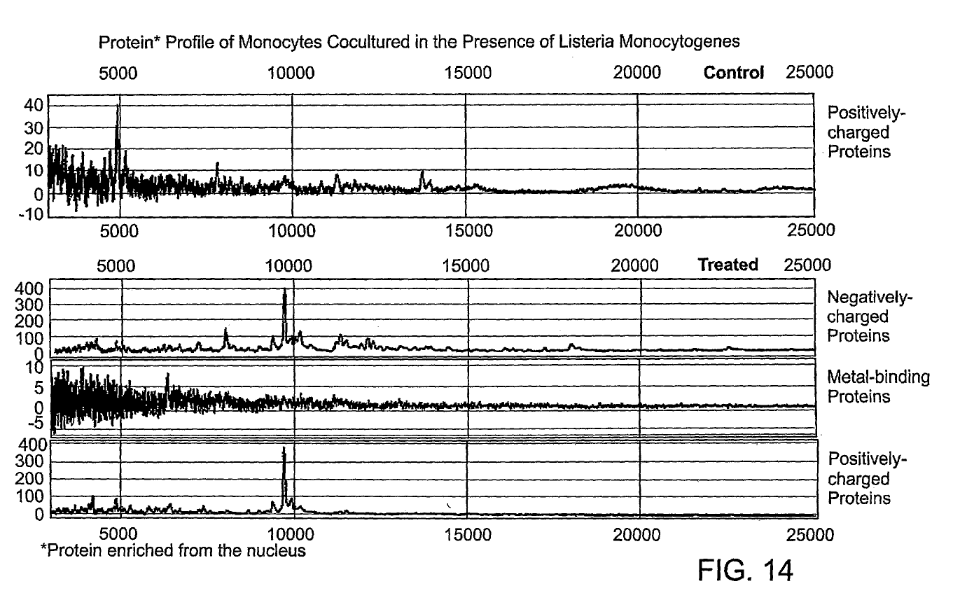

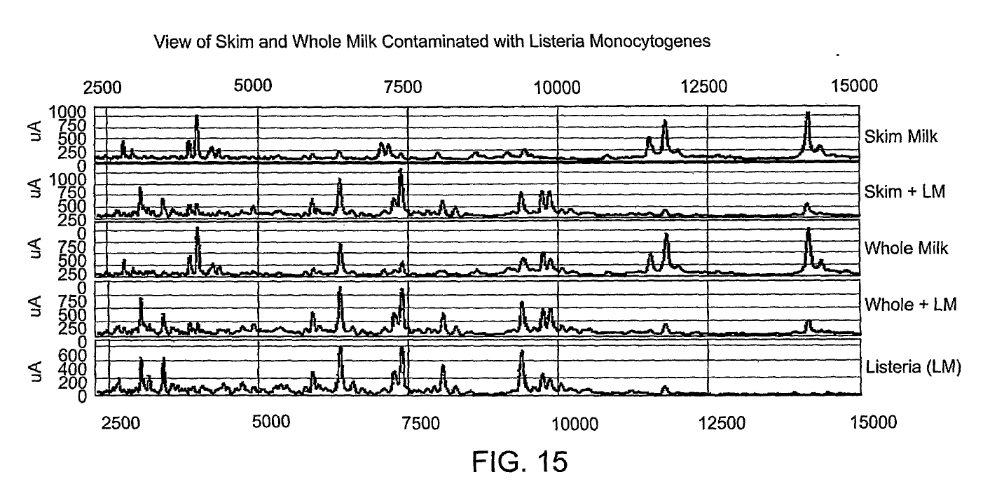

FIGS. 12-14 illustrate the protein profiles (signatures) obtained from nuclear protein extracts of Mx (FIG. 12), DC (FIG. 13) and Mo (FIG. 14) cocultured in the presence of Listeria monocytogenes. APCs cultured in the presence of other Gram positive and Gram negative bacteria also generate unique signatures that can be used to identify the microorganism cocultured with the APCs. The proteomic signature of Listeria alone, i.e., not cultured with APC (bottom panel of FIG. 15) is distinct from the three coculture signatures, suggesting that Listeria overgrowth did not occur thereby contaminating the APC cocultures.

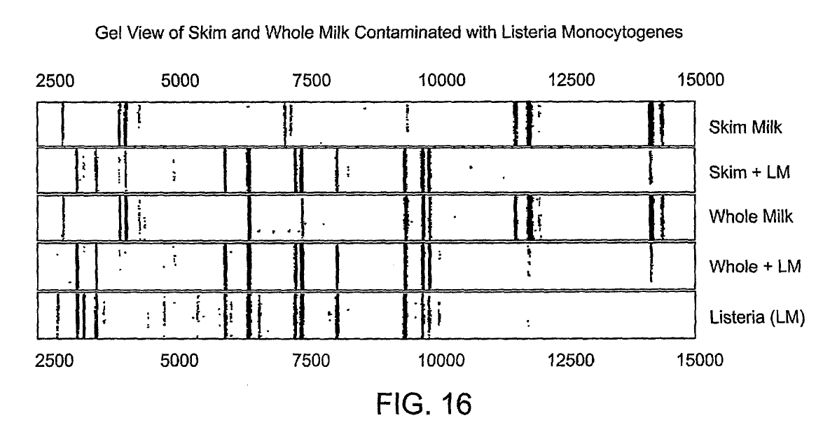

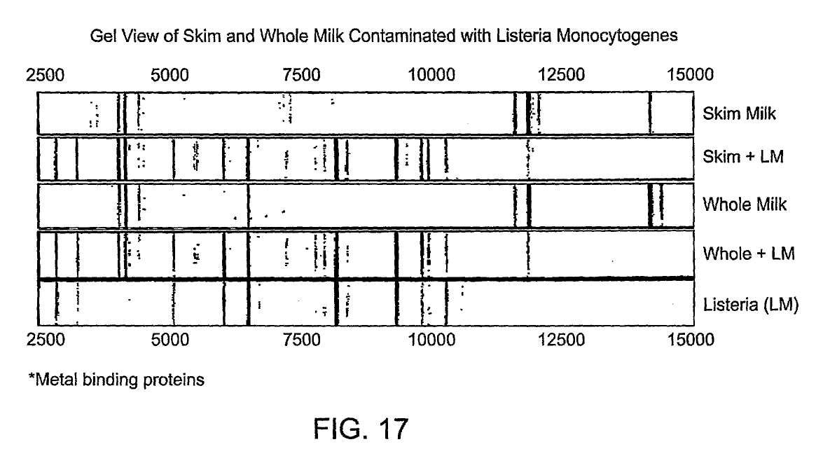

FIGS. 16-17 illustrate the spectroscopic profiles (signatures) of either skim or whole milk with Listeria contamination. FIG. 15 represents the spectral view of the results while FIGS. 16-17 represent the gel-views. The results demonstrate the ability of the present invention to detect the presence of unique pathogens in milk and other food stuffs, independent of APC detection methods.

### EXAMPLE FOUR

In addition to pathological agents, the diagnostic methods include the detection, diagnosis and staging of various cancers and genetic disorders. Cancers are detectable by numerous markers. Malignant cells often express antigens that are not found in normal cells; some of these antigens are found at the surface of the cell, for example CEA (chorioembryonic antigen) and differentially glycosylated (hypoglycosylated) MUC-1, are two well-known tumor associated antigens. MUC-1/DF-3 is overexpressed in the majority of human carcinomas, multiple myeloma, acute myelogenous leukemia, acute lymphoblastic leukemia, and follicular lymphoma among others. The antigen can initiate an HLA-restricted T cell response following presentation of the antigen by DC (see, Brossart et al., (2001) Cancer Res. September:61(18):6846-50. Other proteins upregulated in cancer cells include vascular endothelial growth factor (VEGF), Her-2/neu and hepsin. Intercellular adhesion molecule-1 (ICAM-1), vascular adhesion molecule-1 (VCAM-1) and E-Selectin (ELAM-1) play an important role in the complex series of events associated with inflammatory responses associated with cancer and tumor suppression.

In addition to pathological agents, the diagnostic methods include the detection, diagnosis and staging of various tumors and neoplasms. The invention can detect any of various malignant neoplasms characterized by the proliferation of anaplastic cells that tend to invade surrounding tissue and metastasize to new body sites. For example, APC's are cultured with the following cancers, to produce APC signatures of cancer-exposed cells: astrocytomas, gliomas, ependymomas, osteosarcoma, Ewing's sarcoma, retinoblastoma, bladder cancer, small and non-small cell lung cancer, oat cell lung cancer, pancreatic cancer, colorectal cancer, cervical cancer, endometrial cancer, vaginal cancer, ovarian cancer, cancers of the liver, acute lymphocytic leukemia, acute myelogenous leukemia, lymphoma, myeloma, basal cell carcinoma, melanoma, thyroid follicular cancer, bladder carcinoma, glioma, myelodysplastic syndrome, testicular cancer, stomach cancer, esophageal cancer, laryngeal cancer, squamous cell carcinoma, adenocarcinoma, leiomyosarcoma, urothelial carcinoma, breast cancer or prostate cancer.

APC, particularly CD2+ DC are cultured with primary or metastatic tumor cells obtained by biopsy, preferably cells taken from representative stages of tumor growth. Alternatively, APC are cultured with purified preparations of CEA (chorioembryonic antigen) or differentially glycosylated (hypoglycosylated) MUC-1, or other tumor associated antigens. The APC lysates are used to prepare cDNA which is then used to create arrays of APC reference standards for high throughput screens. Proteins upregulated in response to antigen contact are used to prepare antibodies, which are useful in immunological detection assays, e.g., ELISA, flow cytometry, FACS and multiplex assays. Arrays are prepared for each cancer type listed above, and preferably include each stage of the particular cancer type.

### EXAMPLE FIVE

Kits are developed for isolation of samples from subjects and from the environment.

For patient diagnostic uses, the kits include reagents and materials for obtaining and isolating blood samples from patients, as well as reagents and materials for enriching cells such as APC's, PBMC's and more preferably DC's, and for processing the cells into cytoplasmic, nuclear, or membrane fractions and optionally for processing larger proteins into smaller peptides.

Kits may further include chips or plates and reagents, appropriate for use with mass spectroscopy, such as those produced by Ciphergen for SELDI and Perkin Elmer for MALDI-O-TOF. The kits also include suitable instructions for use. In certain embodiments, the kits include one or more of the APC arrays mentioned above, e.g., for use in diagnosing and staging cancers, or for determining the agent of infection and progression of the infection, or for forensic analysis. Other kit components include controls such as reference proteins, used to calibrate the mass spectrometer. In still other embodiments, the kit includes albumin or high molecular weight proteins, and is used for enhancing the resolution of low molecular with proteins in the signatures (the albumin bump technique). Kits for use with patients are suitable for human and veterinary uses.

The following kits are provided herein: biodefense kits for identifying pathogens associated with bioweapons in the environment, and in exposed subjects; agricultural kits for sampling contamination of food dairy products and livestock; endocrine and metabolic kits for assessing endocrine and metabolic function in a subject; neurological kits for assessing degenerative changes; infectious disease kits for identifying pathogens in an exposed subject; prenatal kits for assessing fetal health; cancer kits for diagnosing and staging cancer progression in a subject and for monitoring chemotherapy regimens and disease progression; cardiovascular kits for detecting early signs of cardiac damage and ischemia and vessel occlusion; renal kits for detecting damage in the subject from i.e., contrast agents and chemotherapy drugs.

### EXAMPLE SIX

The potential threat to national security posed by terror attacks involving biological, chemical, nuclear, and radiological weapons is a serious international concern. One of the challenges facing public health officials responding to such an attack is a previously limited ability to diagnose individuals who have been exposed to these agents and do not show illness. Medical professionals and governmental officials all recognize that disease outbreaks—such as SARS in Asia and Canada, avian influenza in East Asia, and Ebola and Marburg virus in Africa—demonstrate that the speed of diagnosis and implementation of public health measures can mean the difference between an isolated outbreak and a global pandemic. The potential of terrorist attacks against agricultural targets (agro terrorism) is increasingly recognized as a national security threat. Agriculture has several characteristics that pose unique problems for managing the threat due to the fact that agricultural production is geographically disbursed and in unsecured environments. Livestock are frequently concentrated in confined locations, and then transported and comingled with other herds. Foot and mouth disease (FMD) outbreaks in Europe, and the recent detection of a second case of BSE in the United States underscore the importance of detecting disease in livestock in a rapid and accurate manner. Foods, such as milk are stored in accessible areas that can be purposefully contaminated. Pest and disease outbreaks can quickly halt economically important exports. Effective detection depends on a heightened sense of awareness, and on the ability to rapidly determine the level of threat by the ability to screen livestock, as well as foods rapidly. Lessons from past disease outbreaks, show that the speed of detection and diagnosis can determine the difference between an isolated incident and wide spread disease.

The present invention provides comprehensive genomic, bioinformatics, functional genomics, and immune cell (APC-based) proteomic approaches for the detection and monitoring of bioterrorism agents, and the qualitative and quantitative assessment of infectious agents and their effects on the immune system of a subject. These approaches also provide a critical resource for the scientific community that could lead to the discovery and identification of novel targets for the next generation of drugs, vaccines, diagnostics and immunotherapeutics. Most importantly, these approaches are rapid and accurate.

In one aspect, a Proteomic Pathogen Reference Library (PPRL), that is electronically searchable, is constructed. The PPRL includes records of immune surveillance cells (APC) that have been contacted with the individual and combinations of the toxins and pathogens on the CDC Bioterrorism Agents and Diseases List. The records provide reference signatures for positive exposure of APCs to these agents. The PPRL is useful in detecting exposure of biological/chemical pathogens in human subjects, and can also be used to detect exposure of other mammals such as livestock. The PPRL also provides a reference for screening food(s) for bacterial and chemical pathogens that could be potentially introduced accidentally or deliberately into the food supply.

Signatures of APC are obtained, using cells that have been contacted with the toxins and pathogens on the CDC Bioterrorism Agents and Diseases List. These include: Anthrax toxins (Bacillus anthracis); Arenaviruses; Bacillus anthracis (anthrax); Clostridium botulinum toxin); Brucella species; Burkholderia mallei; Burkholderia pseudomallei (melioidosis); Chlamydia psittaci; Cholera toxin; Clostridium botulinum toxin (botulism); Clostridium perfringens; Ebola virus hemorrhagic fever; Emerging infectious diseases such as Nipah virus and hantavirus; Epsilon toxin of Clostridium perfringens; Escherichia coli O157:H7 (E. coli); Food safety threats (e.g., Salmonella species, Escherichia coli O157:H7, Shigella); Francisella tularensis (tularemia); Glanders (Burkholderia mallei); Lassa fever; Marburg virus hemorrhagic fever; Melioidosis (Burkholderia pseudomallei); Psittacosis (Chlamydia psittaci); Q fever (Coxiella burnetii); Ricin toxin from Ricinus communis (castor beans); Rickettsia prowazekii (typhus fever); Salmonella species (salmonellosis); Salmonella Typhi (typhoid fever); Salmonellosis (Salmonella species); Shigella (shigellosis); Shigellosis (Shigella); Smallpox (variola major); Staphylococcal enterotoxin B; Typhoid fever (Salmonella Typhi); Typhus fever (Rickettsia prowazekii); Variola major (smallpox); Vibrio cholerae (cholera); Viral encephalitis (alphaviruses [e.g., Venezuelan equine encephalitis, eastern equine encephalitis, western equine encephalitis]); Viral hemorrhagic fevers (filoviruses [e.g., Ebola, Marburg] and arenaviruses [e.g., Lassa, Machupo]); Water safety threats (e.g., Vibrio cholerae, Cryptosporidium parvum); and Yersinia pestis (plague).

Signatures of the exposed APC are digitized and recorded on computer readable media as an individual data record. In one aspect, proteomic signatures of antigen exposed intact APC (each cell subtype) are obtained using, for example SELDI, MALDI-O-TOF, and MALDI-TOF. Metadata is provided with the data record, including APC cell type, toxin or pathogen, buffer composition and materials/methods for obtaining the particular APC signature, donor information, and other pertinent medical and etiological information. This is a reference data set. The reference data set is obtained for each APC cell type and each toxin or pathogen, and collectively comprise the reference data records in a Proteomic Pathogen Reference Library. The PPRL includes an expert system for parsing the reference data records, excluding and including individual data sets based on user defined criteria, and a pattern recognition routine to match data sets, and particularly the signatures of the exposed APC, to the signature of the unknown data set having an input subject derived sample, based on similarity of proteomic signatures.

To use the PPRL, a medical professional obtains a sample of blood from a subject suspected of exposure to a CDC Bioterrorism Agent. The APC are isolated from the blood sample and are separated by APC cell subtype, e.g., by FACS for antigenic markers such as CD2+ DC, or CD4+ T-cells, etc. Each APC subtype is used to obtain whole cell mass spec proteomic signatures. The signatures are digitized and uploaded to a computer program having algorithms that compile the proteomic signature and allow the medical professional to add metadata relevant to the sample, thereby producing an unknown data set. The computer program allows the medical professional to further compile the data set with user defined information into a search query, and communicates the search query to the PPRL, preferably over a network. The expert system parses the reference data records with the unknown data set as described. Potential matches, defined as reference data sets having agreement with the unknown data set to a particular defined confidence interval, are returned to the medical professional over the network. The medical professional is thus able to confirm or exclude exposure of the subject to the various CDC Bioterrorism Agents.

In one aspect, the PPRL is networked with one or more of the following CDC programs: the Active Bacterial Core Surveillance (ABCs); the Gonococcal Isolate Surveillance Project (GISP); the National Antimicrobial Resistance Monitoring System: Enteric Bacteria (NARMS:EB); the National Electronic Disease Surveillance System (NEDSS); the Health Information and Surveillance Systems Board (HISSB); the National Nosocomial Infections Surveillance System (NNIS); the Intensive Care Antimicrobial Resistance Epidemiology (ICARE); and the Surveillance of Emerging Antimicrobial Resistance Connected to Healthcare (SEARCH) program. In one embodiment, a query resulting in a positive identification of a CDC Bioterrorism Agent in a subject sample, alerts one or more of these program groups to the positive identification. In another embodiment, an alert is sent to the Department of Homeland Security. In yet another embodiment, an alert is sent to hospitals within a specified geographic area, e.g., proximal to the site of detection.

### EXAMPLE SEVEN

The PPRL includes records of immune surveillance cells (APC) that have been contacted with bacterial pathogens, including the common disease producing pathogens, nosocomial pathogens and drug resistant pathogens. The records provide reference signatures for positive exposure of APCs to these bacterial pathogens. The PPRL is useful in detecting exposure of bacterial pathogens in human subjects, and can also be used to detect exposure of other mammals such as livestock.

Signatures of APC are obtained, using cells that have been contacted with the following pathogenic bacteria. These include: Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria resistant to penicillins, tetracyclines, spectinomycin, and fluoroquinolones; Methicillin-resistant Staphylococcus Aureus (MRSA); drug-resistant Streptococcus pneumoniae; fluoroquinolone and other drug resistant Salmonella serogroup Typhi; Vancomycin-Intermediate/Resistant Staphylococcus aureus; and Vancomycin-resistant Enterococci, as well as other multi-drug resistant strains of bacteria.

Signatures of the pathogen exposed APC are digitized and recorded on computer readable media as an individual data record. In one aspect, proteomic signatures of antigen exposed intact APC (each cell subtype) are obtained using, for example SELDI, MALDI-O-TOF, and MALDI-TOF. Metadata is provided with the data record, including APC cell type, toxin or pathogen, buffer composition and materials/methods for obtaining the particular APC signature, donor information, and other pertinent medical and etiological information. In another aspect, APC series are obtained from reference subjects having a bacterial infection, that have been in various stages of infection, i.e., initial localized infection, disseminated infection, moderate sepsis, severe sepsis, SIRS, septic shock, and multiple organ dysfunction syndrome (MODS). This is a reference data set. The reference data set is obtained for each APC cell type and each bacterial pathogen and infection stage, and collectively comprise the reference data records in a Proteomic Pathogen Reference Library. The PPRL includes an expert system for parsing the reference data records, excluding and including individual data sets based on user defined criteria, and a pattern recognition routine to match data sets, and particularly the signatures of the exposed APC, to the signature of the unknown data set having an input subject derived sample, based on similarity of proteomic signatures.

To use the PPRL, a medical professional obtains a sample of blood from a subject suspected of exposure to a bacterial pathogen. The APC are isolated from the blood sample and are separated by APC cell subtype, e.g., by FACS for antigenic markers such as CD2+ DC, or CD4+ T-cells, etc. Each APC subtype is used to obtain whole cell mass spec proteomic signatures. The signatures are digitized and uploaded to a computer program having algorithms that compile the proteomic signature and allow the medical professional to add metadata relevant to the sample, thereby producing an unknown data set. The computer program allows the medical professional to further compile the data set with user defined information into a search query, and communicates the search query to the PPRL, preferably over a network. The expert system parses the reference data records with the unknown data set as described. Potential matches, defined as reference data sets having agreement with the unknown data set to a particular defined confidence interval, are returned to the medical professional over the network. The medical professional is thus able to confirm or exclude exposure of the subject to the various bacterial pathogens, and discern the stage of infection. Further assays such as amplification of antibiotic resistance genes by PCR can confirm or exclude bacteria that may be resistant to specific drugs, thus aiding the course of therapy.

### EXAMPLE EIGHT

A cancer proteomic reference library is created that includes records of immune surveillance cells (APC) that have been obtained from patients having different types of cancer, and at various disease stages of these cancers. The records provide reference signatures for positive exposure of APCs to various cancers at different stages of disease progression. The library is useful in diagnosing the presence of cancer in a test subject, as well as identifying the cancer type and stage.

The following cancer types each result in specific APC responses, and are amenable to detection using the techniques described: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; AIDS-Related Cancers; AIDS-Related Lymphoma; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Non-Hodgkin's During Pregnancy; Lymphoma, Primary Central Nervous System; Macroglobulinemia, Waldenström's; Male Breast Cancer; Malignant Mesothelioma, Adult; Malignant Mesothelioma, Childhood; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma and Thymic Carcinoma Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma, Childhood; Vulvar Cancer; Waldenström's Macroglobulinemia; and Wilms' Tumor.

Signatures of the exposed APC's are digitized and recorded on computer readable media as an individual data record. In one aspect, proteomic signatures of cancer exposed intact APC (each cell subtype) are obtained using, for example SELDI, MALDI-O-TOF, and MALDI-TOF. Metadata is provided with the data record, including APC cell type, histological information about the cancer, buffer composition and materials/methods for obtaining the particular APC signature, donor information, and other pertinent medical and etiological information. In another aspect, APC series are obtained from reference subjects having a cellular proliferative disease, that have been in various stages of the disease, i.e., stage 1, stage 2, stage 3 or stage 4, etc. This is a reference data set. The reference data set is obtained for each APC cell type and each cancer type and disease stage, and collectively comprise the reference data records in a Proteomic Cancer Reference Library. The PCRL includes an expert system for parsing the reference data records, excluding and including individual data sets based on user defined criteria, and a pattern recognition routine to match data sets, and particularly the signatures of the exposed APC, to the signature of the unknown data set having an input subject derived sample, based on similarity of proteomic signatures.

To use the PCRL, a medical professional obtains a sample of blood from a subject under study. The subject may not have cancer and the procedure is simply a screen for disease, or the subject may be suspected of having a cancer due to genetic predisposition or preliminary medical examination, or the subject may have a confirmed cancer and the procedure is designed to monitor changes in the disease. The APC are isolated from the blood sample and are separated by APC cell subtype, e.g., by FACS for antigenic markers such as CD2+ DC, or CD4+ T-cells, etc. Each APC subtype is used to obtain whole cell mass spec proteomic signatures. The signatures are digitized and uploaded to a computer program having algorithms that compile the proteomic signature and allow the medical professional to add metadata relevant to the sample, thereby producing an unknown data set. The computer program allows the medical professional to further compile the data set with user defined information into a search query, and communicates the search query to the PCRL, preferably over a network. The expert system parses the reference data records with the unknown data set as described. Potential matches, defined as reference data sets having agreement with the unknown data set to a particular defined confidence interval, are returned to the medical professional over the network. The medical professional is thus able to ascertain if the subject has an APC signature that indicates the presence of various cancers, and discern the stage of disease.

### EQUIVALENTS

From the foregoing detailed description of the specific embodiments of the invention, it should be apparent that unique detection methodologies have been described. Although particular embodiments have been disclosed herein in detail, this has been done by way of example for purposes of illustration only, and is not intended to be limiting with respect to the scope of the appended claims which follow. In particular, it is contemplated by the inventors that various substitutions, alterations, and modifications may be made to the invention without departing from the spirit and scope of the invention as defined by the claims. For instance, the choice of spectrum, or the APC used in the detection process is believed to be matter of routine for a person of ordinary skill in the art with knowledge of the embodiments described herein.

### REFERENCES

All U.S. patents and other references cited herein are hereby incorporated herein by reference in their entirety.
Arad, G., Levy, R., & Kaempfer, R. 2002. Superantigen concomitantly induces Th1 cytokine genes and the ability to shut off their expression on re-exposure to superantigen. Immunol. Lett, 82(1-2): 75-78.
Chapes, S. K., Beharka, A. A., Hart, M. E., Smeltzer, M. S., & Iandolo, J. J. 1994. Differential RNA regulation by staphylococcal enterotoxins A and B in murine macrophages. J Leukoc. Biol, 55(4): 523-529.
Chaussabel D, Semnani R T, McDowell M A, Sacks D, Sher A, Nutman T B (2003): Unique gene expression profiles of human macrophages and dendritic cells to phylogenetically distinct parasites. Blood 102:672-81.
Clemens, D. L., Lee, B. Y., & Horwitz, M. A. 2004. Virulent and avirulent strains of Francisella tularensis prevent acidification and maturation of their phagosomes and escape into the cytoplasm in human macrophages. Infect. Immun., 72(6): 3204-3217.
Corner, J. E., Galindo, C. L., Chopra, A. K., & Peterson, J. W. 2005. GeneChip analyses of global transcriptional responses of murine macrophages to the lethal toxin of Bacillus anthracis. Infect. Immun., 73(3): 1879-1885.
Crawford K, Stark A, Kitchens B, Sternheim K, Pantazopoulos V, Triantafellow E, Wang Z, Vasir B, Larsen C E, Gabuzda D, Reinherz E, Alper C A (2003): CD2 engagement induces dendritic cell activation: implications for immune surveillance and T-cell activation. Blood 102:1745-52. Jantsch J, Cheminay C, Chakravortty D, Lindig T, Hem J, Hensel M (2003): Intracellular activities of Salmonella enterica in murine dendritic cells. 5:933-45.
Deringer, J. R., Ely, R. J., Stauffacher, C. V., & Bohach, G. A. 1996. Subtype-specific interactions of type C staphylococcal enterotoxins with the T-cell receptor. Mol Microbiol, 22(3): 523-534.
Frandji, P., Tkaczyk, C., Oskeritzian, C., David, B., Desaymard, C., & Mecheri, S. 1996. Exogenous and endogenous antigens are differentially presented by mast cells to CD4+ T lymphocytes. Eur. J. Immunol., 26(10): 2517-2528.
Hernychova, L., Kovarova, H., Macela, A., Kroca, M., Krocova, Z., & Stulik, J. 1997. Early consequences of macrophage-Francisella tularensis interaction under the influence of different genetic background in mice. Immunol. Lett, 57(1-3): 75-81.
Hofer 5, Rescigno M, Granucci F, Citterio 5, Francolini M, Ricciardi-Castagnoli P (2001): Differential activation of NF-kappa B subunits in dendritic cells in response to Gram-negative bacteria and to lipopolysaccharide. Microbes and Infection/Institut Pasteur 3:259-65.
LeClaire, R. D., Kell, W. M., Sadik, R. A., Downs, M. B., & Parker, G. W. 1995. Regulation of staphylococcal enterotoxin B-elicited nitric oxide production by endothelial cells. Infect. Immun., 63(2): 539-546.
Lemay, S., Mao, C., & Singh, A. K. 1996. Cytokine gene expression in the MRL/lpr model of lupus nephritis. Kidney Int, 50(1): 85-93.
Mascarell, L. & Truffa-Bachi, P. 2004. T lymphocyte activation initiates the degradation of the CD62L encoding mRNA and increases the transcription of the corresponding gene. Immunol. Lett, 94(1-2): 115-122.
Moon, Y. & Pestka, J. J. 2003. Cyclooxygenase-2 mediates interleukin-6 upregulation by vomitoxin (deoxynivalenol) in vitro and in vivo. Toxicol. Appl Pharmacol, 187(2): 80-88.
Novellino, P. S., Trejo, Y. G., Beviacqua, M., Bordenave, R. H., & Rumi, L. S. 2000. Regulation of HLA-DR antigen in monocytes from colorectal cancer patients by in vitro treatment with human recombinant interferon-gamma. J Investig. Allergol. Clin Immunol., 10(2): 90-93.
Pulendran B, Kumar P, Cutler C W, Mohamadzadeh M, Van_Dyke T, Banchereau J (2001): Lipopolysaccharides from distinct pathogens induce different classes of immune responses in vivo. Journal of Immunology (Baltimore, Md.: 1950) 167:5067-76.
Rachlis, A., Watson, J. L., Lu, J., & McKay, D. M. 2002. Nitric oxide reduces bacterial superantigen-immune cell activation and consequent epithelial abnormalities. J Leukoc. Biol, 72(2): 339-346.
Schlueter, A. J., Krieg, A. M., de Vries, P., & Li, X. 2001. Type I interferon is the primary regulator of inducible Ly-6C expression on T cells. J Interferon Cytokine Res, 21(8): 621-629.
Schmitz, J. & Radbruch, A. 1992. Distinct antigen presenting cell-derived signals induce TH cell proliferation and expression of effector cytokines. Int Immunol., 4(1): 43-51.
Sundquist M, Rydstrom A, Wick M J (2004): Immunity to Salmonella from a dendritic point of view. Cell Microbiol 6:1-li. Appelmelk B J, van_Die I, van_Vliet S J, Vandenbroucke_Grauls C M, Geijtenbeek T B, van_Kooyk Y (2003): Cutting edge: carbohydrate profiling identifies new pathogens that interact with dendritic cell-specific ICAM-3-grabbing nonintegrin on dendritic cells. Journal of Immunology (Baltimore, Md.: 1950) 170:1635-9.
Szabo, G., Mandrekar, P., & Catalano, D. 1995. Inhibition of superantigen-induced T cell proliferation and monocyte IL-1 beta, TNF-alpha, and IL-6 production by acute ethanol treatment. J Leukoc. Biol, 58(3): 342-350.
Szebeni, J. & Sykes, M. 1996. CAG repeat mutation in the mouse IL-2 gene enables concurrent assessment of T- and B-cell-specific gene expression by northern analysis. Exp. Clin Immunogenet., 13(2): 117-119.
Takahashi, M., Takahashi, M., Shinohara, F., Takada, H., & Rikiishi, H. 2001. Effects of superantigen and lipopolysaccharide on induction of CD80 through apoptosis of human monocytes. Infect. Immun., 69(6): 3652-3657.
Thomassen, M. J., Ahmad, M., Barna, B. P., Antal, J., Wiedemann, H. P., Meeker, D. P., Klein, J., Bauer, L., Gibson, V., Andresen, S., &. 1991. Induction of cytokine messenger RNA and secretion in alveolar macrophages and blood monocytes from patients with lung cancer receiving granulocyte-macrophage colony-stimulating factor therapy. Cancer Res, 51(3): 857-862.
Tucker, A. E., Salles, I. I., Voth, D. E., Ortiz-Leduc, W., Wang, H., Dozmorov, I., Centola, M., & Ballard, J. D. 2003. Decreased glycogen synthase kinase 3-beta levels and related physiological changes in Bacillus anthracis lethal toxin-treated macrophages. Cell Microbiol, 5(8): 523-532.
Wakita, H., Tokura, Y., Furukawa, F., & Takigawa, M. 1995. Staphylococcal enterotoxin B upregulates expression of ICAM-1 molecules on IFN-gamma-treated keratinocytes and keratinocyte cell lines. J Invest Dermatol., 105(4): 536-542.
Wang, Y., Wu, T. R., Cai, S., Welte, T., & Chin, Y. E. 2000. Stat1 as a component of tumor necrosis factor alpha receptor 1-TRADD signaling complex to inhibit NF-kappaB activation. Mol Cell Biol, 20(13): 4505-4512.
Wrighton, C. J., Hofer-Warbinek, R., Moll, T., Eytner, R., Bach, F. H., & de Martin, R. 1996. Inhibition of endothelial cell activation by adenovirus-mediated expression of I kappa B alpha, an inhibitor of the transcription factor NF-kappa B. J. Exp. Med, 183(3): 1013-1022.
Yang, Y., Kim, D., & Fathman, C. G. 1998. Regulation of programmed cell death following T cell activation in vivo. Int Immunol., 10(2): 175-183.
Zembala, M., Czupryna, A., Wieckiewicz, J., Jasinski, M., Pryjma, J., Ruggiero, I., Siedlar, M., & Popiela, T. 1993. Tumour-cell-induced production of tumour necrosis factor by monocytes of gastric cancer patients receiving BCG immunotherapy. Cancer Immunol. Immunother., 36(2): 127-132.
Zoja, C., Corna, D., Camozzi, D., Cattaneo, D., Rottoli, D., Batani, C., Zanchi, C., Abbate, M., & Remuzzi, G. 2002. How to fully protect the kidney in a severe model of progressive nephropathy: a multidrug approach. J Am Soc Nephrol, 13(12): 2898-2908.

We claim:

1. A method for identifying exposure of a mammalian subject to a pathogen or toxin comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to the pathogen or toxin; wherein congruency between the subject signature and the reference signature indicates exposure of the subject to the pathogen or toxin.

2. The method of claim 1, wherein the pathogen or toxin is bacterial.

3. The method of claim 2, wherein the bacterial pathogen is selected from the group consisting of: Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria resistant to penicillins, tetracyclines, spectinomycin, and fluoroquinolones; Methicillin-resistant Staphylococcus Aureus (MRSA); drug-resistant Streptococcus pneumoniae; fluoroquinolone and other drug resistant Salmonella serogroup Typhi; Vancomycin-Intermediate/Resistant Staphylococcus aureus; and Vancomycin-resistant Enterococci.

4. The method of claim 1, wherein the pathogen or toxin is selected from the group consisting of: Anthrax toxin; Arenavirus; Bacillus anthracis (anthrax); Clostridium botulinum toxin); Brucella species; Burkholderia mallei; Burkholderia pseudomallei (melioidosis); Chlamydia psittaci; Cholera toxin; Clostridium botulinum toxin (botulism); Clostridium perfringens; Ebola virus hemorrhagic fever; Nipah virus; hantavirus; Epsilon toxin of Clostridium perfringens; Escherichia coli including strain O157:H7; Shigella; Francisella tularensis; Glanders (Burkholderia mallei); Lassa fever; Marburg virus hemorrhagic fever; Melioidosis (Burkholderia pseudomallei); Psittacosis (Chlamydia psittaci); Q fever (Coxiella burnetii); Ricin toxin from Ricinus communis (castor beans); Rickettsia prowazekii; Salmonella Typhi and other Salmonella species; Shigella; Smallpox; Staphylococcal enterotoxin B; Typhus fever; Variola major; Vibrio cholerae (cholera); Viral encephalitis; alphaviruses such as Venezuelan equine encephalitis, eastern equine encephalitis, and western equine encephalitis; Filoviruses; Arenaviruses such as Lassa, and Machupo; Vibrio cholerae; Cryptosporidium parvum; and Yersinia pestis.

5. The method of claim 1, wherein the pathogen or toxin is prion.

6. A method of detecting pathogen or toxin contamination in a sample comprising: obtaining a sample; incubating the sample for a period of time with a population of naïve antigen presenting cells thereby contacting the antigen presenting cells with the sample; isolating and purifying the sample contacted antigen presenting cells; obtaining a proteomic signature for the sample contacted antigen presenting cells; and comparing the proteomic signature from the sample contacted antigen presenting cells to at least one reference signature, the reference signature comprising a proteomic signature for reference antigen presenting cells that have been exposed to the pathogen or toxin; wherein congruency between the sample contacted signature and the reference signature indicates exposure of the subject to the pathogen or toxin.

7. The method of claim 6, wherein the pathogen or toxin is selected from the group consisting of: Anthrax toxin; Arenavirus; Bacillus anthracis (anthrax); Clostridium botulinum toxin); Brucella species; Burkholderia mallei; Burkholderia pseudomallei (melioidosis); Chlamydia psittaci; Cholera toxin; Clostridium botulinum toxin (botulism); Clostridium perfringens; Ebola virus hemorrhagic fever; Nipah virus; hantavirus; Epsilon toxin of Clostridium perfringens; Escherichia coli including strain O157:H7; Shigella; Francisella tularensis; Glanders (Burkholderia mallei); Lassa fever; Marburg virus hemorrhagic fever; Melioidosis (Burkholderia pseudomallei); Psittacosis (Chlamydia psittaci); Q fever (Coxiella burnetii); Ricin toxin from Ricinus communis (castor beans); Rickettsia prowazekii; Salmonella Typhi and other Salmonella species; Shigella; Smallpox; Staphylococcal enterotoxin B; Typhus fever; Variola major; Vibrio cholerae (cholera); Viral encephalitis; alphaviruses such as Venezuelan equine encephalitis, eastern equine encephalitis, and western equine encephalitis; Filoviruses; Arenaviruses such as Lassa, and Machupo; Vibrio cholerae; Cryptosporidium parvum; and Yersinia pestis.

8. The method of claim 6, wherein the sample is a food product.

9. A method of diagnosing infection in a mammalian subject from a bacterial pathogen comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to a bacterial pathogen; wherein congruency between the subject signature and the reference signature indicates an active bacterial infection of the subject by the pathogen.

10. The method of claim 9, wherein the bacterial pathogen is selected from the group consisting of: Bacillus; Bordetella; Borrelia; Campylobacter; Clostridium; Corynebacterium; Enterococcus; Escherichia; Francisella; Haemophilus; Helicobacter; Legionella; Listeria; Mycobacterium; Neisseria; Pseudomonas; Salmonella; Shigella; Staphylococcus; Streptococcus; Treponema; Vibrio; Yersinia; Neisseria resistant to penicillins, tetracyclines, spectinomycin, and fluoroquinolones; Methicillin-resistant Staphylococcus Aureus (MRSA); drug-resistant Streptococcus pneumoniae; fluoroquinolone and other drug resistant Salmonella serogroup Typhi; Vancomycin-Intermediate/Resistant Staphylococcus aureus; and Vancomycin-resistant Enterococci.

11. The method of claim 9, further comprising assaying the blood of the mammalian subject for the presence of biomarkers for sepsis.

12. The method of claim 11, wherein the biomarkers for sepsis are selected from the group consisting of: D-dimer; apolipoprotein A1; beta-2 microglobulin; C-reactive protein; epidermal growth factor; endothelin-1; eotaxin; Factor VII; fibroblast growth factor-9; basic fibroblast growth factor; fibrinogen; granulocyte chemotactic protein-2; granulocyte-macrophage colony stimulating factor; growth hormone; glutathione S-transferase; gamma interferon; IgA; IL-10; IL-11; IL-12p70; IL-17; IL-18; IL-1beta; IL-2; IL-3; IL-4; IL-5; IL-6; IL-7; insulin; gamma interferon inducible protein 10; KC; leptin; leukemia inhibitory factor; lymphotactin; monocyte chemoattractant protein-1/JE; monocyte chemoattractant protein-3; monocyte chemoattractant protein-5; macrophage colony stimulating factor; macrophage derived chemokine; macrophage inflammatory protein-1 alpha; macrophage inflammatory protein-1 beta; macrophage inflammatory protein-1 gamma; macrophage inflammatory protein-2; macrophage inflammatory protein-3 beta; myoglobin; oncostatin M; RANTES; stem cell factor; aspartate amino transferase; tissue inhibitor metalloproteinase-1; tumor necrosis factor-alpha; tissue factor; thrombopoietin; vascular cell adhesion molecule-1; vascular endothelial growth factor; and von Willebrand factor.

13. The method of claim 11, wherein the biomarkers for sepsis are acute phase proteins.

14. A method of diagnosing cancer in a mammalian subject comprising: obtaining from the mammal a sample of fluid, the sample having antigen presenting cells; purifying the subject antigen presenting cells from the fluid; obtaining a subject proteomic signature for the subject antigen presenting cells; and comparing the proteomic signature from the subject antigen presenting cells to at least one reference signature, the reference signature comprising a proteomic signature for reference antigen presenting cells that have been exposed to a cancer; wherein congruency between the subject signature and the reference signature indicates the subject has the cancer.

15. The method of claim 14, wherein the fluid is selected from the group consisting of: blood, plasma, bone marrow, pericardial, pleural, ascitic, and synovial fluids, cerebrospinal fluids, sputum, urine, and lymphatic fluids.

16. The method of claim 14, wherein the cancer is selected from the group consisting of: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; AIDS-Related Cancers; AIDS-Related Lymphoma; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Non-Hodgkin's During Pregnancy; Lymphoma, Primary Central Nervous System; Macroglobulinemia, Waldenström's; Male Breast Cancer; Malignant Mesothelioma, Adult; Malignant Mesothelioma, Childhood; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma and Thymic Carcinoma Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma, Childhood; Vulvar Cancer; Waldenström's Macroglobulinemia; and Wilms' Tumor.

17. A system comprising: a processor, memory, user input device, output device and computer readable media having, a reference data set comprising a plurality of reference data records, each reference data record further comprising the proteomic signature of a substantially homogeneous population of antigen presenting cells that have been contacted with individual toxins, pathogens or cancers; an expert system having a computer readable instruction set for parsing the reference data records and excluding and including individual data records based on user input defined criteria, and a pattern recognition routine to match reference data records with user input data records the pattern recognition routine capable of comparing the proteomic signatures of a reference data record to the signature of the user input data record based on similarity of the proteomic signatures in the records.

18. The system of claim 17, wherein the antigen presenting cells are selected from the group consisting of: cells of lymphoid lineage such as T cells, B cells, lymphoid related dendritic cells and natural killer cells, and cells of the myeloid lineage such as myeloid related dendritic cells, macrophages, monocytes, megakaryocytes, platelets, granulocytes and neutrophils.

19. The system of claim 17, wherein the pathogen or toxin is selected from the group consisting of: Anthrax toxin; Arenavirus; Bacillus anthracis (anthrax); Clostridium botulinum toxin); Brucella species; Burkholderia mallei; Burkholderia pseudomallei (melioidosis); Chlamydia psittaci; Cholera toxin; Clostridium botulinum toxin (botulism); Clostridium perfringens; Ebola virus hemorrhagic fever; Nipah virus; hantavirus; Epsilon toxin of Clostridium perfringens; Escherichia coli including strain O157:H7; Shigella; Francisella tularensis; Glanders (Burkholderia mallei); Lassa fever; Marburg virus hemorrhagic fever; Melioidosis (Burkholderia pseudomallei); Psittacosis (Chlamydia psittaci); Q fever (Coxiella burnetii); Ricin toxin from Ricinus communis (castor beans); Rickettsia prowazekii; Salmonella Typhi and other Salmonella species; Shigella; Smallpox; Staphylococcal enterotoxin B; Typhus fever; Variola major; Vibrio cholerae (cholera); Viral encephalitis; alphaviruses such as Venezuelan equine encephalitis, eastern equine encephalitis, and western equine encephalitis; Filoviruses; Arenaviruses such as Lassa, and Machupo; Vibrio cholerae; Cryptosporidium parvum; and Yersinia pestis.

20. The system of claim 17, wherein the pathogen is a prion.

21. The system of claim 17, wherein the cancer is selected from the group consisting of: Acute Lymphoblastic Leukemia, Adult; Acute Lymphoblastic Leukemia, Childhood; Acute Myeloid Leukemia, Adult; Acute Myeloid Leukemia, Childhood; Adrenocortical Carcinoma; Adrenocortical Carcinoma, Childhood; AIDS-Related Cancers; AIDS-Related Lymphoma; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Non-Hodgkin's During Pregnancy; Lymphoma, Primary Central Nervous System; Macroglobulinemia, Waldenström's; Male Breast Cancer; Malignant Mesothelioma, Adult; Malignant Mesothelioma, Childhood; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelodysplastic/Myeloproliferative Diseases; Myelogenous Leukemia, Chronic; Myeloid Leukemia, Adult Acute; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma and Thymic Carcinoma Thyroid Cancer; Thyroid Cancer, Childhood; Transitional Cell Cancer of the Renal Pelvis and Ureter; Trophoblastic Tumor, Gestational; Unknown Primary Site, Carcinoma of, Adult; Unknown Primary Site, Cancer of, Childhood; Unusual Cancers of Childhood; Ureter and Renal Pelvis, Transitional Cell Cancer; Urethral Cancer; Uterine Cancer, Endometrial; Uterine Sarcoma; Vaginal Cancer; Visual Pathway and Hypothalamic Glioma, Childhood; Vulvar Cancer; Waldenström's Macroglobulinemia; and Wilms' Tumor.
